(19) United States
(12) Patent Application Publication
(10) Pub. No.: US 2024/0124936 A1
(43) Pub. Date: Apr. 18, 2024

(54) PROTEIN AND METABOLITE BLOOD BIOMARKERS FOR THE DIAGNOSIS OF BRUGADA SYNDROME

(71) Applicant: Cardiomix S.r.l., Milano (IT) (IT)

(72) Inventors: Carlo PAPPONE, Milano (IT) (IT); Luigi ANASTASIA, Milano (IT) (IT); Giuseppe CICONTE, Milano (IT) (IT); Vladimir ESPINOSA ANGARICA, Milano (IT) (IT); Gabriele VICEDOMINI, Milano (IT) (IT); Enrico PETRETTO, Milano (IT) (IT)

(21) Appl. No.: 18/278,112

(22) PCT Filed: Feb. 24, 2022
(86) PCT No.: PCT/IB2022/051626

§ 371 Date: Aug. 21, 2023

(30) Foreign Application Priority Data
Feb. 26, 2021 (EP) 21159669.7

(51) Int. Cl. C12Q 1/6883; C12Q 1/6869; G01N 33/573; G01N 33/68

(52) U.S. Cl. CPC C12Q 1/6883; C12Q 1/6869; G01N 33/573; G01N 33/6893; C12Q 2600/156; G01N 2333/902; G01N 2800/32

(57) ABSTRACT

The present invention relates to a specific set of circulating protein and metabolite biomarkers for the diagnosis of Brugada Syndrome (BrS) in a human being and relative methods of detection. The invention further relates to mutated Prg4, Epx and Pon1 genes and related proteins for the prediction and diagnosis of Brugada Syndrome.

The present invention relates to a specific set of circulating protein and metabolite biomarkers for the diagnosis of Brugada Syndrome (BrS) in a human being and relative methods of detection. The invention further relates to mutated Prg4, Epx and Pon1 genes and related proteins for the prediction and diagnosis of Brugada Syndrome.

The Brugada Syndrome (BrS) is an inherited arrhythmogenic disease defined by a coved-type ST-segment elevation in the right precordial leads on the electrocardiogram and increased risk of sudden cardiac death (SCD) in patients with structurally normal hearts [1-4]. Brugada Syndrome has been reported to be responsible for 5-40% of sudden cardiac deaths and is an important cause of death in individuals aged<40 years [5-7], even if it occurs also in infants and children [1]. The syndrome is endemic in Asiatic regions [6] and appears to be 8 to 10 times more common in men than women.

The syndrome typically manifests with cardiac arrest or syncope, occurring in the third and fourth decade of life [3, 4, 6], however, the majority of patients are asymptomatic with structurally normal heart, and they are usually diagnosed incidentally.

The risk stratification of Brugada Syndrome, particularly in asymptomatic patients, still represents a clinical challenge.

It was demonstrated that the extent of the arrhythmic substrate is the only independent predictor of inducibility of ventricular fibrillation (VF) and ventricular tachycardia (VT) and might be considered as a new marker for risk stratification and therapy [8].

Currently, subjects with spontaneous type 1 ECG pattern and aborted sudden cardiac death or syncope of arrhythmic origin are at highest risk for future arrhythmic events and are advised to receive an implantable cardioverter defibrillator (ICD), despite the risk of device-related complications and inappropriate shocks.

To date Brugada Syndrome diagnosis is based on the identification of the typical ECG-trace signal that may be spontaneous (type 1) or can be induced by a pharmacological challenge test with intravenous administration of sodium channel blockers, such as ajmaline or flecainide. To be considered as diagnostic for BrS, the pattern seen on the ECG includes coved-type ST elevation in the right precordial leads, mainly V1 and V2 placed in the high right intercostal spaces (from the second to the fourth).

The ajmaline challenge is the most accurate test to-date to diagnose Brugada Syndrome in individuals without a spontaneous type 1 ECG pattern. In patients with normal cardiac cells, ajmaline has little or no effect on the ECG. The drug has proven to be a powerful tool in unmasking the Brugada type 1 pattern [10] and to be more accurate than flecainide, which is associated with a 36% false negative rate [11]. However, ajmaline administration can provoke VT/VF during the diagnostic procedure. As a result, the Brugada diagnostic test with ajmaline challenge may be also not allowed in several countries as screening test for safety reasons. Moreover, given the risk associated with the procedure, patients themselves refuse to undergo the procedure. Additionally, the procedure requires an experienced staff, in hospitals with an Extra Corporeal Membrane Oxygenation (ECMO) team. Therefore, this procedure is not widely available, implying that patients may need to travel long distances for the procedure. From an economical perspective, the procedure is expensive and a considerable burden for Public National Healthcare System. As a result, potential patients do not undergo the diagnostic test in a timely fashion, or at all, exposing them to considerable risk of sudden cardiac death.

Moreover, Brugada Syndrome has a further not negligible genetic treat: it is a heterogeneous channelopathy, inherited as an autosomal dominant trait with incomplete penetrance [9].

To date, about three hundred pathogenic variants in 25 genes have been identified in subjects with Brugada Syndrome, but most of them (25-30%) involve the SCN5A gene on chromosome 3p22, encoding the α-subunit of the cardiac sodium channel NaV1.5 [10]. Other genetic lesions associated with Brugada Syndrome include heterozygous variants in CACNA1C, SCN10A, PKP2, TRPM4, KCNH2, and at least eighteen other genes. SCN5A heterozygous mutations account for 11-28% of all Brugada Syndrome cases [11].

However, about 90% of patients with Brugada Syndrome do not have a mutation in the SCN5A gene.

To further complicate the genetic assessment, there are different related Brugada Syndromes caused by mutations in genes other than the SCN5A gene. For example, Brugada Syndrome-2 (Phenotype MIM #611777) is caused by mutation in the GPD1L gene. Brugada Syndrome-3 (Phenotype MIM #611875) and Brugada Syndrome-4 (Phenotype MIM #611876), the phenotypes of which include a shortened QT interval on ECG, are caused by mutation in the CACNA1C and CACNB2 genes, respectively. Brugada Syndrome-5 (Phenotype MIM #612838) is caused by mutation in the SCN1B gene. Brugada Syndrome-6 (Phenotype MIM #613119) is caused by mutation in the KCNE3 gene. Brugada Syndrome-7 (Phenotypte MIM #613120) is caused by mutation in the SCN3B gene. Brugada syndrome-8 (Phenotype MIM #613123) is caused by mutation in the HCN4 gene. While the scientific community still regards Brugada Syndrome as an ion channelopathy, further evidence suggests that the syndrome may also originate by additional factors. For example, an alpha-tropomyosin heterozygous mutation appears to link HCM and Brugada Syndrome in one study, suggesting a role for sarcomeropathies [12]. Another study, published by the present inventors, demonstrated for the first time a pathogenic variant in the MYBPC3 gene and its potential association with Brugada Syndrome [13]. In fact, altered sarcomeric properties have been directly implicated in arrhythmogenesis and sudden death [14-16].

In the light of the complexity of this multi-factorial disorder and the existence of many genetic variants of the disease at the molecular level, genetic testing may be used just to confirm a clinical diagnosis of Brugada Syndrome but is not suitable to make diagnosis of the disease in any subject.

These limitations of genetic screening highlight the urgent need to seek biomarkers aimed both at confirmation of the Brugada Syndrome to establish a prognosis in already affected patients.

In this direction, the systematic introduction of active screening in unmasking Brugada Syndrome using sodium-channel blockers, has considerably increased the diagnosis and incidence of the disease, although its true prevalence is still unknown [17]. Nonetheless, the drug challenge used for the diagnosis can be associated with the occurrence of potentially life-threatening ventricular arrhythmias, thus limiting its widespread use in the absence of a safe hospital environment.

In view of the foregoing, there is a need for a more time and cost-efficient, sensitive and non-invasive laboratory test for diagnosing Brugada Syndrome, which could eventually replace the drug challenge, or at least limit it to those individuals that are positive to such initial laboratory screening and overcome the limits of genetic screening.

The authors of the present invention have now identified a set of circulating biomarkers that allows to overcome the different technical problems affecting the prior art, providing a reliable and sensitive method for the diagnosis of Brugada Syndrome in any patients, herein included also asymptomatic subjects, patients without previous family history of Brugada Syndrome and patients without previous family history of sudden cardiac death.

Specifically, the authors found that patients affected by Brugada Syndrome possess signs of the disease that are detectable in the peripheral blood. In particular, based on the increasing number and diversity of the mutated genes found in patients affected by Brugada Syndrome, the authors hypothesized that the disease could affect cellular and sub-cellular compartments (such as mitochondria) other than those present in the heart. To this end, considering the complexity of the pathology and the heterogeneity among the patient population, they applied a multi-omics (genomic, proteomics, metabolomics and lipidomics) characterization of peripheral plasma and peripheral blood mononuclear cells (PBMCs) on a large scale, supported by an extensive and integrated bioinformatics and Machine Learning (ML) approaches, leading to the identification of a univocal set of biomarkers, statistically significant related to Brugada Syndrome.

A significant advantage of the use of the set of biomarkers of the invention includes the diagnostic and prognostic utility in a wide set of subtypes of Brugada Syndrome, regardless of the genotype or expression of other known genetic biomarker, such as the SCN5A gene.

The set of biomarkers of the invention may also be very useful for risk certification of the patients with Brugada syndrome. Furthermore, a positive result with the set of biomarkers of the invention allows to identify patients affected by Brugada disease, independently from being symptomatic or not and independently of previous family history of Brugada syndrome or previous family history of sudden cardiac death (SCD).

Therefore, it is an object of the present invention a set of 10 biomarkers (core-set) comprising at least:
i) the subset of 7 proteins listed in the following Table 1:

TABLE 1

| Protein | UniProtKB ID |
|---|---|
| Fibronectin 1 (FN1) | P02751 |
| Inter-alpha-trypsin inhibitor heavy | Q06033 |
| chain 3 (ITIH3) | |
| Proteoglycan 4 (PRG4) | Q92954 |
| Adaptor related protein complex 2 | M0QYZ2 |
| subunit sigma 1 (AP2S1) | |
| Component of oligomeric Golgi | Q9UP83 |
| complex 5 (COG5) | |
| Eosinophil peroxidase (EPX) | P11678 |
| Ribosomal oxygenase 1 (RIOX1) | Q9H6W3 |

ii) the subset of the following 3 metabolites:
   fumaric acid;
   O-palmitoleoylcarnitine;
   L-Glutamic acid;
for the diagnosis of Brugada syndrome in a human being.

The core-set of 10 biomarkers according to the present invention allows a diagnosis of the disease of Brugada syndrome on PBMC/plasma of any subject with an overall average accuracy of about 74% and mean area under the curve (AUC) of about 0.81.

The subset of the invention may further comprise additional biomarkers belonging to the above listed subsets i)-ii) of protein and metabolites.

Therefore, it is another object of the present invention the set of 10 biomarkers further comprising one or more of the following additional biomarkers:
i) a protein selected from the group listed in the following Table 2:

TABLE 2

| Protein | UniProtKB ID |
|---|---|
| Paraoxonase 1 (PON1) | P27169 |
| Aldehyde dehydrogenase 3 | P43353 |
| family member B1 | |
| (ALDH3B1) | |
| Solute carrier family 25 | P05141 |
| member (SLC25A5) | |
| S100 calcium binding protein | P31949 |
| A11 (S100A11) | |
| Phospholipase C beta 2 | Q00722 |
| PLCB2) | |

and the metabolite 2-hydroxydecanoate;
or a combination thereof.

Preferably, all the additional biomarkers above listed are included in the set of biomarkers according to the invention, albeit also intermediate solutions wherein only some of them are included are contemplated in the scope of the present invention.

Therefore, according to a further preferred embodiment it is another object of the present invention a set of 16 biomarkers (extended-set) comprising: i) the subset of 12 proteins listed in the following Table 3:

TABLE 3

| Protein | UniProtKB ID |
|---|---|
| Fibronectin 1 (FN1) | P02751 |
| Inter-alpha-trypsin inhibitor | Q06033 |
| heavy chain 3 (ITIH3) | |
| Proteoglycan 4 (PRG4) | Q92954 |
| Adaptor related protein complex 2 | M0QYZ2 |
| subunit sigma 1 (AP2S1) | |
| Component of oligomeric Golgi | Q9UP83 |
| complex 5 (COG5) | |
| Eosinophil peroxidase (EPX) | P11678 |
| Ribosomal oxygenase 1 (RIOX1) | Q9H6W3 |
| Paraoxonase 1 (PON1) | P27169 |
| Aldehyde dehydrogenase 3 family | P43353 |
| member B1 (ALDH3B1) | |
| Solute carrier family 25 member | P05141 |
| (SLC25A5) | |
| S100 calcium binding protein A11 | P31949 |
| (S100A11) | |
| Phospholipase C beta 2 (PLCB2) | Q00722 |

and
ii) the subset of the following 4 metabolites:
   fumaric acid;
   O-palmitoleoylcarnitine;
   L-Glutamic acid;
   2-hydroxydecanoate;
for the diagnosis of Brugada Syndrome in a human being.

Using the 16 biomarkers (extended-set) on PBMC/plasma cell populations in different patient subgroups, the overall accuracy of the test is 75%-80% and the AUC is 0.83-0.89, as reported in Table 4 of Example 1.

According to a preferred embodiment of the invention said biological sample is selected from the group consisting of whole blood, plasma, serum, peripheral blood and PBMCs. Preferably, the biological sample is plasma and/or PBMCs.

According to alternative embodiments, said biological sample can come indifferently from adult subjects or children, male or female subjects.

In a preferred embodiment of the invention said human being may be either asymptomatic (that is with structurally normal heart) or at high risk for Brugada Syndrome due to family history, previous events of heart atrial and/or ventricular fibrillation, diabetes or obesity.

It is another object of the present invention an in vitro method for detecting the presence or measuring concentration of the occurrence of one of the set of biomarkers above listed in a biological sample of a human being.

In order to provide a reliable result, the in vitro method should at least foresee the detection of the presence or measuring concentration of at least the 10 core-set of biomarkers up to the 16 extended set of biomarkers.

In a particular embodiment the invention provides an in vitro method for detecting the presence of one of the set of biomarkers above listed in a biological sample of a human being, comprising the following steps:
a) detection of at least 7 up to 12 proteins of subset i) by an assay selected from the group comprising Western Blot, Dot blot, ELISA, flow cytometry, enzymatic activity assay, targeted MS, multiplex protein analysis, ProQuantum high-sensitivity immunoassays, or a combination thereof;
b) detection of at least 3 up to 4 of the metabolites of subset ii) by an assay such as one selected from the group comprising MS, GC-MS, fluorimetric or colorimetric assays, NMR, spectroscopy with nanoprobes, Enzyme-Linked oligonucleotide assays, or a combination thereof;
wherein the positive detection of all the biomarkers compared to a normal control allows the identification of patients affected by Brugada Syndrome.

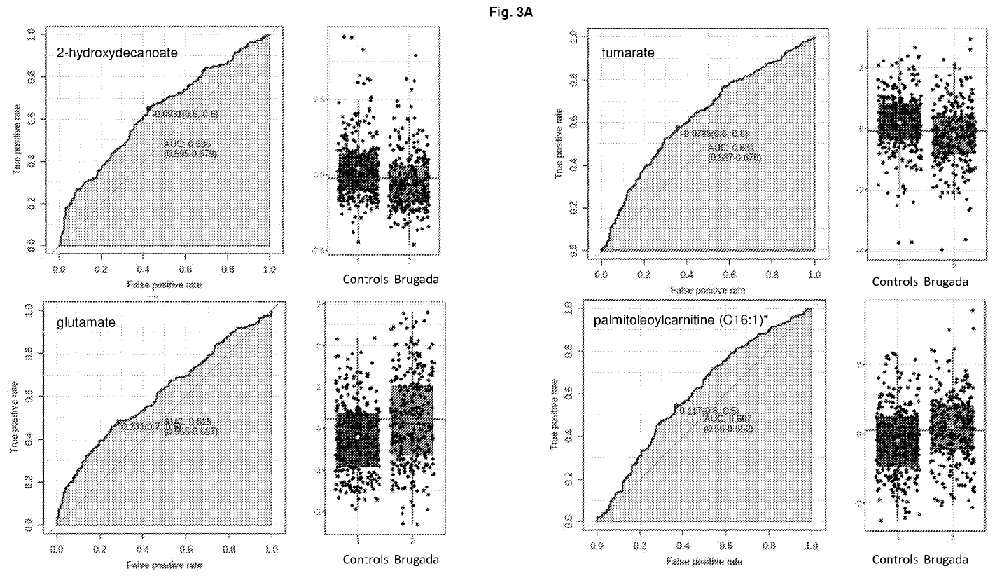

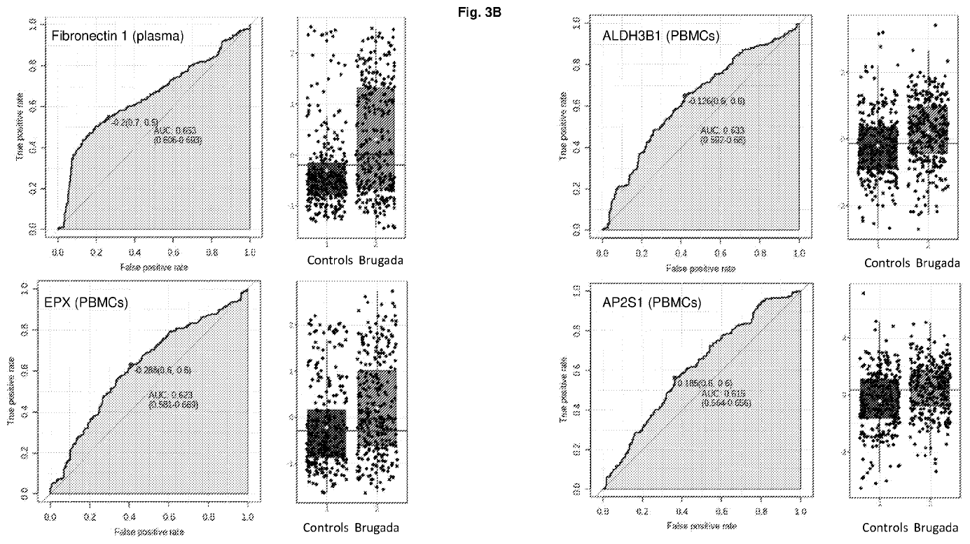

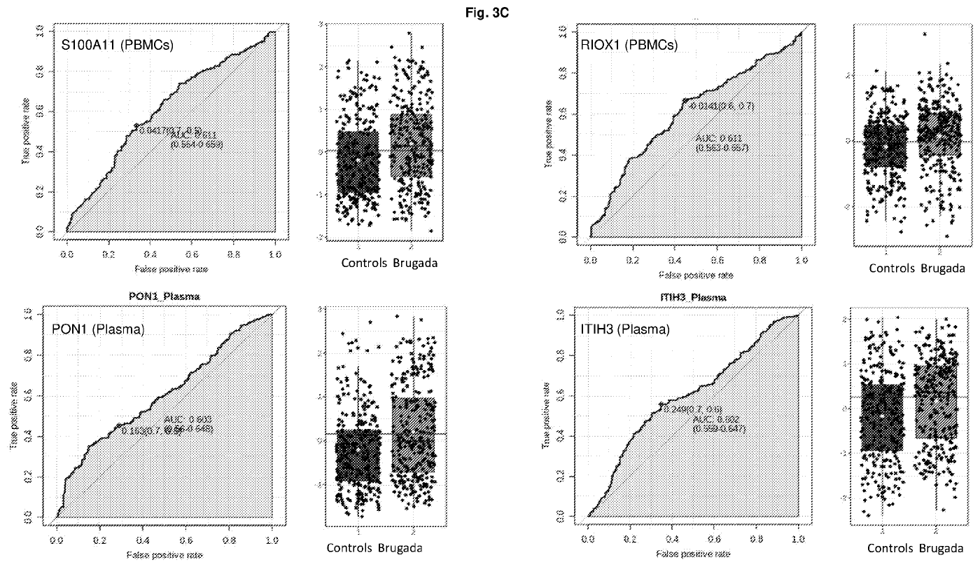

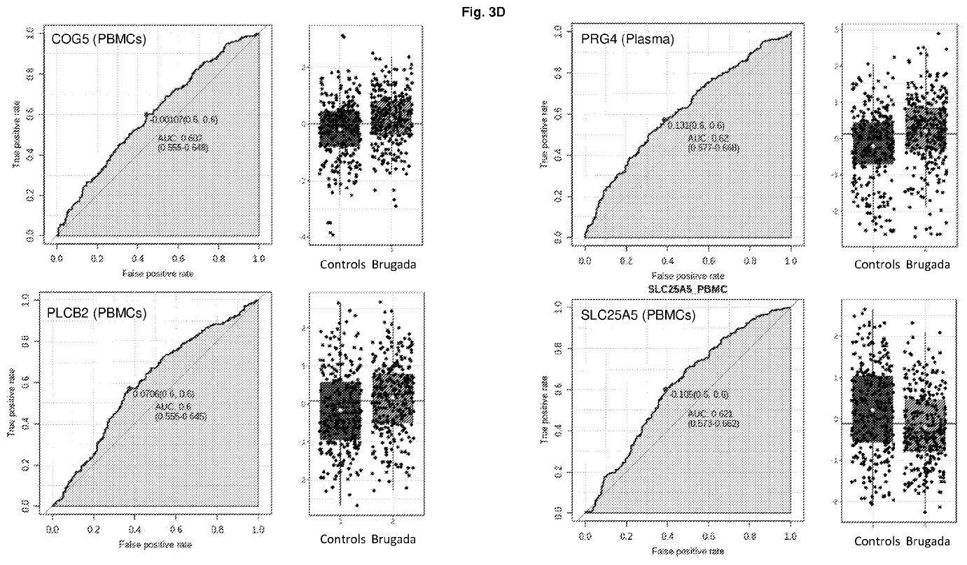

According to an alternative embodiment the invention relates to an in vitro method for measuring the concentration of one of the above listed set of biomarkers in a biological sample of a human being, comprising the following steps:
a) quantitative determination of at least 7 up to 12 proteins of subset i) by a quantitative technique such as Western Blot, ELISA, enzymatic activity assay, targeted MS, multiplex protein analysis, ProQuantum high-sensitivity immunoassays, or a combination thereof;
b) quantitative determination of at least 3 up to 4 of the metabolites of subset
ii) by a technique such as HPLC, MS, NMR analysis, ELONA, or a combination thereof;
wherein an increase or decrease of the concentration of the biomarkers compared to a normal control indicates a diagnosis of Brugada Syndrome (as in FIG. 3).

A combination of all biomarkers means that can predict the patient is affected by Brugada Syndrome, independently on the fact that such patient is asymptomatic or symptomatic.

According to a preferred embodiment of the invention said biological sample is selected from the group consisting of plasma, PBMCs serum, peripheral blood and whole blood, or a combination thereof. Preferably, the biological sample is plasma and/or PBMCs. According to alternative embodiments of the afore mentioned method, the starting biological sample can come indifferently from adult subjects or children, male or female subjects.

In a preferred embodiment of the invention said human being may be either asymptomatic (that is with structurally normal heart) or at high risk for Brugada Syndrome due to family history, previous events of heart atrial and/or ventricular fibrillation, diabetes or obesity.

More preferably said human being is about 40 years old or younger.

The invention is further directed to a kit for the detection of the subset i) of at least 7 proteins of Table 1 up to the subset i) of 12 proteins of Table 3, said kit comprising antibodies specific for anyone of the protein of the subset i), wherein said antibodies are labelled or attached to a solid support.

Preferably, said antibodies are labelled with fluorescents.

Said solid support is preferably a multi-well plate. Preferably, said kit is an ELISA kit. In a preferred embodiment, cell detection after incubation with antibodies of the invention is carried out by flow cytometry.

The present invention further relates to an oligonucleotide sequence of Prg4 gene (Gene Ensemble ID ENSG00000116690; SEQ ID NO:1) characterized by the presence of a deleterious mutations C→T in position 186,304,862 of exon 6 the nucleotide sequence, said oligonucleotide sequence being RNA or DNA, for use as a genetic diagnostic marker for the diagnosis of Brugada Syndrome in a human being. Preferably said mutated oligonucleotide sequence comprises or consists of SEQ ID NO: 7.

It is another object of the present invention a mutated PRG4 protein encoded by the above mentioned mutated oligonucleotide sequence of Prg4 gene, for use as a genetic diagnostic marker for the diagnosis of Brugada Syndrome in a human being. In a particular preferred embodiment, said mutated PRG4 protein comprises or consists of SEQ ID NO:2, wherein the amino acid change R→W in position 180 occurs.

The present invention relates to an oligonucleotide sequence of Epx human gene (Gene Ensemble ID ENSG00000121053; SEQ ID NO:3) characterized by the presence of a deleterious mutations G→C in position 58,193,733 of exon 4 of the gene sequence, said oligonucleotide sequence being RNA or DNA, for use as a genetic diagnostic marker for the diagnosis of Brugada Syndrome in a human being. Preferably said mutated oligonucleotide sequence comprises or consists of SEQ ID NO:8.

It is another object of the present invention a mutated EPX protein encoded by the above mentioned mutated oligonucleotide sequence of Epx human gene, for use as a genetic diagnostic marker for the diagnosis of Brugada Syndrome in a human being. In a particular preferred embodiment, said mutated EPX protein comprises or consists of SEQ ID NO:4, wherein the mutation Q→H occurs in position 122. Another object of the present invention relates to an oligonucleotide sequence of Pon1 human gene (ENSG00000005421; SEQ ID NO:5) characterized by the presence of a deleterious mutations A→T in position 95,316,772 of exon 3 of the gene sequence, said oligonucleotide sequence being RNA or DNA, for use as a genetic diagnostic marker for the diagnosis of Brugada Syndrome in a human being. Preferably said mutated oligonucleotide sequence comprises or consists of SEQ ID NO: 9.

It is a further object of the present invention a mutated PON1 protein encoded by the above mentioned mutated oligonucleotide sequence of Pon1 human gene, for use as a genetic diagnostic marker for the diagnosis of Brugada Syndrome in a human being. In a particular preferred embodiment, said mutated PON1 protein comprises or consists of SEQ ID NO:6, wherein the amino acid change L→M in position 55 occurs.

The invention further relates to the use of one or more of the mutated oligonucleotide sequences or mutated proteins as above described, for the diagnosis of Brugada Syndrome in a human being by detection in a biological sample, wherein said biological sample is selected from the group consisting of plasma, PBMCs, whole blood, serum and peripheral blood, or a combination thereof.

Furthermore, the invention provides an in vitro method for detecting the presence of one or more of the oligonucleotide sequences of Prg4, Epx and Pon1 genes above detailed by genetic analysis through PCR or DNA sequencing.

Finally, one further object of the invention is a kit comprising primer or probes complementary to one or more of the oligonucleotide sequences of Prg4, Epx and Pon1 genes above detailed for the diagnosis of Brugada Syndrome in a human being.

Therefore, given the role of several biomarker proteins dysregulated in Brugada Syndrome patients in metabolic pathways and disease (e.g., PRG4, PON1), alcohol metabolism (e.g., ALDH3B1) and the direct identification of dysregulated metabolites of the mitochondrial TCA cycle (e.g., fumarate), a causal link between these metabolic processes dysregulated and Brugada Syndrome is the central discovery of the present invention. The further identification of several biomarker proteins to be mutated in Brugada Syndrome patients further suggests a causal link with the disease.

The present invention will now be described, for non-limiting illustrative purposes, according to a preferred embodiment thereof, with particular reference to the attached figures, wherein:

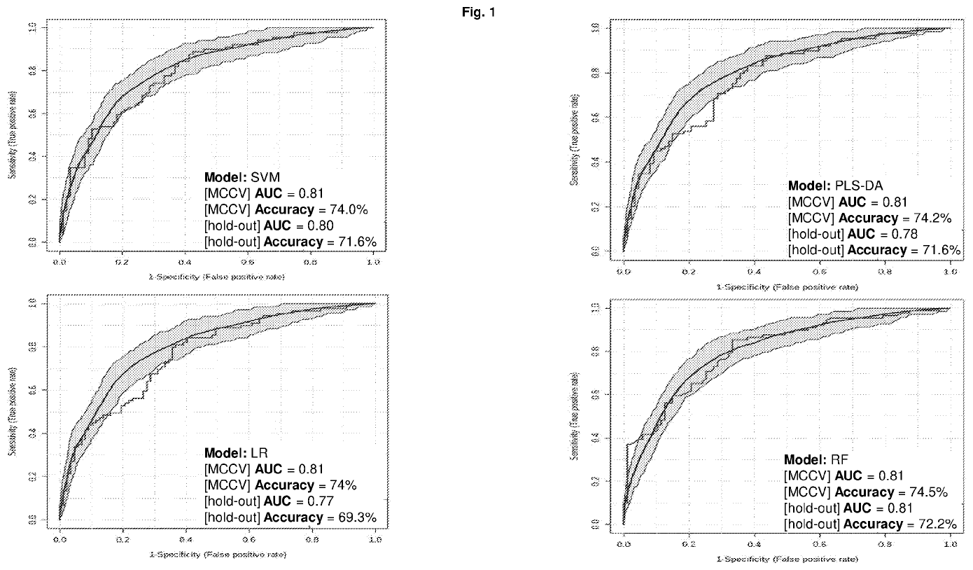

FIG. 1 shows accuracy and AUC estimates of the core-set of 10 biomarkers of the invention by 4 independent models (SVM, PLS-DA, RF and LR). For each model, Monte Carlo cross-validation (MCCV) with 100 runs was applied, using, in each run, different random sets of 67% (⅔) of patients for training and 33% (⅓) for testing. The AUC curve obtained by the MCCV method is indicated by blue colour, where the 95% confidence interval (95% CI) of the AUC is estimated by 100 MCCV samples. In addition, the AUC curves and AUC estimates using the hold-out method were reported, where 409 subjects (70%) were used as training-dataset and 176 subjects (30%) were used as test-dataset. The AUC curve obtained by the hold-out method is indicated by magenta colour.

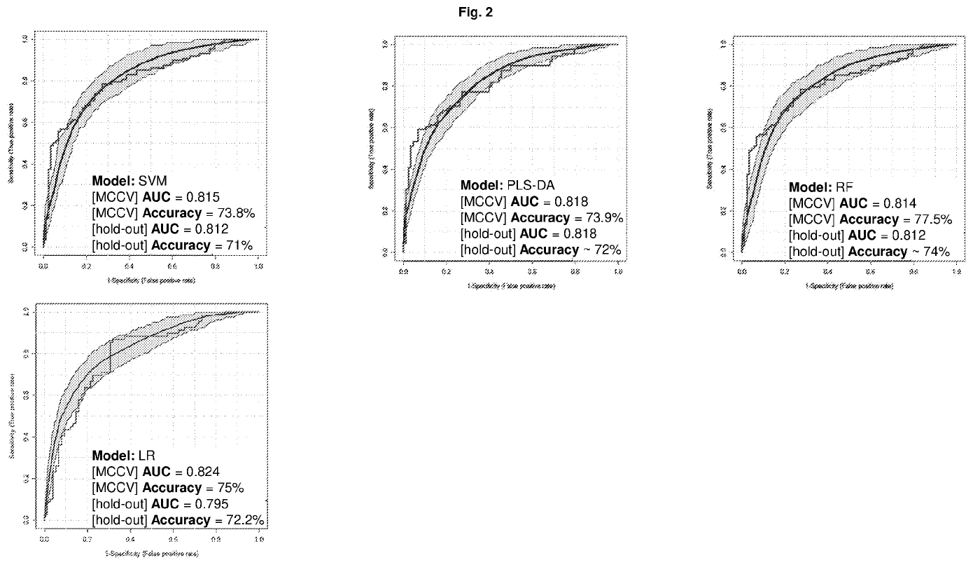

FIG. 2 shows the accuracy and AUC estimates of the extended-set of biomarkers of the invention by 4 independent models (SVM, PLS-DA, RF and LR). For each model, Monte Carlo cross-validation (MCCV) with 100 runs was applied, using, in each run, different random sets of 67% (⅔) of patients for training and 33% (⅓) for testing. The AUC curve obtained by the MCCV method is indicated by blue colour, where the 95% confidence interval (95% CI) of the AUC is estimated by 100 MCCV samples. In addition, the AUC curves and AUC estimates using the hold-out method were reported, where 465 subjects (79%) were used as training-dataset and 120 subjects (21%) were used as test-dataset. The AUC curve obtained by the hold-out method is indicated by magenta colour.

FIGS. 3A-D show the AUC and box plots in PBMCs and plasma for each metabolites (3A) and protein (3B-D) belonging to the biomarker core-set and extended-set for the diagnosis of Brugada Syndrome.

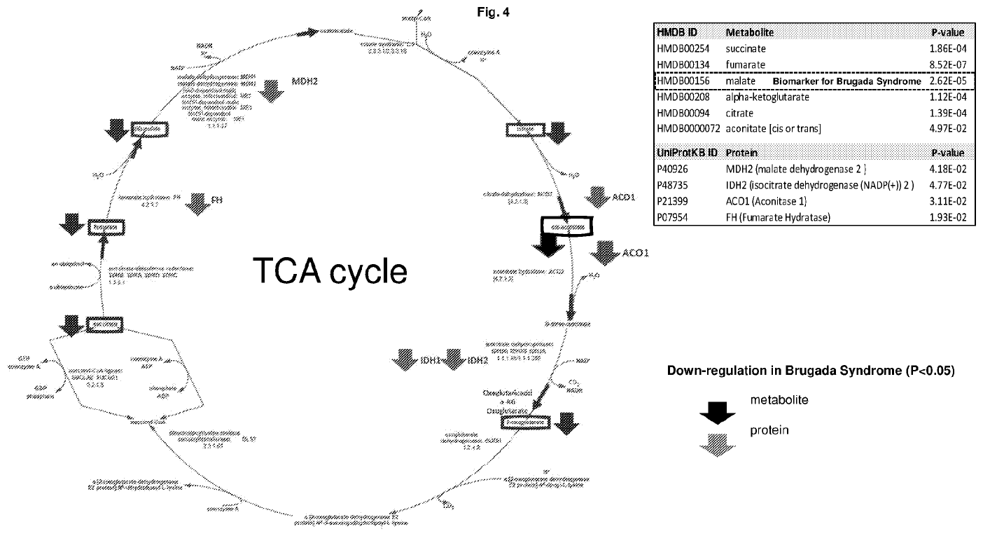

FIG. 4 shows the dysregulation of protein and metabolites of the TCA cycle in Brugada Syndrome.

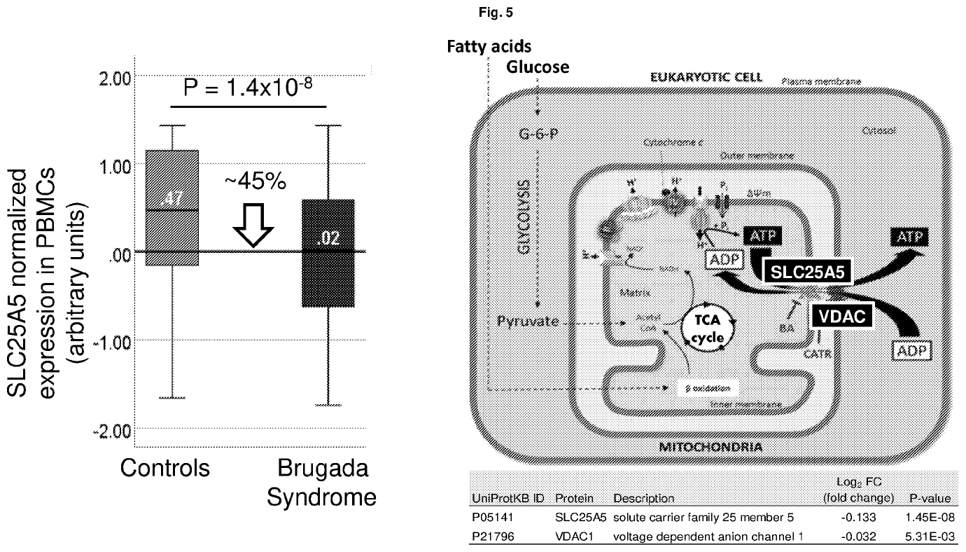

FIG. 5 shows dysregulation of SLC25A5 and VDAC1 mitochondrial proteins in Brugada Syndrome. Both SLC25A5 and VDAC1 proteins are essential for ATP/ADP transport activity and energy metabolism by mitochondria.

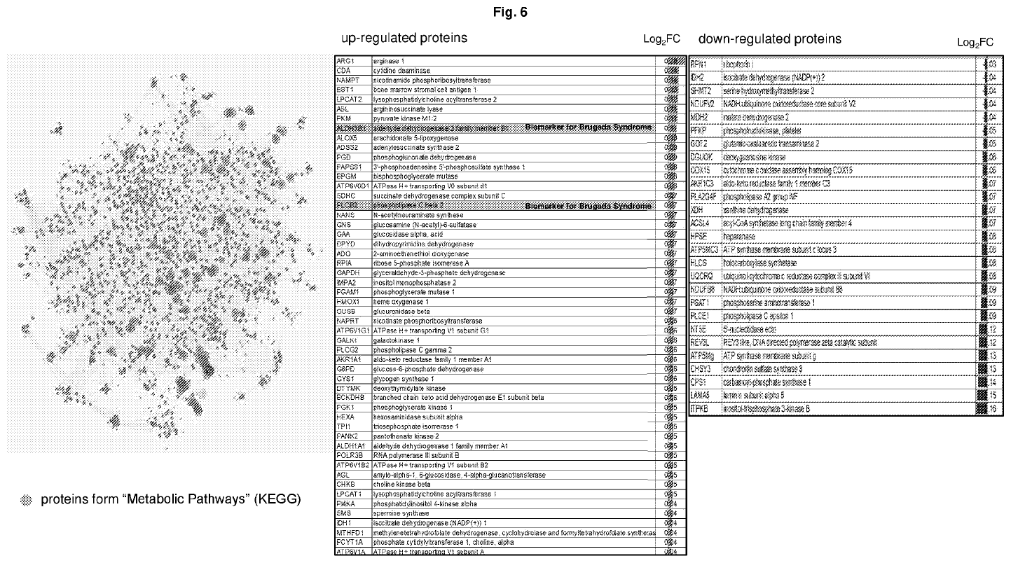

FIG. 6 shows protein-protein interaction (PPI) network derived from STRING v11.0b (https://string-db.org/) of the 995 dysregulated proteins in blood PBMCs from Brugada Syndrome patients (FDR≤5%), showing the 79 proteins significantly enriched in “Metabolic Pathways” (KEGG) (FDR=0.00099).

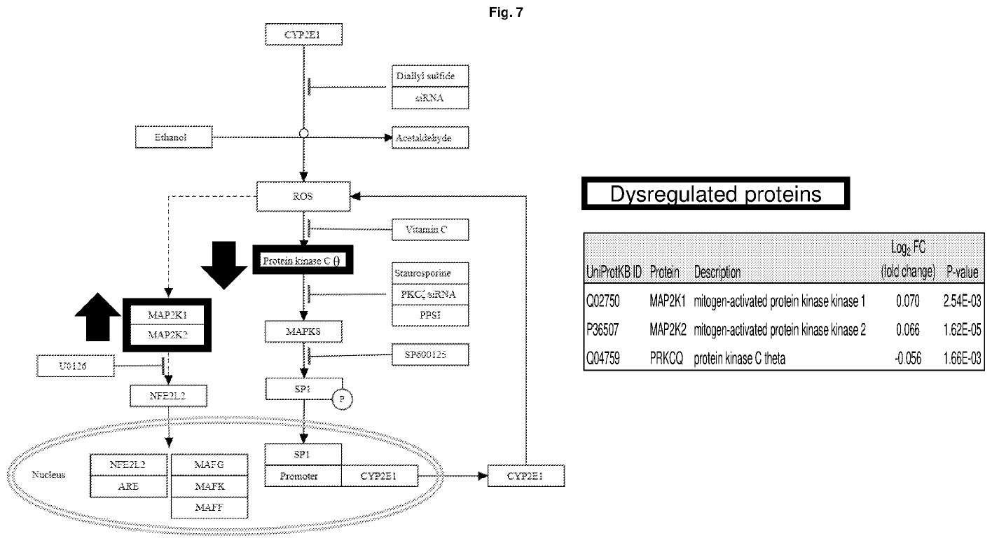

FIG. 7 shows the dysregulated proteins in patients with Brugada Syndrome that are part of the ethanol metabolism pathway resulting in production of ROS by CYP2E1. plateau reached by the curve of the accuracy of the test when more than 60 features have been employed.

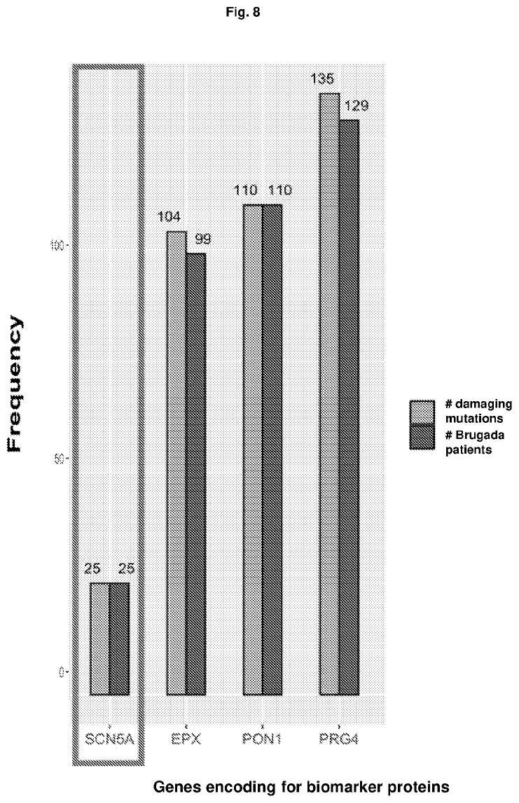

FIG. 8 shows a graph illustrating the number of deleterious mutations at the EPC, PON1 and PRG4 genes encoding for the biomarker proteins (extended set, listed in TABLE 2) and number of Brugada Syndrome patients (out or 186 tested) carrying at least one damaging deleterious mutation in each gene. For comparative purposes, the number of damaging deleterious mutations at SCN5A (i.e., the gene in which mutations are most commonly found in Brugada Syndrome) that have been detected in the 186 Brugada Syndrome patients tested by WGS analysis was reported.

The following examples are merely illustrative and should not be considered limiting the scope of the present invention.

### Example 1: Accuracy of the Test

Accuracy and AUC have been independently estimated for the core-set and the extended-set of biomarkers using 4 ML methods: Support Vector Machine (SVM), partial least squares discriminant analysis (PLS-DA), random forest (RF) and logistic regression (LR), which provided very similar and highly consistent estimates (FIG. 1).

To assess accuracy, the Monte Carlo cross-validation (MCCV) method with 100 runs was applied, using, in each run, different random sets of 67% (⅔) of patients for training and 33% (⅓) for testing.

The core-set of 10 biomarkers according to the present invention allows a diagnosis of the disease of Brugada syndrome on PBMC/plasma of any subject with an overall average accuracy of about 74% and mean area under the curve (AUC) of about 0.81.

By increasing the number of biomarkers from 10 (core-set) to 16 (extended-set) it is possible to reach an overall accuracy of the test on PBMC/plasma population of about 74%-77% and AUC to 0.814-0.822 across methods (FIG. 2).

Using the 16 biomarkers (extended-set) on PBMC/plasma cell populations in different patient subgroups, the overall accuracy of the test is 75%-80% and the AUG is 0.83-0.89, as reported in Table 4.

TABLE 4

| | Number of | AUC¹ | |
|---|---|---|---|
| Patients subgroups | patients | (P-value)² | Accuracy¹ |
| All samples | 585 | 0.842 (10⁻⁴⁶) | 75.6% |
| No previous family | 231 | 0.895 (10⁻²⁴) | 78.8% |
| history of Brugada | | | |
| Syndrome | | | |
| No previous family | 366 | 0.883 (10⁻³⁷) | 79.5% |
| history of SCD | | | |
| Asymptomatic patients | 366 | 0.863 (10⁻³³) | 76.5% |
| No pathogenic mutations | 499 | 0.837 (10⁻³⁸) | 76.4% |
| at the SCN5A gene | | | |
| “Old” patients | 293 | 0.833 (10⁻²¹) | 75.4% |
| (age >37.7 years)³ | | | |
| “Young” patients | 292 | 0.866 (10⁻²⁶) | 80.8% |
| (age <= 37.7 years)³ | | | |

¹AUC and accuracy are calculated by Logistic Regression model
²P-value of significance, where the null hypothesis is true area = 0.5
³“Old” is defined as age > median age in the patient cohort; “young” is defined as age <= median age in the patient cohort

Therefore, the set of 16 biomarkers (extended-set) have an increased accuracy and a significantly high AUC for the diagnosis of Brugada Syndrome in a human being with no previous family history of Brugada Syndrome (AUC˜0.89, accuracy ˜79%) or in a human being with no previous family history of SCD (AUC˜0.88, accuracy ˜79%) or in a human being with no symptoms of Brugada Syndrome (accuracy ˜76%).

The set of 16 biomarkers (extended-set) have a high accuracy (˜76%) and significant AUC (˜0.84) in patients with Brugada Syndrome who do not have a mutation in the SCN5A gene. The set of 16 biomarkers (extended-set) have a high accuracy for the diagnosis of Brugada Syndrome in both “young” patients (AUC˜0.87, accuracy ˜81%) and “old” patients (AUC˜0.83, accuracy ˜75%). A positive result related to the overall combination of biomarkers belonging to the set (being either the core set or the extended set or an intermediate set) means that the patient is affected by Brugada Syndrome, independently on the fact that such patient is asymptomatic or symptomatic. In other words, rather than a single upregulated (or downregulated) protein or metabolite, a combination of upregulated or downregulated proteins or metabolites predict the patient is affected by Brugada Syndrome, independently on the fact that such patient is asymptomatic or symptomatic.

Each biomarker metabolite or protein that defines the composite set of 16 biomarkers (extended-set) is independently predictive of the diagnosis of Brugada Syndrome, with AUC>0.6, as depicted in FIG. 3A-D.

The FN1 (Fibronectin 1) protein is one of the and 10 biomarkers (core-set) and of the and 16 biomarkers (extended-set) for the diagnosis of Brugada Syndrome in a human being. FN1 protein levels have been previously reported to be altered by the presence of an implantable cardioverter defibrillator (ICD) [20]. In our patient cohort (n=585), FN1 protein levels in plasma are significantly elevated (˜3 fold) in Brugada patients with ICD implant (n=91) compared Brugada patients without ICD (n=203), P=0.001 by Two-Sample Kolmogorov-Smirnov test. However, not all Brugada patients with an ICD implant have consistently high expression of FN1. Despite this, a high predictive ability of FN1 as biomarker of the diagnosis of Brugada Syndrome is retained in the subset of patients who do not have an ICD implant (n=494, accuracy ˜66%, AUC=0.614, P=10-5) with an odds ratio (OR)=1.95 of having Brugada Syndrome associated with increased FN1 protein levels in plasma. Table 5 reports the accuracy of FN1 (fibronectin 1) biomarker in patients with and without ICD implant.

TABLE 5

| Patients | | AUC¹ | 95% CI | OR | |
|---|---|---|---|---|---|
| subgroups | N | (P-value)² | AUC¹ | [95% CI]³ | Accuracy¹ |
| All samples | 585 | 0.653 (10⁻¹⁰) | 0.606-0.693 | 1.70 | 64.4% |
| (with and without | | | | [1.39-2.08] | |
| ICD implant) | | | | | |
| Only patients | 494 | 0.613 (10⁻⁰⁵) | 0.561-0.665 | 1.95 | 65.8% |
| without ICD | | | | [1.62-2.35] | |
| implant | | | | | |

¹AUC and accuracy for the biomarker FN1 (Fibronectin 1) are calculated by Logistic Regression model
²P-value of significance, where the null hypothesis is true area = 0.5
³Odds Ratio (OR) and 95% CI of OR for the biomarker FN1 (Fibronectin 1) are calculated by Logistic Regression model

Plasma levels of fumarate (fumaric acid) is one of the and 10 biomarkers (core-set) and of the and 16 biomarkers (extended-set) for the diagnosis of Brugada Syndrome in a human being. Decreased plasma levels of fumarate are significantly associated with Brugada Syndrome (adjusted P=8.2×10⁻⁵), suggesting a dysregulation of the TCA cycle (tricarboxylic acid cycle) in patients with Brugada Syndrome. Other proteins encoding for relevant enzymes (MDH2, malate dehydrogenase 2; IDH2, isocitrate dehydrogenase (NADP(+)) 2; ACO1, Aconitase 1; FH, Fumarate Hydratase) and metabolites (succinate, malate, alpha-ketoglutarate, aconitate, citrate) of the TCA cycle are significantly dysregulated (P<0.05) in blood from Brugada Syndrome patients, see FIG. 4. This indicates an overall downregulation of TCA cycle in mitochondria, linking mitochondrial dysfunction to Brugada Syndrome.

The TCA cycle is a key metabolic pathway that connects carbohydrate, fat, and protein metabolism. Therefore, according to the identification of reduced levels of fumarate (a biomarker of Brugada Syndrome, Table 1, Table 5, above) and of several other metabolites (TCA cycle intermediates) and nuclear and mitochondrial proteins (enzymes of the TCA cycle in mitochondria) in Brugada Syndrome patients, a further preferred embodiment it is another object of the present invention the mitochondrial dysfunction and/or dysregulation of TCA cycle for the diagnosis of Brugada Syndrome in a human being.

Another biomarker protein (extended-set), SLC25A5 (solute carrier family 25 member 5) also known as ANT2, is significantly downregulated (˜45% on average) in PBMCs from Brugada Syndrome patients (P=1.4×10⁻⁸), and it belongs to the mitochondrial carrier family SLC25. SLC25A5 is a key mitochondrial ATP transporter that is expressed ubiquitously at a level depending on the specific respiratory activity of the tissues, including brain, lung, kidney, pancreas, heart, skeletal muscle, spleen/inner and in the mitochondrial membrane. SLC25A5 encodes for a mitochondrial carrier that is mostly localized in the inner mitochondrial membrane (FIG. 5); SLC25 protein family have been implicated in diverse metabolic pathways, including oxidative phosphorylation, TCA cycle, fatty acid oxidation, gluconeogenesis, lipogenesis, ketone body production and utilization, urea synthesis, amino acid degradation, regulation of nucleotide and deoxynucleotide pools in the mitochondrial matrix, heat production [21]. Together with the inner-membrane mitochondrial protein SLC25A5, the outer-membrane mitochondrial protein VDAC1 appears to be significantly downregulated in blood PBMCs from Brugada Syndrome patients; both proteins are key for ATP/ADP transport activity and energy metabolism by mitochondria (FIG. 5). Despite VDAC1 levels change less than those of SLC25A5 in Brugada Syndrome (Log₂FC=−0.133 [SLC25A5] vs −0.033 [VDAC1]), VDAC1 location in the outer mitochondrial membrane, makes it to interact with over 100 proteins, and to orchestrate the interaction of mitochondrial and cellular activities through several signaling pathways [22].

The downregulation of both SLC25A5 and VDAC1 mitochondrial proteins in Brugada Syndrome is another object of the present invention of the mitochondrial dysfunction for the diagnosis of Brugada Syndrome in a human being.

Functional analysis of the proteins that are dysregulated in blood PBMCs from Brugada Syndrome patients (n=995 proteins, false discovery rate (FDR)≤5%), identified significant enrichment for 79 proteins (COX15, NANS, HMOX1, MTHFD1, NAMPT, POLR3B, GAPDH, TPI1, GOT2, LAMA5, PI4KA, NT5E, GNS, LPCAT2, DGUOK, BST1, PAPSS1, HEXA, IMPA2, PGD, ATP6V1A, ATP6V1B2, LPCAT1, RPIA, ATP5G3, ATP6VOD1, PCYT1A, AGL, RPN1, ALDH1A1, NDUFB8, ATP5L, CHSY3, GUSB, DTYMK, GAA, ASL, PANK2, GYS1, BCKDHB, PKM, MDH2, NDUFV2, IDH2, SHMT2, ACSL4, ARGI, REV3L, ADSS, SDHC, DPYD, PGAM1, PLCE1, AKR1A1, PGK1, ADO, ATP6V1G1, ALOX5, CDA, PSAT1, UQCRQ, XDH, AKR1C3, PFKP, BPGM, G6PD, PLA2G4F, HLCS, HPSE, CHKB, SMS, IDH1, NAPRT, CPS1, ITPKB, GALK1, PLCG2, PLCB2, ALDH3B1) involved in metabolism (Metabolic pathways (KEGG, https://www.genome.jp/kegg/), hsa01100), FDR=9.9×10⁻⁴), see FIG. 6. This suggests a dysregulation of metabolic processes in Brugada Syndrome patients, which is in keeping with metabolic abnormalities observed at the clinical level.

Of these proteins important for metabolism, PLCB2 and ALDH3B1 proteins are also part of the biomarker (core-set and extended-set) of Brugada Syndrome (see Tables 2-3 above, FIG. 3).

The PLCB2 (Phospholipase C Beta 2) protein is mainly localized to the plasma membrane and cytosol, and one of the and 16 biomarkers (extended-set) for the diagnosis of Brugada Syndrome in a human being. Compared with controls, PLCB2 protein levels are significantly elevated (Log₂FC=0.073, P=3.3×10⁻⁶) in blood PBMCs of Brugada Syndrome patients. PLCB belongs to the PLC protein family, which are key enzymes that metabolize inositol lipids and have a pivotal role in multiple transmembrane signaling transduction pathways that modulate a series of cellular processes, including cell proliferation and mobility [23]. The PLCB family consists of 4 isozymes, PLCB1 to B4, which share their primary structure and PLCB1 is an important molecule that regulates b cell insulin secretion and can be considered a candidate for therapeutic intervention in diabetes mellitus [24]. However, no data on the role of PLCB2 in diabetes mellitus are available, suggesting the function of PLCB2 in Brugada Syndrome is novel and object of the present invention.

The ALDH3B1 (aldehyde dehydrogenase 3 family member B1) protein is one of the 16 biomarkers (extended-set) for the diagnosis of Brugada Syndrome in a human being. Compared with controls, ALDH3B1 protein levels are significantly elevated (Log₂FC=0.105, P=2.4×10⁻⁸) in blood PBMCs of Brugada Syndrome patients. Aldehyde dehydrogenases (ALDHs) detoxify toxic aldehydes by oxidation to the corresponding carboxylic acids. Long-chain aliphatic aldehydes are largely produced by catabolic metabolism of several lipids, such as ether glycerolipids, fatty alcohols, sphingolipids. Medium-chain aliphatic aldehydes, such as hexanal, octanal and 4-hydroxy-2-nonenal (4HNE) are produced via lipid peroxidation during oxidative stress and ALDH3B1 can oxidise both medium- and long-chain aldehydes. C16 aldehydes such as hexadecanal (HXAL), typically generated through sphingolipid metabolism on the plasma membrane, can be oxidised to palmitic acid (PALM). 4HNE, amongst other reactive medium-chain aldehydes, can be detoxified by ALDH3B1 via oxidation to 4-hydroxynonenoic acid (4HNA), therefore indicating a potential physiological role for ALDH3B1 against oxidative stress. Therefore, ALDH3B1 plays a major role in the detoxification of aldehydes generated by alcohol metabolism and lipid peroxidation [25]. Alcohol intoxication is a potentially under-recognised yet important precipitant of Brugada Syndrome; thus, the identification of ALDH3B1 as a biomarker for the diagnosis of Brugada Syndrome in a human being is further indicative of a link between dysregulated alcohol metabolism and Brugada Syndrome and is another object of the present invention.

Other proteins that are dysregulated in blood PBMCs from Brugada Syndrome patients link alcohol metabolism to Brugada Syndrome. A significant enrichment for the “ethanol metabolism resulting in production of ROS by CYP2E1 (WP4269)” has been identified (see FIG. 7) where increased oxidative stress by CYP2E1 induction is known to be a major consequence of ethanol-mediated liver toxicity [26]. Three proteins dysregulated in Brugada Syndrome that are crucial for the pathways are: MAPK1 (mitogen-activated protein kinase kinase 1), MAPK2 (mitogen-activated protein kinase kinase 2) and PRKCQ (protein kinase C theta), see FIG. 7.

Integrated functional analysis of all proteins (n=995 proteins in PBMCs, FDR≤5%; n=25 proteins in plasma, FDR≤5%) and metabolites (n=275 metabolites in plasma, FDR≤5%) found dysregulated in blood from Brugada Syndrome patients, identified a significant protein-metabolite interaction network (FIG. 8). This network is derived by using existing knowledge of functional interactions between proteins and metabolites from the Human Metabolome Database (HMDB) (which currently covers 114,100 metabolites and 5,779 proteins) [27] and STITCH database (which currently covers 9,643,763 proteins, 430 000 chemicals and 1.6 billion interactions) [28].

The network is significantly enriched for “Alanine, aspartate and glutamate metabolism” pathway (P=3.2×10⁻⁹), comprising 7 metabolites (L-Glutamine, L-Glutamic acid; L-Aspartic acid; Uric Acid, L-Alanine, Choline, Taurolithocholic acid 3-sulfate) and 1 protein (ABCB), which are all dysregulated in in Brugada Syndrome patients.

The central “hub” node the network is GOT2 (glutamic-oxaloacetic transaminase 2) (intramitochondrial isoform)—a member of the malate-aspartate shuttle (MAS)—which plays a key role in the intracellular NAD(H) redox balance. The malate aspartate shuttle requires the concerted action of different components, including mitochondrial malate dehydrogenase MDH2, which is also dysregulated in Brugada Syndrome patients, see FIG. 8. Therefore, both GOT2 and MDH2 proteins identified in the same protein-metabolite interaction network, are essential for malate-aspartate metabolite exchange between mitochondria and cytosol, and for amino acid metabolism. In keeping with the dysregulation of GOT2 and MDH2 proteins in Brugada Syndrome patients, the malate metabolite is also dysregulated (decreased) in Brugada Syndrome patients compared with controls (Log₂FC=−0.12, P=2.6×10⁻⁵), see FIG. 4. Defects in the MAS have been described due to mutations in genes encoding mitochondrial malate dehydrogenase (MDH2) and encoding for glutamic-oxaloacetic transaminase 2 (GOT2) [29]. Thus, the identification of both GOT2 and MDH2 proteins dysregulated in Brugada Syndrome patients is further indicative of a link between dysregulated MAS in Brugada Syndrome and is another object of the present invention.

### Example 2: Multi-Omics Identification of the Set of Biomarkers of Brugada

Syndrome of the Invention

Materials and Methods

Ethical Statement

All procedures performed in studies involving human participants were in accordance with the ethical standards of the institutional and/or national research committee and with the Helsinki declaration and its later amendments or comparable ethical standards. The study design was examined by the Ethical Committee of the San Raffaele Hospital (Protocol BASED-version Jul. 1, 2004).

Patients Enrolment

The population study has been constituted by two groups (300 subjects/group): the Brugada (BrG) and the Control (CG) Groups. The expected enrollment period was of 24 months.

Patients were evaluated for:
baseline laboratory examinations, including total blood count, hepatic, thyroid and coagulation function.
transthoracic ECG.

The inclusion/exclusion criteria for the two groups have been respectively the following.

Brugada Group: All consecutive Brugada Syndrome patients, aged>18 years, positive to the ajmaline test, or considered at high risk of SCD and undergoing epicardial ablation will be enrolled in this study.

Control Group: A population of patients with a structurally normal heart with a negative ajmaline test confirming the absence of Brugada Syndrome.

Inclusion Criteria for Control Group:
Age>18 years
Absence of Brugada Syndrome confirmed with ajmaline test.

Exclusion Criteria for Control Group:
Ejection Fraction<55%
Coronary artery disease with the need for revascularization
Previous heart surgery
Recent surgical/percutaneous intervention for valvular heart disease (<6 months)
Life expectancy<1 year
Impossibility to provide consent to the procedure and to the study

Metabolomic Analysis on Plasma

Samples were prepared using the automated MicroLab STAR® system from Hamilton Company. Several recovery standards were added prior to the first step in the extraction process for QC purposes. To remove protein, dissociate small molecules bound to protein or trapped in the precipitated protein matrix, and to recover chemically diverse metabolites, proteins were precipitated with methanol under vigorous shaking for 2 min (Glen Mills GenoGrinder 2000) followed by centrifugation. The resulting extract was divided into five fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. Samples were placed briefly on a TurboVap® (Zymark) to remove the organic solvent. The sample extracts were stored overnight under nitrogen before preparation for analysis.

QA/QC

Several types of controls were analyzed in concert with the experimental samples: a pooled matrix sample generated by taking a small volume of each experimental sample (or alternatively, use of a pool of well-characterized human plasma) served as a technical replicate throughout the data set; extracted water samples served as process blanks; and a cocktail of QC standards that were carefully chosen not to interfere with the measurement of endogenous compounds were spiked into every analyzed sample, allowed instrument performance monitoring and aided chromatographic alignment.

The following Tables 6 and 7 describe these Metabolon QC samples and standards employed in the analysis.

TABLE 6

| Type | Description | Purpose |
|---|---|---|
| MTRX | Large pool of human plasma | Assure that all aspects of the |
| | maintained by Metabolon that | Metabolon process are |
| | has been characterized | operating within |
| | extensively. | specifications. |
| CMTRX | Pool created by | Assess the effect of a non- |
| | taking a small | plasma matrix on the |
| | aliquot from every | Metabolon process and |
| | customer sample. | distinguish biological |
| | | variability from process |
| | | variability. |
| PRCS | Aliquot of ultra- | Process Blank used to |
| | pure water | assess the contribution to |
| | | compound signals from the |
| | | process. |
| SOLV | Aliquot of solvents | Solvent Blank used to |
| | used in extraction. | segregate contamination |
| | | sources in the extraction. |

TABLE 7

| Type | Description | Purpose |
|---|---|---|
| RS | Recovery | Assess variability and verify |
| | Standard | performance of extraction |
| | | and instrumentation. |
| IS | Internal | Assess variability and |
| | Standard | performance of instrument. |

Instrument variability was determined by calculating the median relative standard deviation (RSD) for the standards that were added to each sample prior to injection into the mass spectrometers. Overall process variability was determined by calculating the median RSD for all endogenous metabolites (i.e., non-instrument standards) present in 100% of the pooled matrix samples. Experimental samples were randomized across the platform run with QC samples spaced evenly among the injections, as outlined in FIG. 1. FIG. 1 shows the preparation of client-specific technical replicates wherein a small aliquot of each client sample (colored cylinders) is pooled to create a CMTRX technical replicate sample (multi-colored cylinder), which is then injected periodically throughout the platform run. Variability among consistently detected biochemicals can be used to calculate an estimate of overall process and platform variability.

Ultrahigh Performance Liquid Chromatography-Tandem Mass Spectroscopy (UPLC-MS/MS)

All methods utilized a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in solvents compatible to each of the four methods. Each reconstitution solvent contained a series of standards at fixed concentrations to ensure injection and chromatographic consistency. One aliquot was analyzed using acidic positive ion conditions, chromatographically optimized for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot was also analyzed using acidic positive ion conditions, however it was chromatographically optimized for more hydrophobic compounds. In this method, the extract was gradient eluted from the same afore mentioned C18 column using methanol, acetonitrile, water, 0.05% PFPA and 0.01% FA and was operated at an overall higher organic content. Another aliquot was analyzed using basic negative ion optimized conditions using a separate dedicated C18 column. The basic extracts were gradient eluted from the column using methanol and water, however with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternated between MS and data-dependent MSn scans using dynamic exclusion. The scan range varied slighted between methods but covered 70-1000 m/z. Raw data files are archived and extracted as described below.

Data Extraction and Compound Identification

Raw data was extracted, peak-identified and QC processed using Metabolon's hardware and software. Compounds were identified by comparison to library entries of purified standards or recurrent unknown entities. Metabolon maintains a library based on authenticated standards that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library +/−10 ppm, and the MS/MS forward and reverse scores between the experimental data and authentic standards. The MS/MS scores are based on a comparison of the ions present in the experimental spectrum to the ions present in the library spectrum. While there may be similarities between these molecules based on one of these factors, the use of all three data points can be utilized to distinguish and differentiate biochemicals. More than 3300 commercially available purified standard compounds have been acquired and registered for analysis on all platforms for determination of their analytical characteristics. Additional mass spectral entries have been created for structurally unnamed biochemicals, which have been identified by virtue of their recurrent nature (both chromatographic and mass spectral). These compounds have the potential to be identified by future acquisition of a matching purified standard or by classical structural analysis.

Curation

A variety of curation procedures were carried out to ensure that a high-quality data set was made available for statistical analysis and data interpretation. The QC and curation processes were designed to ensure accurate and consistent identification of true chemical entities, and to remove those representing system artifacts, mis-assignments, and background noise. Library matches for each compound were checked for each sample and corrected if necessary.

Metabolite Quantification and Data Normalization

Peaks were quantified using area-under-the-curve. For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”. For studies that did not require more than one day of analysis, no normalization is necessary, other than for purposes of data visualization. In certain instances, biochemical data may have been normalized to an additional factor (e.g., cell counts, total protein as determined by Bradford assay, osmolality, etc.) to account for differences in metabolite levels due to differences in the amount of material present in each sample.

Lipidomic Analysis on Plasma

Metabolon, Inc., Morrisville, USA performed the lipidomic analysis. Lipids were extracted from plasma in the presence of deuterated internal standards using an automated BUME extraction according to the method of Lofgren et al. [19]. The extracts were dried under nitrogen and reconstituted in ammonium acetate dichloromethane:methanol.

The extracts were transferred to vials for infusion-MS analysis, performed on a Shimadzu LC with nano PEEK tubing and the Sciex SelexIon-5500 QTRAP. The samples were analyzed via both positive and negative mode electrospray. The 5500 QTRAP was operated in MRM mode with a total of more than 1,100 MRMs. Individual lipid species were quantified by taking the ratio of the signal intensity of each target compound to that of its assigned internal standard, then multiplying by the concentration of internal standard added to the sample. Lipid class concentrations were calculated from the sum of all molecular species within a class, and fatty acid compositions were determined by calculating the proportion of each class comprised by individual fatty acids.

Proteomics Analysis on Plasma and PBMCs

Omics Technologies Inc., Columbia, MD, USA performed the proteomic analyses on plasma and PBMCs samples. Plasma samples were depleted of the top 14 high abundance proteins to improve the detectability of low abundance proteins. 11-plex TMT labeling proteomics approach was utilized to reveal quantitative proteomics information across different samples in each batch.

Conventional sample preparation was conducted to generate a peptidome sample from each of the 1200 clinical specimen. Each sample was assigned to a multiplex quantitative proteomics assay, and all multiplex quantitative assays are sharing the same reference sample which is created at the beginning of the project through combining 100 randomly selected disease and control samples to best represent the complete profile of the different proteomes expected to be observed throughout this entire study. Large scale quantitative proteomics assays were performed on the state-of-the-art HPLC and LCMS instrumentations, analytical results and raw data were provided under a further agreement for raw data transfer.

Chemicals and Reagents

TCEP (TCEP (tris-(2-carboxyethyl) phosphine) was purchased from Thermo Scientific (Waltham, Massachusetts, USA). LysC and Trypsin proteases were purchased from Promega (Fitchburg, Wisconsin, USA). C18 Cartridges for sample preparation, and chromatography columns for bRPLC and online HPLC of Triple Quadrupole mass spectrometer were purchased from Waters (Milford, Massachusetts, USA). Acetonitrile was purchased from JT Baker (Phillipsburg, NJ, USA), and formic acid was obtained from EMD Millipore (Billerica, MA, USA). MyPro-Buffer I, MyPro-Buffer II and MyPro-Buffer III were utilized by Omics Technologies Inc (Columbia, MD, USA). All other reagents were purchased from Sigma-Aldrich (St. Louis, Missouri, USA) unless otherwise indicated.

Preparation of the Chromatography Solutions

bRPLC Solvent A contained 10 mM TEABC; bRPLC Solvent B contained 10 mM TEABC, 90%

(vol/vol) Acetonitrile. SRM Mass Spectrometry Solvent A was comprised of water with 0.1% (vol/vol)

Formic Acid; SRM Solvent B was Acetonitrile with 0.1% (vol/vol) Formic Acid.

Plasma Sample Depletion

Top 14 most abundant proteins (Albumin, IgG, α2-Antitrypsin, IgA, IgM, Transferrin, Haptoglobin, a2Macroglobulin, Fibrinogen, Complement C3, al-Acid Glycoprotein (Orosomucoid), HDL (Apolipoproteins A-I and A-II) and LDL (mainly Apolipoprotein B)) in the plasma were depleted using the Seppro IgY14 column systems. Plasma samples were diluted 5× in IgY dilution buffer, filtered (0.22 μm), then injected into IgY LC10 columns attached to an Agilent 1200 HPLC system. The unretained fraction was collected.

Protein Preparation

In-solution depleted plasma samples or PBMC cells were further processed by Omics Technologies Inc. (Columbia, MD, USA) using a modified “Filter Assisted Sample Preparation” (FASP) method. Briefly, protein samples were suspended in 9M UREA were then reduced with 5 mM TCEP at 60° C. for 15 min and reduced cysteines were blocked using 10 mM Iodoacetamide (IAA) at 25° C. for 1 hour. Protein samples (100 μg proteins) were then cleaned using Amicon Filters (Millipore, MA, USA) three times with 9M urea and twice with MyPro-Buffer I (Omics Technologies Inc., MD, USA). Samples were then proteolyzed with trypsin (Promega, WI, USA) for 12 hrs at 37° C. The peptide solution then was acidified by adding 1% trifluoroacetic acid (TFA) and was incubated at room temperature for 15 min. A

Sep-Pak light C18 cartridge (Waters Corporation, MA, USA) was activated by loading 5 mL 100% (vol/vol) acetonitrile and was washed by 3.5 mL 0.1% TFA solution twice. Acidified digested peptide solution was centrifuged at 1,800×g for 5 min, and the supernatant was loaded into the cartridge. To desalt the peptides bound to the cartridge, 1 mL, 3 mL, and 4 mL of 0.1% TFA were used sequentially. To elute the peptides from the cartridge, 2 mL of 40% (vol/vol) acetonitrile with 0.1% TFA was used, and this elution was repeated two more times (for a total of 6 mL of eluate). It was important to ensure that the cartridge had stopped dripping before each sequential wash and elution solution was applied. The eluted peptides were lyophilized overnight and reconstituted in 37 μL of MyPro-Buffer II (Omics Technologies Inc., MD, USA).

Multiplexed TMT Labeling

Digested peptides from each sample in a volume of 37 μl MyPro-Buffer II were labeled using 11-plex TMT reagents. After 2 hrs, labeling reactions were quenched and different quantitative channels belonging to each experiment were pooled and dried to remove organic solvents followed by a reconstitution in 500 ul MyPro-Buffer III (Omics Technologies Inc.). Pooled sample was fractionated on a bRPLC (basic Reverse Phase Liquid Chromatography) column (XBridge BEH C18 Column, 5 μm, 2.1×100 mm) via XBridge BEH C18 Guard Column (Waters Corporation) using an Agilent 1260 Infinity II HPLC system. Peptides in each fraction were dried and re-suspended in 8 μl 0.1% formic acid with 3% acetonitrile for LC-MS/MS analysis.

Liquid Chromatography Mass Spectrometry (LC-MS) Analysis

Peptidome samples were fractionated through a Thermo Scientific™ EASY-nLC 1000™ HPLC system with analytical nanoflow column system including a 2 cm trap column and a 75 μm×20 cm analytical column both packed with SuperB C18 HIFI, 5 μm, 100 Å (Omics Technologies Inc., MD, USA). Eluted samples were ionized through Thermo Scientific EASYSpray™ source analyzed on a Thermo Scientific™ Q Exactive HF-X Mass Spectrometer using FT HCD MS2 fragmentation mode with Orbitrap mass analyzer operated at 45,000 mass resolution. Peptides were electrosprayed through a 15 μm emitter (Omics Technologies Inc., MD, USA) at a 2.0 kV spray voltage. Reversed-phase solvent gradient consisted of 0.1% formic acid with increasing levels of 90% acetonitrile in 0.1% formic acid over a period of 130 minutes. The Q Exactive HF-X instrument was operated to automatically switch between full scan MS and MS/MS acquisition. Survey full scan MS spectra (m/z 350-1800) was acquired in the Orbitrap with 45,000 resolution after accumulation of ions to a target value based on predictive AGC from the previous full scan. Intense multiply-charged ions (z≥2) were sequentially isolated and fragmented in the Axial Higher energy Collision-induced Dissociation (HCD) cell using normalized HCD collision energy at 30% with an AGC target 1e⁵ and a maximal injection time of 400 ms at 45,000 resolution.

MS Data Analysis

Mass spectrometry raw files were processed through MyProt-ID pipeline (Omics Technologies Inc., MD, USA) developed for Peptide Identification and Quantification services. Search parameter included oxidation on methionine, deamidation on residues N and Q as different variable modifications. Mass tolerances on precursor and fragment masses were set to 15 ppm and 0.03 Da, respectively. Peptide validator node was used for peptide validation with stringent cut-off of 0.01 and relaxed cut-off of 0.05 (False Discover Rate).

Western Blot of Biomarker Proteins

For protein expression analysis, PBMCs and plasma samples were subjected to two different pre-processing procedures to extract or enrich the proteins, respectively. PBMCs. were lysed with RIPA buffer (1% Nonidet P-40 in 50 mmTris-HCl, pH 7.5, 150 mm NaCl, 0.1% sodium deoxycholate, 1% protease inhibitor cocktails), incubated in ice for 30 min, and then centrifuged at 13 000×g for 15 minutes at 4° C. The supernatant was collected, and the total amount of proteins was determined with BCA assay (Pierce), following the manufacturer's instructions. Plasma samples (10 ul) were depleted from the abundant proteins using the Multiple Affinity Removal Spin Cartridge Human 14 (Agilent), following manufacturer's instructions. Proteins from PBMCs and plasma (40 ug) were resolved on a 10% SDS-PAGE gel and subsequently transferred onto nitrocellulose membranes by electroblotting. The total amount of transferred proteins, used for the normalization of detected proteins, was determined with the REVERT Total Protein Stain kit (LI-COR Biotechnology), following manufacturer's instructions. Membranes were incubated with blocking buffer (TBS: 10 mm Tris-HCl, pH 7.4,150 mm NaCl, 0.1% (v/v) Tween 20 containing 5% (w/v) bovine serum albumin) for 1 hour at room temperature, and then the primary antibodies were added and incubated overnight at 4° C. in the blocking buffer. The following primary antibodies were used: anti-EPX (Thermo Fisher, cat. PA5-42062), anti-ALDH3B1 (Origene, cat. TA811463), anti-SLC25A5 (Thermo Fisher, cat. PA5-90592), anti-AP2S1 (Abnova, cat. H00001175-M01) for PBMCs, and anti-FN1, anti-PRG4, anti-ITIH3 The membranes were washed three times with TBS-Tween 20 for 10 min and then incubated for 2 h with the appropriate secondary antibody. The secondary antibodies used were: anti-mouse HRP conjugated(Amersham), anti-rabbit HRP conjugated (Amersham), IRDye 800CW anti-mouse (Licor), IRDye 800CW anti-rabbit (Licor), IRDye 680CW anti-mouse (Licor), and IRDye 680CW anti-rabbit (Licor). After three washes with TBS-Tween 20, proteins were detected with an ECL detection kit (Cyanagen) or with infrared acquisition at the proper wavelength with the LI-COR Odyssey Infrared Imaging System (LI-COR Biotechnology).

Statistical Analysis and Biomarker Identification

Proteomics analysis (in plasma of n=591 individuals and in PBMCs n=587 individuals), lipidomics analysis (in plasma of n=600 individuals) and metabolomics analysis (in plasma of n=600 individuals) yielded a common set of 585 individuals where proteomics, lipidomics and metabolomics measurements were available.

To identify a multi-omics biomarker signature for the diagnosis of Brugada Syndrome in a human being, a multi-steps procedure has been adopted as follows.

Step 1: within each omics dataset, the features (i.e., proteins, metabolites, lipids) that are significantly different (i.e., differentially expressed (DE) features) between Brugada Syndrome patients and controls have been identified.

Step 2: all DE features (from Step 1) have been combined in a single multi-omics dataset, including proteins (PBMCs and plasma), metabolites (plasma) and lipids (plasma).

Step 3: using the single multi-omics dataset (from Step 2), 6 methods to independently predict features (i.e., proteins, metabolites, lipids) for the diagnosis of Brugada Syndrome have been employed and rank each feature on the basis of its relative “importance” and “frequency” for each method.

Step 4: using the features prediction and ranking (from Step 3), features that are not consistently predicted by each method have been removed, and then the “importance” and “frequency” ranks of the consistently-detected features across all methods have been combined, to derive a single rank (“importance” and “frequency”) for each feature.

This multi-step procedure (above) has been carried out separately in
1) the whole patient cohort (585 individuals comprising 294 Brugada Syndrome patients and 291 controls)
2) the patient cohort of males only (371 individuals comprising 202 Brugada Syndrome patients and 169 controls)
3) the patient cohort of females only (214 individuals comprising 92 Brugada Syndrome patients and 122 controls).

The predictions of the biomarker features in the whole patient cohort, males and females were overlapping, yet not identical. Thus, the biomarker features found in the whole patient cohort and in the male and female cohorts have been cross-matched to identify a common set of features, referred to as “core-set”, for the diagnosis of Brugada Syndrome. The core-set included features for the diagnosis of Brugada Syndrome in males and females as well as in the whole cohort. A larger, extended-set of features for the diagnosis of Brugada Syndrome, referred to as “extended-set” was identified, as those features that were identified in the whole patient cohort. Technical details of each step are as follows.

Step 1. Proteomics data analysis. The whole PBMC and plasma proteomics PSM (peptide-spectrum match score) data matrixes (˜17 k proteins each) were culled for removing species with more than 50% missing values. They were processed with the DEqMS tool in R [30], and the missing values (following a missing-at-random [MAR] pattern) were imputed using a k-nearest neighbour function, and the resulting matrices were normalized using an Equal Median Normalization function. The normalized matrixes were used to identify the DE proteins, using a Spectral Count eBayes function to correct bias of variance estimate based on minimum number of PSMs per protein used for quantification.

Metabolomics data analysis. The metabolomics matrix was processed using the MetaboDiff tool in R [31]. The number of missing values were rather low across samples (less than 15% and followed a MAR pattern) and were imputed using a k-nearest neighbour function. The imputed matrices were normalized using a Variance Stabilizing Normalization (VSN) function, and the differentially expressed Proteins were identified relying on Student's t-Test statistics, while the false discovery rate (FDR) correction of the p-values for multiple testing was done using the Benjamini-Hochberg (BH) procedure.

Lipidomcs data analysis. The lipidomics matrix was processed using the lipidr tool in R [32]. The low number of missing values following a MAR pattern was inferred with a KNN function, and a VSN normalization method was used to deal with the high percentage of low abundance lipids. The differential abundance of lipids between Brugada patients and controls was identified using the limma package [33], based on t-statistics and relying on the for BH procedure for p-value correction for multiple testing.

For each DE analysis of the proteomics, metabolomics and lipidomics dataset, the effect of gender, age, batch and protocol was accounted for and corrected using linear modelling in limma package [33]. The number of DE features (FDR≤5%) identified in each dataset are as follows: 995 DE proteins in PBMCs; 25 DE proteins in plasma; 89 DE metabolites in plasma (44 Xenobiotics and unannotated metabolites were removed); 230 DE lipids in plasma.

Step 2: the DE proteins, lipids and metabolites from Step 1 are combined in a single dataset. This defined a set of 1,339 proteins/metabolites/lipids DE in Brugada Syndrome and which are measured in 585 patients. This data matrix 1,339 features (proteins/metabolites/lipids)×585 (individuals) is used as input for the biomarker prediction in the next step.

Step 3: After the data matrix is autoscaled, the following 6 methods have been used: (1) receiver operating characteristic curve (ROC curve) to determine the AUROC (Area Under the Receiver Operating Characteristics) or AUC (Area Under The Curve) for the prediction of Brugada Syndrome; (2) P-value for differential protein/metabolite/lipid abundance between Brugada Syndrome patients and controls; (3) support vector machine (SVM) with linear kernel [34]; (4) Random Forest (RF) [35]; (5) partial least squares discriminant analysis (PLS-DA) [36], (6) sparse partial least squares discriminant analysis (sPLS-DA). For each feature, univariate AUC and P-value provide a quantification of the feature importance whereas the multivariate Machine Learning (ML) methods, SVM, RF and PLS-DA, provide a quantification of the feature “importance” and of the feature “frequency” (i.e., 100 runs of Monte-Carlo cross validation (MCCV) using balanced sub-sampling where in each MCCV two thirds (⅔) of the samples are used to evaluate each feature importance and frequency). Multivariate sPLS-DA is employed to provide simultaneous feature selection and dimension reduction [37], therefore reducing the number of informative features for the diagnosis of Brugada Syndrome and limiting overfitting. For PLS-DA and sPLS-DA the optimal number of significant components has been determined using R2 and Q2 [38].

Step 4: Biomarker predictions using AUC, P-value, SVM, RF, PLS-DA and sPLS-DA provided a set of 330 features commonly detected by all methods. Each feature is assigned a rank for its relative “importance” (AUC, P-value, SVM, RF, PLS-DA) and “frequency” (SVM, RF, PLS-DA), and we also require the feature to be found in the “sparse” feature selection model by sPLS-DA (for details on how sparse PLS regression achieves both high predictive power and accuracy for finding the relevant features please refer to [37]. An additional requirement is that each feature has some predictive power by its own, and therefore an additional filtering, requiring AUC>0.6 has been adopted. Using the common features predicted by all methods, imposing AUC>0.6 and detected in the “sparse” feature selection model by sPLA-DA, 16 features for the diagnosis of Brugada Syndrome in the whole patient cohort (585 individuals) were identified. Similarly, analysis of the female patient cohort (214 individuals) identified 29 features and analysis of the male patient cohort (371 individuals) identified 28 features. The set of features in common between the whole patient cohort and both female and male patient cohorts consists of 10 features (core-set), while the set features identified in whole patient cohort consists of 16 features (extended-set) which includes all features in the core-set, as reported in the following Table 8:

TABLE 8

| | Cell | Feature | | Overall | | |
|---|---|---|---|---|---|---|
| Signature¹ | type | Name | Description | rank² | AUC | −Log₁₀P |
| core #1 | Plasma | FN1 | fibronectin 1 | 2.78 | 0.651 | 13.3 |
| core #2 | PBMC | AP2S1 | adaptor | 7.45 | 0.616 | 6.2 |
| | | | related | | | |
| | | | protein | | | |
| | | | complex 2 | | | |
| | | | subunit sigma | | | |
| | | | 1 | | | |
| core #3 | Plasma | ITIH3 | inter-alpha- | 8.62 | 0.602 | 4.5 |
| | | | trypsin | | | |
| | | | inhibitor | | | |
| | | | heavy chain 3 | | | |
| core #4 | PBMC | EPX | eosinophil | 8.66 | 0.624 | 6.5 |
| | | | peroxidase | | | |
| core #5 | Plasma | fumarate | fumaric acid | 9.40 | 0.632 | 6.2 |
| extended #1 | PBMC | ALDH3B1 | aldehyde | 9.97 | 0.633 | 6.3 |
| | | | dehydrogenase | | | |
| | | | 3 family | | | |
| | | | member B1 | | | |
| core #6 | Plasma | PRG4 | proteoglycan | 10.33 | 0.620 | 6.2 |
| | | | 4 | | | |
| extended #2 | PBMC | SLC25A5 | solute carrier | 10.49 | 0.620 | 6.8 |
| | | | family 25 | | | |
| | | | member 5 | | | |
| extended #3 | Plasma | PON1 | paraoxonase | 10.77 | 0.604 | 6.2 |
| | | | 1 | | | |
| core #7 | PBMC | COG5 | component of | 12.25 | 0.601 | 5.2 |
| | | | oligomeric | | | |
| | | | Golgi | | | |
| | | | complex 5 | | | |
| extended #4 | Plasma | 2- | alpha- | 12.72 | 0.635 | 6.7 |
| | | hydroxydecanoate | hydroxycapric | | | |
| | | | acid | | | |
| core #8 | PBMC | RIOX1 | ribosomal | 13.42 | 0.612 | 4.5 |
| | | | oxygenase 1 | | | |
| core #9 | Plasma | glutamate | L-Glutamic | 15.00 | 0.613 | 6.6 |
| | | | acid | | | |
| core #10 | Plasma | Palmitoleoylcarnitine | O- | 15.60 | 0.607 | 5.0 |
| | | (C16:1)* | palmitoleoylcarnitine | | | |
| extended #5 | PBMC | S100A11 | S100 calcium | 16.14 | 0.612 | 5.6 |
| | | | binding | | | |
| | | | protein A11 | | | |
| extended #6 | PBMC | PLCB2 | phospholipase | 17.08 | 0.601 | 4.2 |
| | | | C beta 2 | | | |

¹core: features in the “core-set” (n = 10) identified in males and females as well as in the whole patient cohort; extended: features in the “extended-set” (n = 6) that have been identified in the whole patient cohort.
²Features are ranked according to the overall rank obtained from the analysis of the whole patient cohort (n = 585 individuals), which combines (adds up) the feature's rank calculated based on the feature relative “frequency” and “importance” across AUC, P-value, SVM, RF and PLS-DA methods.

### Example 3: Identification of Deleterious Mutations in Protein Biomarkers of the Invention

Materials and Methods

Genetic analysis in a subgroup of Brugada Syndrome patients (n=186) by whole-genome sequencing (WGS) by both PolyPhen [39] and/or SIFT [40] has been carried out.

SIFT predicts whether an amino acid substitution is likely to affect protein function based on sequence homology and the physical-chemical similarity between the alternate amino acids. The data provided for each amino acid substitution is a score and a qualitative prediction (either ‘tolerated’ or ‘deleterious’). The score is the normalized probability that the amino acid change is tolerated so scores nearer zero are more likely to be deleterious. The qualitative prediction is derived from this score such that substitutions with a score<0.05 are called ‘deleterious’ and all others are called ‘tolerated.

PolyPhen-2 predicts the effect of an amino acid substitution on the structure and function of a protein using sequence homology, Pfam annotations, 3D structures from PDB where available, and several other databases and tools (including DSSP, ncoils etc.). As with SIFT, for each amino acid substitution where it was possible to calculate a prediction, both a qualitative prediction (one of ‘probably damaging’, ‘possibly damaging’, ‘benign’ or ‘unknown’) and a score were provided. The PolyPhen score represents the probability that a substitution is damaging, so values close to one are more confidently predicted to be deleterious. The qualitative prediction is based on the False Positive Rate of the classifier model used to make the predictions.

Results

Many Brugada Syndrome patients carry a significantly high number of deleterious mutations even when compared with the SCN5A gene in which mutations are most commonly found in Brugada Syndrome [10, 11].

Specifically, the genes encoding for the biomarker proteins: EPX (eosinophil peroxidase), PON1 (paraoxonase 1) and PRG4 (Proteoglycan 4) carry the same mutation in 91 patients, 110 patients and 123 patients with mutations, respectively, as shown in FIG. 8.

The most highly mutated gene is Proteoglycan 4 (PRG4), a member of the proteoglycan family, which plays a diverse range of physiological and pathological mechanisms including metabolic pathologies [41]. Proteoglycan 4 is ubiquitously expressed and secreted into the circulation and has emerged from human association studies, on both transcriptional and protein levels, as a possible factor contributing to weight gain, dyslipidaemia and insulin resistance and PRG4 plasma levels were found to be associated with obesity and weight loss [42-43]. Mouse studies showed a causal role for PRG4 in the regulation of energy metabolism [44] and given the upregulation of PRG4 in obese individuals with type 2 diabetes, it can be suggested that PRG4 also plays a causal role in the development of metabolic disturbances in humans.

Another highly mutated gene encoding for dysregulated biomarker protein is paraoxonase 1 (PON1), a liver-derived glycoprotein that is secreted into the circulation and is associated with high-density lipoprotein (HDL) in the blood. PON1 is considered to play a crucial role as an anti-atherosclerotic factor. This enzyme exhibits antioxidant and antiatherogenic properties and, furthermore, alterations in circulating PON1 activity have been associated with a variety of diseases, including diabetes mellitus, obesity, hepatic and renal diseases, psoriasis, cancer, and rheumatoid arthritis [45]. Therefore, it has been also suggested that PON1 concentration and activity be used as a biomarker for the detection of various diseases [46]. However, the activity of PON1 is both under genetic and environmental regulation (including age, sex and lifestyle, and pharmaceutical interventions), and it has been shown to vary widely between individuals [47]. The very high number of mutations (>100) detected in PON1 in Brugada Syndrome patients suggests a causative role for this gene in the Brugada Syndrome disease, and possibly a functional role for oxidative stress control, as shown for other diseases like breast cancer in diabetic patients [48].

The following Table 9 reports the sequence variants for the most highly mutated genes (PRG4 sequence variant: 1-186304862 C→T, EPX sequence variant: 17-58193733 G→C and PON1 sequence variant: 7-95316772 A→T). All details about the deleterious mutation were detected in the Brugada Syndrome patients: chromosome (Chr.), DNA strand (Strand), genomic position, reference and mutated allele, codon and amino acid changes.

TABLE 9

| | | | | | | | | | N. of |
|---|---|---|---|---|---|---|---|---|---|
| | Gene | Variant | | | | | Codon | Aa | Brugada |
| | Ensemble | name | | Position | Ref | Mut | change | change | patients |
| Gene | ID | GRCh38 | Chr. | (bp) | allele | allele | (ref/mut) | (ref/mut) | with |
| PRG4 | ENSG00000116690 | 1- | 1 | 186, 304, | C | T | Cgg/Tgg | R/W | 123 |
| | | 186304862 C-T | | 862 | | | | | |
| EPX | ENSG00000121053 | 17- | 17 | 58, 193, | G | C | caG/caC | Q/H | 91 |
| | | 58193733 G-C | | 733 | | | | | |
| PON1 | ENSG00000005421 | 7- | 7 | 95, 316, | A | T | Atg/Ttg | L/M | 110 |
| | | 95316772 A-T | | 772 | | | | | |

Finally, frequency of the mutation in Brugada patients cohort as well as in reference European and African American populations has been detected and reported in the following Table 10. Population frequencies for the mutated allele have been retrieved from The Genome Aggregation Database (gnomAD) database v3.1 (https://gnomad.broadinstitute.org/).

TABLE 10
Mutation frequency

| | Brugada | | African/African |
|---|---|---|---|
| | patient | European | American |
| Gene | cohort | population | population |
| PRG4 | 0.3918 | 0.3439 | 0.1864 |
| EPX | 0.2756 | 0.3256 | 0.2503 |
| PON1 | 0.3567 | 0.3660 | 0.1749 |

### REFERENCES

[1] Antzelevitch, C., et al. Circulation, 2005. 111(5): p. 659-70.
[2] Brugada, P. and J. Brugada. J Am Coll Cardiol, 1992. 20(6): p. 1391-6.
[3] Nademanee, K., et al. J Am Coll Cardiol, 2015. 66(18): p. 1976-86.
[4] Priori, S. G., et al. Eur Heart J, 2015. 36(41): p. 2793-867.
[5] Mellor, G., et al. Circ Arrhythm Electrophysiol, 2014. 7(6): p. 1078-83.
[6] Nademanee, K., et al. Circulation, 1997. 96(8): p. 2595-600.
[7] Tan, H. L., et al. Circulation, 2005. 112(2): p. 207-13.
[8] Pappone, C., et al. J Am Coll Cardiol, 2018. 71(15): p. 1631-1646.
[9] Chen, Q., et al. Nature, 1998. 392(6673): p. 293-6.
[10] Kapplinger, J. D., et al. Heart Rhythm, 2010. 7(1): p. 33-46.
[11] Antzelevitch, C. and B. Patocskai. Curr Probl Cardiol, 2016. 41(1): p. 7-57.
[12] Mango, R., et al. Circ J, 2016. 80(4): p. 938-49.
[13] Pappone, C., M. Monasky, and G. Ciconte. Eur Heart J Suppl, 2019. 21(Suppl B): p. B61-B66.
[14] Yar, S., M. M. Monasky, and R. J. Solaro. Pflugers Arch, 2014. 466(6): p. 1189-97.
[15] Monasky, M. M., et al. Front Physiol, 2017. 8: p. 1056.
[16] Monasky, M. M., et al. Front Physiol, 2018. 9: p. 1088.
[17] Letsas, K. P., et al. Pacing Clin Electrophysiol, 2017. 40(12): p. 1332-1345.
[18] Hofman, N., et al. Circulation, 2013. 128(14): p. 1513-21.
[19] Lofgren et al. J Lipid Res 2012; 53(8): p. 1690-700.
[20] Casillas-Ituarte, N. N., et al. J Biol Chem, 2012. 287(9): p. 6693-701.
[21] Schumann, T., et al. Pharmacol Rev, 2020. 72(1): p. 343-379.
[22] Shoshan-Barmatz, V., A. Shteinfer-Kuzmine, and A. Verma. Biomolecules, 2020. 10(11).
[23] Rhee, S. G. Annu Rev Biochem, 2001. 70: p. 281-312.
[24] Hwang, H. J., et al. FASEB J, 2019. 33(10): p. 10668-10679.
[25] Marchitti, S. A., et al. Free Radic Biol Med, 2010. 49(9): p. 1432-43.
[26] Jin, M., et al. Cell Death Dis, 2013. 4: p. e554.
[27] Wishart, D. S., et al. Nucleic Acids Res, 2018. 46(D1): p. D608-D617.
[28] Szklarczyk, D., et al. Nucleic Acids Res, 2016. 44(D1): p. D380-4.
[29] van Karnebeek, C. D. M., et al. Am J Hum Genet, 2019. 105(3): p. 534-548.
[30] Zhu, Y., et al. Mol Cell Proteomics, 2020. 19(6): p. 1047-1057.
[31] Mock, A., et al. Bioinformatics, 2018. 34(19): p. 3417-3418.
[32] Mohamed, A., J. Molendijk, and M. M. Hill. J Proteome Res, 2020. 19(7): p. 2890-2897.
[33] Ritchie, M. E., et al. Nucleic Acids Res, 2015. 43(7): p. e47.
[34] Barla, A., et al. Brief Bioinform, 2008. 9(2): p. 119-28.
[35] Breiman, L. Machine Learning, 2001. 45(1): p. 5-32.
[36] Xia, J. and D. S. Wishart. Curr Protoc Bioinformatics, 2016. 55: p. 14 10 1-14 1091.
[37] Chun, H. and S. Keles. J R Stat Soc Series B Stat Methodol, 2010. 72(1): p. 3-25.
[38] Triba, M. N., et al. Mol Biosyst, 2015. 11(1): p. 13-9.
[39] Adzhubei, I. A., et al. Nat Methods, 2010. 7(4): p. 248-9.
[40] Sim, N. L., et al. Nucleic Acids Res, 2012. 40(Web Server issue): p. W452-7.
[41] Bishop, J. R., M. Schuksz, and J. D. Esko. Nature, 2007. 446(7139): p. 1030-7.
[42] Geyer, P. E., et al. Mol Syst Biol, 2016. 12(12): p. 901.
[43] Oller Moreno, S., et al. Proteomics Clin Appl, 2018. 12(1).
[44] Nahon, J. E., et al. Biochim Biophys Acta Mol Basis Dis, 2019. 1865(2): p. 494-501.
[45] Costa, L. G., G. Giordano, and C. E. Furlong. Biochem Pharmacol, 2011. 81(3): p. 337-44.
[46] Camps, J., J. Marsillach, and J. Joven. Clin Chim Acta, 2007. 386(1-2): p. 114-5.
[47] Zhao, Y., et al. Mol Genet Metab, 2012. 105(1): p. 141-8.
[48] Eraldemir, F. C., et al. J Med Biochem, 2019. 38(3): p. 368-375.

1. A set of biomarkers comprising:
i) a subset comprising the following proteins:

| Protein | UniProtKB ID |
|---|---|
| Fibronectin 1 (FN1) | P02751 |
| Inter-alpha-trypsin inhibitor heavy | Q06033 |
| chain 3 (ITIH3) | |
| Proteoglycan 4 (PRG4) | Q92954 |
| Adaptor related protein complex 2 | M0QYZ2 |
| subunit sigma 1 (AP2S1) | |
| Component of oligomeric Golgi | Q9UP83 |
| complex 5 (COG5) | |
| Eosinophil peroxidase (EPX) | P11678 |
| Ribosomal oxygenase 1 (RIOX1) | Q9H6W3 |

and
ii) a subset of comprising the following metabolites:
fumaric acid;
O-palmitoleoylcarnitine;
L-Glutamic acid;
for the diagnosis of Brugada Syndrome in a human being.

2. The set of biomarkers according to claim 1, further comprising one or more of the following additional biomarkers:
i) a protein selected from the group consisting of:

| Protein | UniProtKB ID |
|---|---|
| Paraoxonase 1 (PON1) | P27169 |
| Aldehyde dehydrogenase 3 | P43353 |
| family member B1 (ALDH3B1) | |
| Solute carrier family | P05141 |
| 25 member (SLC25A5) | |
| S100 calcium binding | P31949 |
| protein A11 (S100A11) | |
| Phospholipase C beta | Q00722 |
| 2 (PLCB2) | |

and/or metabolite 2-hydroxydecanoate;
and combinations thereof,
for the diagnosis of Brugada Syndrome in a human being.

3. The set of biomarkers according to claim 2, further comprising:
i) a subset of the following proteins:

| Protein | UniProtKB ID |
|---|---|
| Fibronectin 1 (FN1) | P02751 |
| Inter-alpha-trypsin inhibitor heavy | Q06033 |
| chain 3 (ITIH3) | |
| Proteoglycan 4 (PRG4) | Q92954 |
| Adaptor related protein complex 2 | M0QYZ2 |
| subunit sigma 1 (AP2S1) | |
| Component of oligomeric Golgi | Q9UP83 |
| complex 5 (COG5) | |
| Eosinophil peroxidase (EPX) | P11678 |
| Ribosomal oxygenase 1 (RIOX1) | Q9H6W3 |

and
ii) a subset of the following metabolites:
fumaric acid;
O-palmitoleoylcarnitine;
L-Glutamic acid;
2-hydroxydecanoate;
and combinations thereof,
for the diagnosis of Brugada Syndrome in a human being.

4. An in vitro method for detecting a presence and/or measuring concentration of an occurrence of one of the set of biomarkers according to claim 1 in a biological sample of a human being.

5. An in vitro method for detecting the presence of one of the set of biomarkers according to claim 4, comprising:
a) detecting the presence of at least 7 up to 12 proteins of subset i) by an assay selected from the group consisting of Western Blot, Dot blot, ELISA, flow cytometry, enzymatic activity assay, targeted MS, multiplex protein analysis, ProQuantum high-sensitivity immunoassay, and combinations thereof;
b) detecting at least 3 up to 4 of the metabolites of subset ii) by an assay selected from the group comprising MS, GC-MS, fluorimetric or colorimetric assays, NMR, spectroscopy with nanoprobes, Enzyme-Linked oligonucleotide assays, and combinations thereof;
wherein a positive detection of all the biomarkers compared to a normal control allows the identification of patients affected by Brugada Syndrome.

6. An in vitro method for measuring the concentration of one of the set of biomarkers according to claim 4, comprising:
a) quantitatively determining at least 7 up to 12 proteins of subset i) by a quantitative technique taken from the group consisting of Western Blot, ELISA, enzymatic activity assay, targeted MS, multiplex protein analysis, ProQuantum high-sensitivity immunoassays, and combinations thereof;
b) quantitatively determining at least 3 up to 4 of the metabolites of subset ii) by a technique taken from the group consisting of HPLC, MS, NMR analysis, ELONA, and combinations thereof;
wherein an increase or decrease of the concentration of the biomarkers compared to a normal control indicates a diagnosis of Brugada Syndrome.

7. The in vitro method according to claim 4, wherein the biological sample is selected from the group consisting of plasma, PBMCs, whole blood, serum and peripheral blood, or a combination thereof.

8. The in vitro method according to claim 4, wherein the human being is asymptomatic.

9. The in vitro method according to claim 4, wherein the human being is at high risk for Brugada Syndrome due to family history, previous events of heart atrial and/or ventricular fibrillation, diabetes or obesity.

10. The in vitro method according to claim 4, wherein the human being is about 40 years old or younger.

11. A kit comprising antibodies specific for each of the proteins of subset i) according to claim 1, wherein said antibodies are labelled or attached to a solid support.

12. An oligonucleotide sequence of Prg4 gene (ENSG00000116690) characterized by the presence of a deleterious mutation C→T in position 186,304,862 of the nucleotide sequence, said oligonucleotide sequence being RNA or DNA, for use as a genetic diagnostic marker for the diagnosis of Brugada Syndrome in a human being.

13. A mutated PRG4 protein encoded by the oligonucleotide sequence according to claim 12, for use as a genetic diagnostic marker for the diagnosis of Brugada Syndrome in a human being.

14. An oligonucleotide sequence of Epx human gene (ENSG00000121053) characterized by the presence of a deleterious mutation G→C in position 58,193,733 of the gene sequence, said oligonucleotide sequence being RNA or DNA, for use as a genetic diagnostic marker for the diagnosis of Brugada Syndrome in a human being.

15. A mutated EPX protein encoded by the oligonucleotide sequence according to claim 14, for use as a genetic diagnostic marker for the diagnosis of Brugada Syndrome in a human being.

16. An oligonucleotide sequence of Pon1 human gene (ENSG00000005421) characterized by the presence of a deleterious mutation A→T in position 95,316,772 of the gene sequence, said oligonucleotide sequence being RNA or DNA, for use as a genetic diagnostic marker for the diagnosis of Brugada Syndrome in a human being.

17. A mutated PON1 protein encoded by the oligonucleotide sequence according to claim 16, for use as a genetic diagnostic marker for the diagnosis of Brugada Syndrome in a human being.

18. A method for the diagnosis of Brugada Syndrome in a human being comprising detecting in a biological sample one or more of the oligonucleotide sequence or mutated protein according to claim 12, wherein said biological sample is selected from the group consisting of plasma, PBMCs, whole blood, serum and peripheral blood, and combinations thereof.

19. An in vitro method for detecting the presence of one or more of the oligonucleotide sequences according to claim 12, comprising conducting by genetic analysis through PCR or DNA sequencing.

20. A kit comprising a primer or probes complementary to one or more of the oligonucleotide sequences according to claim 12, for the diagnosis of Brugada Syndrome in a human being.


SEQUENCE LISTING (9 sequences)

SEQ ID NO: 1 - DNA, 19489 bp, Artificial sequence
  Prg4 human gene
taaaggaaggatctaaggaaaatatcaatacattttttaagcagatttttaaaataataaaggaagctagaaaatccagtgtcaataatctacctgtaggtaactgagagcatgttttctgtgctcagagaaaatgaaactactcaagaaatgtatatttgatataatggtataagtatataaaatgtatgccacaagctaacactaggtaaatttcaaagctacctttgctagatttaactggaaagcagaagaaataaatgataccagtatgtgaatattttaaagttgctgcttcaatatactagtcaaagtctgccagagctatttaccatctcaggacaatttgttcacttcacttagaaattagaaatgtgctctggccaatacatctaaatttattttgtggtagtcattttgcttaatggaagtgttttccagatgttgcctaagtctagtcgtctgggcccttttccaataaatgaggaaagtttgatttcataggttgtcactgttgattttgtctaacctttggactaattggttcatcccactgtatttgcactgatatataagactcccaggacgtgggataaactcatctatcctttacgggtaagtgtcaaagttaacttgcttcatagaattaaatgtgttttcattagaggtgtttgaaaaaaaatgtgtaaagatagtagttggaaatttttgaaaaggattatgtttatgcaatacacctgttggaagcctttgaatttatattgagaattaagaaaaagttggaacagtaactccatgatgcttattaaattacatttctgtgacacaggttattattttccttaagataaaattttaaccttgcactgttaagtacatgccatactttggctagagttttaagataagtctattctatattggagatttcttaagaattaaaactgtcaataacgtggctgagggtgacttgatttgttctgcataagattaagtctaatggccaaatattttctataaaattaaagctgatttaaagtcttgtttcaagaatgggatggttttattattgttacatttttaaagttactgaaatgtgtataatgcaagcctaagttagtggtgagatgaaagagctgttttctgatacttttattttattttcagcaagggtacctacggtacctgaaaacaacgatggcatggaaaacacttcccatttacctgttgttgctgctgtctgttttcgtgattcagcaagtttcatctcaaggtagcttaaccatcgaacatacttttatttaacaactattgctaatcattcagtcttgatttttataacaacggaaatatatttctaaaaatttatatttgcttgagtttaatatactgtacaaccttaaaataatatataacatgtagcctgtttgtaagtgctttggaattctcatctttttctccttttgagagttttgagcttgtataaagaataattttggtacttgttatttctatggttctctcgaaagtttaaaaagtgcagacagttcatcataaaattatgctggagctatatagtatcataaaaataactaaaaaagaacaatatttagataacatgtaatttttgcctatatgttgaacaatttgttttttaaaatcaaataaattattgataaataacttattttggcatcatttagacatagataccatattataataaactatagtgtttcaaatggcattttattagtcttgaaaatgttctctttttcttacaagaactatttatccaggtatggacatatacagatgtgactgtcattttgtgttataacattaaacttttcacagttctcccaaaacaggtcctggatgaatagttcttgttactcatttttagagactgttaaagtacattcagtgaaatcacattagtaaaagatgctagcatgccatctagggggattaatagtaacaatgccaagctttggatttttcttttcttttcttttccttttgttcccctttgataagcaaaatctgagagaataaaaatcaagattcatgacagttatgatgaaattatgtttctaaagtaaacatttccagtaaaatacgagattcttatgaaacctgaacatgagtggtaactgtctgcataggcataagttgcagaattgtttagataagaaaagacaggaaaacacatgaaagcaaatgtgaatattcaataagaatgatgactactccagtatctccagacccttcggctttctcgtaacactatgacaaggttcacaacactgggggcactttctaaactgccttttcctctgtgatacaattggttgttcactaaaacagtgttactttcattttaattgtgattaaataaatcaaattaaaattaatggggctgggtgtggtggctcacacctgtaatcccagcattctgggaggcctagggagacagatcacctgaggtctggagttcaagagcagcctgggcaacatggcaaaatcttgtctctatacaaaatacaaaaaaattagccaggtgtggtagtgtgtgcctgtaatcccagctattcggaggctgaggcaagaggatatcttgaatccaggaggtggaggttgcattaagctgagcttgcatcatggcactccagcctgggcaacagagtgagactctgtctcaaaaataaataaataaataaataaaattaatggttacaattaatagcaataaaagtaggacagacacctaatctatgaaagtaagcttttcctgtaaggatgatttcctcttttttttttttcttttgagacagggtcttgctttgtctcccagtctggagtgcagtggcacaatcttggctcactgcaacctccacctacggggttcaagcgattctcctgtctcagcctcccgagtagctgggattacaggcccctgccaccatgcccggttaatttttgtatttttagtaaagacaggttttcaccatgttggccaggctggtctcacctgacctcaggtgatccgcccgcctcagcctcccaaagtgctggaattacagatgatttcttatttcagaaatctgccaactataaaagagcaatctcttgatactgtcttgtctgcttctcttgctttctcaacctcttctcattctcttttttctttatatataatatatgtatttatatacatacactatatatacatttttgtatgcattatgcactcatgtacgcaaaaagttctgaaagttgtcctacaatttactgttttatttgcatattcagactttggcattcctggactctattcttttaagatttgttttcagtgtgtttcaacattcctttgtggatttaggacagtacacctgccaatttctattccagggatggattccattgtcacatttctgcagtcatttctcagggagggttttaaggtggtgttttccaaatgacttttaaaaaatatttgagaatgagtatgcttttgttattgtttgcttatttgttgcctggaaaacctctgttcatgacctttatagttatgcccagttattttaagggttctcacttcagcagtggttgtaagcatctgcctcccctgacattttaaaaatccagttatccccaccaattaaggaagaaaaagctcagtaaagtttatgctggtgcaaccagcgtgctttaaacttatcctcaggaatgggaggcaaagcgacaggtggaaacatctcaggcttaaaataagatatatcagagttcaaattctggatggattgtttacttaaggtgaccataaaatgtattatctaaattaggacaattataagggtaaaagagttcactacaaataattgggaggtattaattaacctatgatgccaatatactggaactattaactagtcgtgtgataatgagcaagttgtcaaaatttctgtgtttcagctttcttagctctaaaaaagggctactaaatcctggaaacattttcataagcattagtgataatgtatgactcagcacctggcatagagaagctcaataaatggcaactgctaatcatcaaaaccacagatcggtagtagctgtagctgccacctcttccttaggagtatcaccctctttaggcaaagccattactttgcctggttttcaaaaatgtgtttatcatctcagtctagtgaagaataaagtgacataattgagatcacctaagacatcagccaaatatcagctgggcctattgctgacatcattccaacaccttctcgatcaataaaattctctctcaccaagtggctttgtccccctcgttagattgctccctttctataaagtggtttggccatatttacgccagtattgtataattttagatttatcaagctgtgcagggagatgtggggaagggtattctagagatgccacctgcaactgtgattataactgtcaacactacatggagtgctgccctgatttcaagagagtctgcactgcgggtaagtcctgagagcgggtgtctcctctgtcaagcaacactgcgagtctgtgagtccccccttgcaccctcgtgcagtgctgtgagactgagcctccccttgcacccacttgcagtgctgttttcccactgtatcacttcctttgcttaagtgactttccttcacttgcataaatgttgcagcgcattgtacctaaggatggtgtcaaagcttgtgattattggggagggtaaggggagcctggaagtctgtcacttgcacaaggttattgtctagttccaagtaaatgtcttattttaaatagagaagatactgtgagtcaggagtcattaagtaaatagtaaggagtaattatttacaatcagagatgtcctcgactagtaatgaggactttcaatagtttcatcaaaagaatacatgagcatggaataatcttgtctaccattggactttctgatgttaacacgtttgtaaattacctggaaaagacactttaaagataaagtgctagtgtttaatgatgaatttcagttaaaatctgaaggtctgaattttaaattatgagtggtgtttcacttgaatatattttgggccattttattattaacacagaaacatttcctattcaatttaagaaaattgtaattaaagttatttgaaaggtgtatttgctttagaatttgaatgtgaagttgaggaatatattgtgcaatttatgtattggataggtaattatttcaaataagccttggtaagtccctgttaactctaacaaaggctttttaaattttcattttttaaatgagtagtcctccctaacatagtccacactgtaagattaggctgaaagctttcaactatacaccttccccagtttgtcataataagggcctctgcattacaaatgatttttaagcctcaaaaatgacccatttacgtggaatatatatacatatatatttatatggaatatattgctgagatctgtacattcaattactgtgaatctattacaaagcagtgtgtgaagaggagaaggatgaagagatttcatatgaaggctatctcactatctagacatttcccgatttttctttgtccatacatgtaaataactcgggcagcatcaggatgtctcttggagtctggaagggcaagaggagttgccctcagtcaccatatttcttttttgacttgggctgtctccatctgggataccatctaatttttcctggatgatgtactccaaatttcaaataaaagacttagaaatgaacttttggaaacctagtcaagtctaaggtgggaaatggctgtcaaatacgtgggcctggcttcacaatgaataatctgtaacttcttgttttgctctgggtagagctttcctgtaaaggccgctgctttgagtccttcgagagagggagggagtgtgactgcgacgcccaatgtaagaagtatgacaagtgctgtcccgattatgagagtttctgtgcagaaggtaagcatcacagtaccaaccaatgcttctcagtacagccagatctgtgcacctaccttagcatcactgataatgtctaacacgcagtccatcttaggccttgattttactaacatataagctatttcataataattttgttttgtggagatgaattaaagaagaaaagcgccttccaacagcagaacccccaggctcagccaggctccaagagtacaagatgttgccagagggggaacatagaggatgaggctgaatgtatatgtcttttttttcttcagatatggttcaatttcaggttaaatcaacagtaatcaaatcacttgatgatttcttgtctcacccttgctgacttcacgaagtcaactatcctttgagctatctgtgcttttggcctcagtgtttgagtatatatttaaggacataagcaacactttcagcttccctaaaccagtaaatttatgaattagtttaagccattaaatttataggcaaatctacctaacacgttgttaactgtcatgttccttcccatttctgcagtctcctcagcagtctctattccaatacatgtgcatggtaaagtcaataacaagggatattcccatcttaaaatcaaattctctatttataggtcttgaaggactcctctagagtataacaaaaggctccagattttacagaaaaagattttaaaaatatacttgaatactggattactttcctttaccaaatggttgttcttaaattctactttgttaaaacaagaatatttgtggaggtaaaagtgaaaataagctttgcttgcataatcaatcttatcattttatttaaacaagcctgcgcttcctgcttctttagaacagtgaagaaagatcggaaaactgctaatcagcagaattgagagatgtttcttaaccgttcaatgttttcatgggaaaacattccaaaaatgatgaagacaacacccttgtaataaaaaccttagagctcacccttcctgcagattttagcaggccccatgatgagtgacacatatttactcattaccctaattgttttggctgtgagacaagaaagctaagaatttgttttgggcagaggatgtctagggagtgaagatggttagaaagtgatgagacattgagagaacattttgataaagataatactttatgttatgaaatatttggtcagattacctaaagtttaggtagaactctgaaaaaaaaatgatgaaggcgatgctggccaactttctttacagaattcataagacagtcagcgtcccaacttaaaataataaaaccactcagaacctcaatacttcattttttaagtgtaataaccaaactatcttgagattcactacttagttgtttcagtatagatactacttacaggtgtctatatatctaaaaatcataacatttcaagaattgaaggaatcccagccatccaagtgcctctgaatgtgaaagagaaaacagagtccaagaggttaaccattttggccaagaacattagctagtacatggcagatctgtttctcaaattgagatacttgatgtttttgctgggtaggaggagaaacaagagggacagtggccagttcacggagactatagcagtagggttctggggcgatgttgcggggagcagcatgatttatcacaacccttctgtacccagagagtccccagtgctgttttcttatccttcattagtccctcttcttgttcctaaaaaaaaaaaaaaaaaagaagttccatgttttctcttctagactgagaagcggaaaagggaaggggactaaaaggttaaaaagatacacaccaaaccattaagttgtatctttgggaagggcagggcattttgaggaaaagcacaatgtgaaggtgtgaacttttcacaagaatattcatgtagaacttttgtaattaaaaaaagaacaaaaggcaaattggcatcatttaatttaaatgaatattaaaacagaaaaaaatgaatactaaaacagaaaaaaattctgaaggcaaagaccttcagaaatcatcttgaataaacatcttagtttgtagacaagaagatgaagttcaagataattatttgcctaaggtcacaatggtagaggctcgcaggcctaggactggaatttaggtcttctgattcctgagactttgctccctgtcataacttacaacaatttgagaaccagtattttatagcaatgcatccttagacttaaatggagtcattcgtgttgagctggagcctgcctgcactggctgtcaccagcatctactcttgaagctagatgcatctgtgcttttcacagttagagctgctgatgaacataaacaagatgttaactgacttgtcttacttggcctcagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaaggtaggaagatgacagatataatcaaaggagctttcttagatgaagtaacttgtaggtgactgcttatctaagcccattctcagagaacagggtaatcttaggaatcatgagcctcattacactcgaaggttttagactttgcttttaagtaaagtttaagacaaagtataaactctcagctctttctgtatttaccaaacccagcatgagtctgggttaaaacaaatcagaagggacaaatcttactataaaaaacaaaaaccaccccatgattttctttgtagaataatttgattctgtgttttggaggatatgggaaagttgagagatactagtaatactgctagtatctgtgataagcccaggtgccttgcttttaactgacagattaaaaggcagttggtcatattactaataaaagcaaaatccagatacttgtagactagtaaatagcacttcttgctgtgtttagactggtggttcttttttgttttaaatcacagttggtgtgatcaaactttctatttgatttatagaacattctgtttctgaaaatcaagagtcctcctcctcctcctcctcttcctcttcttcttcaacaattcggaaaatcaagtcttccaaaaattcagctgctaatagagaattacagaagaaactcaaaggtttgagcattgataaagatcaaagactgtatcaatgccatattaacaatgattatatttaaaagtaatgcgtgttggcagaatgaggacatcttaatatgggggggaatataactttatgaatattatcttagaaagtaccaaatgaatacttcctacaggaaattagtgccaagcggctaagaagccactctaaaagcaaaggataagaagatgctgtagcaggactgtggaaccacaagatggcaccagagatgattattgatggctttctactgtcattcttgtttacttgacatcattttgcttcttctccttcatcttccttgcattttcattttcccttccccagctttattttaacaaacatttatatactattgttctaagtgttttacaagtattaactcattaaattcttacaaacctctatggaaaaaatattatccatattttacaaatcctctttattttcctagtaacttatatattatatctggctgacttttcctctagtagagactgtgctacagaataggctattattgcctgaaaagagatttcctttgctgtttattatttcctgaagttctggttctaaatatatattctctttctccaggaaatactggatttttggataagggagttttaaagaactctaagaggttgtgatttcctccgaagcattcgctctaaataaatagccaggcatttaatcttttagtcacccagaaaatcgtgcaggaagccacccttgggctttgtagtccacatgtggggcagctgctatgggaaagccttgataaatactgtctttttgctgcccatcacaccctaagctcctccttatgtcccctgggacctgcaaggttagctgcgcagaagaaaaggagtggctaatagccctatgtatgggaacaaggctcagcagggactgtgggggtgactggggcagattgctcagaggtagctgtagaggcctctgagataattggtgcagcaagatggtagtactaggtttccaggaggaaggaagggacactgggcactgaagaataagagtacaaaaagtagggggtaaagaacattaatataaacagatgaattagtgttatttatcgtgttggtaattttgaacttgccactgacttccatgtattaattgtatagagttttatttgtgctttttcatccctcatctcatttattccctaaaacagcccaataagataggttgagtatttgccattttacaacaaattaaaggacatagtagagattagcaatttgcttagaaacacacaactagtcaatgataaagatgggatctttatgtcactattaaagaaaaaaaaatattctaaaataacaagatgtatattttttctccagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccactcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccggtattaagaatcagttattttcatttttaaagctggtaatgttagtttacatctatctgaaccagaatgccttagtttagtatcactcaactgagatatattacctgaccttgttagcttgtgaagttttacagcttccctgtaggtaagcagaggtgtaattacagtaagctttttcattctctagaaaattaagcctgggaatttagcatatgtgtgtctgtgagcagcagagatggtatatagaaagtcaaaaaaatttagttcggagtctattcttaaaagtacaatgcaaagcaaaaatctgtagtatcattttccaaactaacccaaagggagggcaatcagaactgaaagatgcaatttcacattgagttcatacatccaaaaaatgtctagtttacatgaaaatcaaggtaacctggtccattccaattcataataatttatttcatataatacttttgaagagtatcatctttaagtaaaatggaattatcaagtatataactatgcaaacaggtcaaactgtgtccttcaagatcttagaaaaggtaaacaggaaatagaaccatgtggaaagacttgaaaagaacatacagacttttctcattctgttctatatttgttttgtttttgttaatttgtttagatgagaccaatatatgcaatggtaagccagtagatggactgactactttgcgcaatgggacattagttgcattccgaggtgagctatgtacacatttatttttcttctcatcattctgttttggcatttatgagaaccaaagccgtggaagagtgctagtttgggttgttttctggaggggaatctgattgataagcacacctcacaggagaatccgagagtaataacagaaatattggattaagaaaattagacaaagtaacaaaacaaaaccaagaccctgaactgatactactgaggttttttcctactgttaagatttaaaaagtagttcattttttttggggggggggggtagttaaaatttaaaaaataaaataaaattttaatttcataccaaatgaaatgacaccagcatgcatattcactgctttatttgaaaataacatctcaattgtgatgtgttcaagtgttacactctctgtcggagagtaacagaagaaaagttatttcatgttaattctctgctgtactctgctagtccagtcttgaaacatatttagattaaatctgaaagcaattccttttcgtacagtagaggctaaagagatgttagtcattaaattataattccttagttctaagagataaagaaattgagatctaaagagatagtgactgtaagtcacaaagccaaaaggcttttttgagaccggatctccctctgtcacccaggctggagtagagtggtgcaattacggctcaatgcaggctcgatctcctgggcccaagtgatcctcccatctcagccacctgagtagctgggactacaggtgtgcaccaccatgcctggctaatttttgtatttttttttgtatttttttttttttgtagagacagggttttgtcatggtgtccacgctggtctcaaactcctgggcccaagccatctacccgccttaacctcccaaagtgctggattacaggtgtgagccactgcacctggcccaaccaataggctctttctaaagaaactagaaaactaaagtatctcaaaggttatgctaaatttcggacttgggaaatacctgctctcagaaatgttcaaatactaccttttgtatctgactaaacttccagtcatacaatgcataagaaaactacattttttttcattttggtttcttttgtgataggtttagcattcagcttgtaggctgatgtctttccttaaattttaggtcattatttctggatgctaagtccattcagtccaccatctccagctcgcagaattactgaagtttggggtattccttcccccattgatactgtttttactaggtgcaactgtgaaggaaaaactttcttctttaaggtaacacaaatatctttgtgagagaaatttaataataatttgtaactgcattatttattacaacatatattgccttaacctccaatatgtgtccttttaaatactctgtcatcaaaatttaatcataattcaacagtttgataacagtaatggtcaaaattcaaccgtttaaccaagttaacacgttctctctatacttaaatctagaaaggagttccaaatcttttttcccaaagctgataacataattttctcttttaaattatcaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtatgtgttacataattgacaagattacaaaacttttaaagaattacactcagcattttcatttattattgactttactgtcaaaagatatctttaatggctgaaatcaaatattttcagtgaaaaaaatcaatattgagggtagtattcttattgccctcaatttttattttcatttcttcacaggcaagtcacaattcatttgagttttcaggtcactgatagaatgtcatttactttcgcttcacaaatgtgcatgtcatccttgcacaagggccatgctaatctctgtatcattccagttttagtatatgtgctgccaaactgagcacttgtcactttcaattgaaattaaatatataactatgtaatttgctgcatctattcattcaacaagtatttcagtttaataattatttttataatacccttgactaatagccattattaatatcaatatattatatgaaaaatgagaacaaaactgtttcctatacattgtaaaagttgttttccaccttttctgagggtattaaaattaattttcattttccatgtgatattctaatacataacaggtggcagcattcagcagtatatttataaacaggaacctgtacagaagtgccctggaagaaggcctgctctaaattatccagtgtatggagaaacgacacaggttaggagacgtcgctttgaacgtgctataggaccttctcaaacacacaccatcagaattcaatattcacctgccagactggcttatcaagacaaaggtaacatttttattgtgttaaaaaaatccttaaacataagtagatgtaatacagtttcttatacagtaaggcttatgaaatatggatactgatcaaacagccctaataatttaagcttactgatgaaaaactcatccacttgccttagtgaataaccaaagaccaatactttctttttccttgtgggtaagtaaagcagtttgttcaggtttgttagttataaccaatactatttatcaagtacttcttaccaagaacattatataccagtctataaccctcaatagttctacatcagagggctaaaactgagattaaaactcagatgtctggctctttccaagatagtacattgccttttaatctggtatcttttattaaacatgccacttacaggtgtccttcataatgaagttaaagtgagtatactgtggagaggacttccaaatgtggttacctcagctatatcactgcccaacatcagaaaacctgacggctatgattactatgccttttctaaaggtaaggtattaactaacagtttcccaaggaggtgatatcatttgtgaaaacatgaagataaagtaaaaatgctggctgcaatgatgactgaatgtgagaagttacactacggtacttattctaattgctgtggaaaagagttaagacttttgctttaatttcaattaaaatgatggcactgcaattcaattctgctctaaccttcatgagattacgataaaacatccagttactagagtaaacttgctactgacaatagtattttacttctatcatttgtggcatttaacagataccttgtttggcacaaggttataatgatttgcatcactgcagagtctgtaagatcagaaagaagttcctgagaaactaattttattaattaaattatattactcaggatgcttttctttgaaatgcccagaaatctaagttattctgcctgtaatttcataatatgaatgacattggcatgtattttttaaaaggaataaccccaagtaaattatttttcaaacttggttactctttaatgtttacttcataaaggagagctgaacctgattttacaaaaagatggtgaaatgtcaatcttttgaaacatcaaaaaagctgaaaagtctaggcaattgtttttctttttgttataaagttcaaattaattagtaaaaacatctctattaaaatgataaacattgtctttgagcataatagtcaacataagttattttttagtttgggcattgttttctttttaactaaaaaaatgttttctcttccatttagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaggattagaaaacttgttccatttattactattccacattctatcatgtaatccagttatgtcagaaattcacctctctgtctgttaaactgacgaccacggacaccttgtctcaatgttgtctgaagtcttactcgtctgaaaggctttggttgactactactagtatctaagaaaaacattaagataaaagaaaagcaagtgaaaaaatgagaaagcatttataatgatacaaactaattactaaatgaatgaatgaataagatgtagacttgttcatttgatttcaattctttttttcaaacaatgaggaacatttaacaaaccgaaaaagggtaaatggttaaacaacaaaaaaagcagcactttgggaggctaaggtgggaggatcacttgagctcaggagttcgagaccagcctgggcaacacagcgagaccctgtttctacaaaattataaaacaattagctgggcatggtggaacatgcctacagtcccagttactcaggaggctgaggctagaggatcacttgagcccgggtgaggttgaggctgcagtgagccatgatcaagctaacagtactccagggagaccggtgagatcctgccttaa

SEQ ID NO: 2 - protein, 1404 aa, Artificial sequence
  Mutated PRG4 protein (aminoacid change R180->W)
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 3 - DNA, 13649 bp, Artificial sequence
  Epx human gene
gcaccttggagggagtgagtagggctaggggataaggagaggggccaaatcattcatcacagatcattcattcactcaacaaacacctgctaagcacatactaaatgctgggctttgagctgggtatggggggtgtgtataacagtaaggcagattcagtttctgccctgttgcaggaggagaggcacccttccaatcatctggcccaactctcattctagggatgtgaaaactgaggcctggagagggaaagtggcttgcccaaggccatgcagccaggcggtggcgactgaggtgcaccgctcctgtcagccaacaaatatccattgagcgacacctgtgtcccaggtgctgctctgggccctgggagaagtgcatcagtgggcttggtagtagagggtagggatggagtgaagggtaggcaggaagaatgtccccaggctggtaggaggtggggtggggggtttcagtctcaaaactcccatgaaaaccagagagaagtttcagaactccacccaagaggctgggtttctagggcccagagctgccctcccccaccctagaatgggctataaaagtcccttcccagctacgtccagagaagagctggaggaagtgagaggtcggctgggggtcctcaaagtgagaggggagcagaggatcctcccgtgcaggctgtggatgtcactcacttcccagctggtgaagcctcgctgcagagatgcatctgctcccagccctggcaggggtcctggccacactcgtcctcgcccagccctgtgagggcactgacccaggtaatagtcccctagacaggcaaggaggagggaggggaaatggaaggggaagcacttgggtcttggagggggtcttgtggcttgctgaaccctgagtccccatctctttgaacagcctcccctggggcagtggagacctcggtcctgcgagactgcatagcagaggccaagttgctggtggatgctgcctacaattggacccagaagaggtggacttgggtctgggggctgcatgggcctgggaggatcagtgaggggcatgggccccagccaagttcatctcactcatcagccacctctggaccccatgaacatttcctgttggtagagcctcccttccatccatccttctgtccgcttgccctgtcctgactgtgccccaggactgggtctctgctgggtgggtctgcaccctctctccagccctcactcctcctctcctgggcagcatcaagcagcggcttcgcagcggttcagccagccccatggacctcctgtcctacttcaaacaaccggtagcagccaccaggacagttgttcgggccgcagattatatgcatgtggctttggggctgcttgaagagaagttacaaccccagcggtccggacccttcaatgtcactggtactctgatccccactgagcccgctgggcctaccctggcctggagtagaaggaatccaggagagggaggcagggtgcacaggtttggggtgctgggaggagagagggtaaagggatgggaggtacagagcaggccagctcaggtctgcccatttgccttcccacagatgtgctaacagaaccacagctgcggctgctgtcccaggccagtggctgtgctctccgggaccaggccgagcgctgcagcgacaagtaccgcaccatcactggacggtgcaacaacaagtgcgtgcggggcggcaggaggggctgcccctgcctgggggacctctcccttcctgcacccaccctctccctccatgctgagccatctccaggccctgccccctgctaacctatcccacccatggctgcaggaggagacccttgctaggggcctccaaccaggctctggctcgctggctgcccgccgagtatgaggatgggctgtcgctccccttcggctggacccccagcaggaggcgcaatggcttccttctccctcttgtgagttggggctgagggtttgggaggttgcttgatctcttaaatgcggggagtaaaacacagcccagagtcacgcaggcagggcttgaacccaggcccttccactgaccagtgcagtgacttgaggcaaataacacagctttctgaggcttagtttcctcatctgtaaaatggccctaaaacctacctcgtagagcctgtgtggatattgaagtccttggtataaatagctatagaggacatgatctattcatgctaaccattacggtgttgtacggttttttctgagaaaattagtgtcaaagtcttgctaaatataaacagctctataccttaatctggctttatctattgatctatttcacatttatcaactcatcagttttctattgatatatcgatagagcagtctatcatctacccatctatcaacatctattaattaattttcttctatccacccttccatctgctgaccccgtactccctacgtgtattatccatcacctggcaaccagcccatgcacctgtgaatccgtgctatcatctggttagctatcagtgtacaaattgatctatctatttgatctcccttcccctctgggagagctggtgaggtctgagccagtcaacctagcccctctccttctctttaccaccggaatcctcaggagcccagccagaaaccatccttctaggaatgagagcaggaggtggctacgcctccagggacaaaaggggcatggagggcagaagaggagaggctgtcaattccagcaggggagctgctgctccctgagtcctgggttggctctaataccttgtggggtcagggagcccatgtcccgtgctgatgttatttccccaccaggtccgggctgtctccaaccagattgtgcgcttccccaatgagagactgacctccgaccgtggccgagccctcatgttcatgcagtggggccagttcattgaccatgacctggacttctccccggagtccccggccagagtggccttcactgcaggcgttgactgtgagaggacctgcgcccagctgcccccctgctttcccatcaaggtacctaccctcagccaatctcccatgcccttgtgtggcctcccccaaaggcaaggtgctgggggtggggatctggaagactggagcaccatccttaaggagctgcctgtggagctagggtatgagacagagacacaagaaacacagctgagcagagaccccgcgccgtgtgtgtttgagaggtgggggtagggcaatctgccaggaggctcaggtcaggcttcatggagtgggaagcccttgacacatgccttgacccatgggtctgaatccaacaggggaagcctctgggcccctgcttttggcaacctaagggcctcttagctcttgcccttctctccttcccagagtagtccaagggtcctgcggctcttgccagcttcttgggcttgggctgtaagtagggtcctaaccttcaggctagaagccaaggacagtgtggggcactaggggaggaaagatagaatccagggagcagagtctctgctaggaacatagggttgacacacgtgcacacacacactctctcacacacacacacgtgcacacacacgctccctctcacacacacatgtacacacacagtcacccttaataggtggggccacattcatgagggatgacatgtgagggcctctggatggcctctccctgttgcaaccttatctattcctcaatccctggcttaggggcacctcctctggggagccttctcagatcttcctattacacttttaacatatgtcctcccctcccctctcctctctctccttccttccttcttccttcctctttctttctttcttctttctttcctttccttccttccttcccttccttccttcatttccttccttcctttctttctttctttttctttctttttctttcttccgtcctttctttctttatctttccttctttctttcttctttattattattattattgttttgagacagaatctcactctgttgtccagactggagtacagaggggcaatctcagctcactgcaacctctgcctcctgggttcaagcagttctcatgcctcagcctcccgagtagctgggattacaggcatgtgccaacaagcctggctaatttttgtatttttagtagagattgggtttcaccatgttgacaaggctgatctcaaacttctggcctcatgtaatccacctgcctcggcctcccaaagtgctaggattataggcgtgagccaccacacctggcccacatatggcttttctatcaagcacagcagatgccacttcaacttagtttcttaactctatgtgtgtgtgtctttttccactgaactgccagcttcctgcaacctttcctgtgtttttttaaccttcataatctccaaagcagcaagcaagaaaggggcttagaaaaggtctgtcgaattgaacagaattaaagaagctcaggcctaagagtcaagagaactaagttctaatcccacctttcctgctaactggttgtcacttcccctctctgaggctgttttccttgctgtaaaatgaggatcgagattgtttcaaaagcctcattcattcctaaccttttgagactctatgaaacaaaatgttaaggacactcagactgacggggtgagcagcatgagcctgggtgagtcaaggagggcttcctggaggaagggagttttaagcaaggttttgaggaggtggaagatgacggagaggagtgagtctgctattgagggggccccatgtcactgtctcctcttccatctcagatcccacccaatgacccccgcatcaagaaccagcgtgactgcatccctttcttccgctcggcaccctcatgcccccaaaacaagaacagagtccgcaaccagatcaacgcgctcacctcctttgtggacgccagcatggtgtatggcagtgaggtctccctctcgctgcggctccgcaaccggaccaactacctggggctgctggccatcaaccagcgctttcaagacaacggccgggccctgctgcccttcgacaacctgcacgatgacccctgtctcctcaccaaccgctcggcgcgcatcccctgcttcctggcaggtcagacagggaggaaggtggtgtcttcccaggaaacagccatccctggggtcccaactgggaagcaatggtgggatgtggtgaaggtacatggtttgggacctcagtattaggcacaccataagcatggatctgtgcacagccatcatagaatcagaacgttgagtccctttgcaagctccctgtgtgtggccagagataactgcttaaccgtctccagtgacaaggctctcaccgcctctatggcaccccagctcagccacaggcagctccgactgttagaaagtctccttattttgatcacatattcacctattccccttaatcatgcttctggaacctcagaaaagacactgtccctcttacacgcaggaatacctcaaagtagctgttaactccatctccattttttttctcttctctgggataaatttccgtagttcctccctgtccccagctgttgctagacagttaataccctcttctggaagcataatttgaactttcctcagggtcttttgtcccatgtacatggttacaataataatggtagtcatttctcattaagatatttatcttattattattattattttaagatggagtctcactctgtcgcccaggctggagtgcagtggcgtgatctcagctcactgccacctctgcctcctgggttcaagtgattcccctgcctcagcctcccgagtagctggaattacaggtgcctgccaccacgtttggctaatttttgtatatttagtggagatagagtttcaccatgttggccaggctggtctcgaactcctgacctcaggtgattcacctgccttggcctcccaaagtgttgggattacaggtgtgagccaccacgcccaaccttatcaagatatttaaatgtgccatctctaaaccttgcaacagccctgaaaggagggaattattattgttctcattttacgataagaaactgagactcagggaaagtgaatcatgcgcccaagagcacagagttagtttcaaatgacagagtccacttgagaacacagtcagtgtagtatcaaggtctctgcccagtccactgtattccactgttttcctcacagagagtcggccccaacccctcttttgaaggattgtacatttgtgtgtcctggaaagagcagcttgtgccttcgggagaggtcaactgaactaagaccttgtgcaaaagggtttagttaaaaccaagcaaatgatggaattaactttctttccctgcctgtagtaggtctgttcgtaaggacagtctctgcccaaccctccctctcttcctctttccctctggttcacgggggagagcagtattataccaagcccaaagataggaagaggtacaccagcccttggcagagaacacgcaggcagatgcagctcatggacaggtgggctctcagtcccaaagtcaaggtgcccttgcccatagctggtccaggctatggatctggttgtcttgtagagtagaatccttaggcaaagatgcccctgcctgagaccagcagctgccctgtgctttcccacctcacccaagcctgctaggaacccccggtccttccgctcctggcctgtggtgttagggaggagggtggcgatgaggagcctcagcacagctgaagagatggaggtccagtgagggccaggagtttggcccaccccgtctctcccatccccagccctgggtctaccctggtagaaagacatttctctgggaaaggctgcagtaaatctgagcttggggttttcaaggtgacacccgatcaacggaaacccccaaactggcagccatgcacaccctctttatgcgagagcacaaccggctggccaccgagctgagacgcctgaatccccggtggaatggagacaaactgtacaatgaggctcggaagatcatgggggccatggtccaggtaaggagctctgcatcccagcatcccccagatgacaagcttggcatgagaagcagtccttaacacatccttgcggatgtgcctaaaaccagctgggtctgggcaactggcggagcacctggacctgtcctctggccccgattctgcctctaactcccgtgtgaccctgtctgtgtcactcaccctctctgggctttgtatctccacccaccaatagtaaattaatgttgtcacatttgacgtgatgacaataaagaatatgtctgagccaccctttgaaaaggcaagggtatgggtgagtagcctctggggaatgttcctcctgtcttcccttccagatcatcacctaccgagactttctgcccctggttctgggcaaggcccgggccaggagaaccctggggcactacagggggtactgctccaatgtggacccacgggtggccaatgtcttcaccctggccttccgctttggccacacaatgctccagcccttcatgttccgcttggacagtcagtaccgggcctccgcacccaactcgcatgtcccacttagctctgccttctttgccagctggcggatcgtgtatgaaggtgaccaggttttccagggggcaaatgggggtgagggtggggagcatgccctcccctaggtgggccaagcttactgccaggaagccaggctgctgcagaggccactgctaatatctccccaggacagtggaaacaaggcaggtgccagcaagaccctcagtcacgggcctcccatcctgtgtggaatgagaggattttttaaaggggtggagactaatgtcagattgatggggagctcaccttccattccttaagaagtacctcccagctccagctgcttcatgtctctccagaactctgtttcctgacaaacgttactaacatacccgactggcttgtccagctctgggctagcttggcatcatgtgataacccaagtagcttcccagaggctggtccaatctgtgctgctcacattccctgccaccagggggcatcgaccccatcctccggggcctcatggccacccctgccaagctgaaccgtcaggatgccatgttagtggatgagctccgggaccggctgtttcggcaagtgaggaggattgggctggacctggcagctctcaacatgcaacgaagccgggaccacggccttccaggtgagggggctgtccacctcttctcccagctttgctcgggccaggctgctcaaggggttctgggaagaccctggtacctcctttctgactgggactgtctacaggatgtgcaggagtgcagaggcatgcaaggccaaggtcgatgtcccaaagcactcctggaacaccgcttgctgtcctgccatggctcccccatccactgtaggggctccagctttaggttcaccacaggaccagcacttaggctcaggagctgaaaaagggaggctcgaactttccaagtagaaaaaaagcaataggcttaaaaggcagggaagaactgctccattcccaacacgctggcaagttccaaggaggaaggctaaaattccagagctacagaaacgctccttgctctaagtggagaaaggcaaagagccttagaggcagggaataggtggaataaaactaaggctgcagtggctgtttggtgagggcacaaatgactttatcccaggaaagaagaaaatataggcactttgttagtcactttcatgtatattaatttattttgttctcccaatcaccttgagaagcaaatgattacagccattttatagatgagaaactatgttcagagacagctagtaatttgcccaagatcacataaataagaagtagtaaaatctgaatttaaatttacaatttctcctttcttccccctgttcaaccccagtaggggaaggttttggccactgccttgctcctctctccttcccacctcctacctccctgtctagaaagaacctgaaggcccatctgcccatgtatgaagcagtggtggcacctcctactgcccaccccacttcacacagcgcctggtagagcacagcaggcactccggaaacctgagcttctttcctttcttaaacttgggcgctcctggcagctaaggagctgtgggcaccccctgccggagagctgagatcgctagccacccaagtttctattaggttttgaagggaaggcaagggttaaggaaagacacaaagagagagggcggctgaacagccaatgcaggtatttatttccagcataaaacctacagaggtgggggaccagcttattgccagaacctgccgccgcctacagctggggtactgggtatgggtatgggtatgggcaggaggggtctgggtcgtatggcttgctacccagcaggatattgataagatgtacctatgatcaggtccttttttcagtgggatgtggtaagatgttccttggacctttgcccagcaggatatgatagggatgttccttcagttgggcctttgcccattagggtatgttatgtttttcatggcccgacctcctgtggaacgtttcactttgaccaaggtctgggaaatggtggggggcttagaaaatggtgcagtttggactaacattcttgccttctactttggtataaaaggaagagctggagagctgagatcgctagccacccaagtttccacattgcgagcttctgctccccgcaatacctgtgccctccagaaatattatcctgacactagctcttgcaatctcctctaatcccttcccaagtccaacatcaaactgggaactggacaacctggtttgagctccacttaacagtaaagtgaccctgaagcctatggttgctcaactttagtaaacataccagatatagttagcatgccataaaagcacctcaaaaaggtgctgtgagggtcctgagatattcacccaatagtgctcagtaatggtaaaatactgtgtggttttcatgggttccgctgggggatggtgtgtgtgagctgtagttcaatggatgacgttgggctggtgtcccttcgggacatggcaaagctatctcctaccttgattgtcacatcttatgatgtggcttggccctgcgtgggatcctgatggctgacaaattggtatggcttgtctttggaagtttctgaaaactttttaaaaacttctttattgagatataccataaaattcacccatatgaaatagacaattcaatgattttagtgtatttagagttatgcacccatcaccatgatctaatatcacaccattttcactccccaaagaaaccttgtaagaacagtcaatccccatccccaccccagccccaatcacctctaatatactttgtgtctctatattgacctatgcaggacctctcatatcaacagaatcatagtgtgccgtccttttctgagtagatgaattttatcattgctctccatttgtttgcttgttcattcatttattcatttgctcactcattcattctacaaacctgatggagagtcttttatattctgggtactgtcttacttgtgtctaatgaatgcagtcgagcccagtaatatcactaagtgtcggatttgtggttatgagttttgggttaaaacagctttttaggttaggacacactcctcagtctgatgcacactgagcacagttgtccactcacattttcaatcagaggacagaaacctagtctttggaagctctagggactcttctgccagcaggatcttctggttctgcccaatattgactggccacagcttccccccagaggactggtggagaaaaacagaagctaatgggagatcagcaagactgaagctgcttctccccgttcccctgcagggtacaatgcttggaggcgcttctgtgggctctcccagccccggaatttggcacagcttagccgggtgctgaaaaaccaggacttggcaaggaagttcctgaatttgtatggaacacctgacaacattgacatctggattggggccatcgctgagcctcttttgccgggggctcgagtggggcctcttctggcttgtctgttcgagaaccagttcagaagagcccgagacggagacaggtaagtgaccctatcataaaagacatcagcaccagaggcagagcagaaaaacactagcatttcaagactaaacattgaagaacactgctctttttagtatcatttcttccaagttcacaggatctgaaatcaggaggctcctctctgaaaagctgggtcaaaggaagagagacactgacccaggcaaggcccatattgcctgagctggagtcatcttaaacccagaggttgcctggcagcctcactattctgggttggtggatcccagaaaacatgggcagaaagggctaaatctggtttccctccaatacatttgtgatttaagacctgacggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccggctacttgggaggctgaggcaggagaatggcgtgaacccgggaggcggagcttgcagtgagccgagatcctgccactgcactccagcctgggcgacagagcgagactccgtctcaaaaaaaaaaaaaaaaaaaaaaaaaaaaaaaaaaaaaaagacctgtctccttatttggagttggtattgcccgagttcaaatccagccctggccacttaaattactgtgcacttaatactgggcaatttatttggccactgaatttgtttcatttctgagattccaatcttgcaggaattttgtgagaattgaatggaataatatatgtaaagtacctggcacacaacaggtgctcattataaggtaattcctccccagccttcacccacatctctcgactgcctggtaggttctggtggcagaaacgaggtgttttcaccaaaagacagcgcaaggccctgagcagaatttccttgtctcgaattatatgtgacaataccggtatcaccacggtttcaagggacatcttcagagccaacatctaccctcggggctttgtgaactgcagccgtatccccaggttgaacctatcagcctggcgagggacatgaggcttctgcaggtaaggggaggccacctccagcaccctgggctgggttaagccctcacatccttccctggatggatggctgagtcctcttaggtctctaagcagagaaaacagaacttgtcactaggtactctttccaagtggcttcccaatgtgctagtttctgggctgacagtcaattccaggccctaggactttggggggaaattaggagcatccaactatcctcagcttggtcactaatacctgaaaacagctccatttctaaacatccagtaaattcctgacttatccagttctgttacttccacaggagtctatcccaagtctccaacttttggagacaaggggaaggggaggaccatgaggctgccttgtctccctggagcaagtgcaggctgctgacgcttctgctggctacagctcagagctgggttccccagccaggagtgaaggctgggggctcctatcagcaatggaccttcccgccttgggagcctcttaggtattaggctatgaatcagcgccacgtgcaaaggcttgggagccaagccatgtggtcttgcaccccaggcaagaaaagtcagctggagggtttacagcactttctactgtttcccagccctccctcccctccctcaccatgactaagagaccactcggtcctagcctccagacaccccacaatactcctctgagcctgaggccaggcagcatgctctgcttctaccaataaagcactgctaagggcaggggctgctgtttctgtgttgggctagggaagacgtggcctcagttccatgctttgctccagatgtttaagagaagtgctgttaacacgcaagctggcacaaccatctaagcctctcctgggggctgaattaggagagcccaaagaaacactgtggagtagggaaaggcagaaaaggggaagggcagacatggtaggggccccgacattaagaacaccctagccataccagcaacctgagcagacaaacagccaacagaacatcagtcaaaataaaaggggtttatgtaacaaaaaacaaaaaaacaaacaaacaaaaaaacacagtaaaagataccacccagctagcagaaaggactcagcactgccttcagccttcacttactcagaaataactcatatctcaacctcagcaagccccaagcagtagaatgaggcttccctggaaagaggctgagactcacaggctggggatttaagaccctctcaccgtccaccttccttccttctttggtcttggaagatgaaaggctccatttttaaagggtggagaacagcagatcccctgctactgtgagacaagccagaaaagtgagtcaag

SEQ ID NO: 4 - protein, 715 aa, Artificial sequence
  Mutated EPX protein (amino acid change Q122->H)
MHLLPALAGVLATLVLAQPCEGTDPASPGAVETSVLRDCIAEAKLLVDAAYNWTQKSIKQRLRSGSASPMDLLSYFKQPVAATRTVVRAADYMHVALGLLEEKLQPQRSGPFNVTDVLTEPHLRLLSQASGCALRDQAERCSDKYRTITGRCNNKRRPLLGASNQALARWLPAEYEDGLSLPFGWTPSRRRNGFLLPLVRAVSNQIVRFPNERLTSDRGRALMFMQWGQFIDHDLDFSPESPARVAFTAGVDCERTCAQLPPCFPIKIPPNDPRIKNQRDCIPFFRSAPSCPQNKNRVRNQINALTSFVDASMVYGSEVSLSLRLRNRTNYLGLLAINQRFQDNGRALLPFDNLHDDPCLLTNRSARIPCFLAGDTRSTETPKLAAMHTLFMREHNRLATELRRLNPRWNGDKLYNEARKIMGAMVQIITYRDFLPLVLGKARARRTLGHYRGYCSNVDPRVANVFTLAFRFGHTMLQPFMFRLDSQYRASAPNSHVPLSSAFFASWRIVYEGGIDPILRGLMATPAKLNRQDAMLVDELRDRLFRQVRRIGLDLAALNMQRSRDHGLPGYNAWRRFCGLSQPRNLAQLSRVLKNQDLARKFLNLYGTPDNIDIWIGAIAEPLLPGARVGPLLACLFENQFRRARDGDRFWWQKRGVFTKRQRKALSRISLSRIICDNTGITTVSRDIFRANIYPRGFVNCSRIPRLNLSAWRGT

SEQ ID NO: 5 - DNA, 28057 bp, Artificial sequence
  PON1 human gene
gaagacgttttgtcctgttctgtagcatctagaacagcgcctggcacatagtaggtactcaataaatgccagctgcatgaggaaatgaatgagctgtgtgggggatgtacttgagtgaactctaaagtcagagtggtgttgagagaaaaatgcttgaaatccagatgttggaaggtgacacagagtagtagcctggtgagaacagttagatcttaggggttcctactacagccctcccttccgcacctttttggctgtcaccatgatcaagctactgaatctctctgagacgcaaggaccgggatggcacaaagtgagtgctcaccaaagcttgactgtcctttcccatggcaatttacttcagcttgtttgatttcccctccccgactggactaggcacctattctctgtcttctctctttacagttggaaggagcaaaatgggacttttggctgaaagtgctgagctcctgcggtgggggctgaccgcaagccacgccttctgtgcacctggtcggcccagctagctgccgacccggcggggaggggcggggcgggccaatcggcgctgccccagcagggctgcggctgcaggcaggcagagcctcctagcccgtcggtgtctgcgcccatcgatccctttgtctatccccgaccatggcgaagctgattgcgctcaccctcttggggatgggactggcactcttcaggaaccaccagtcttcttaccagtaagttgggttgtggtgagggctgcagccttcgtcctcctcactccctgccagctccacgagcccccagaaagttgggtccaggctgttaacagaacatttacaagcaggtgctagatgcacactctgcgggggtttcataaatgtttgtccagccgtcatgtgtcacttgaaaacaaactcttgctcatggcctgagacatgggataaggaggcaattgccaggggtgtgagtgcccaggttgtcagcatacacgaattaatttttcatgaagatttgtgtgccctggctacagattcacttctcaacctggaaagaaacaaactaacctggctgggggttctcctgatattttgtgtattagagaaaggagcccctgtcgcttctgcgccttgactgcctggccatggcaaaccttccaggcttgaagattctcaaattgaggctactgagggccacagcaaaactgcgtgtggacccgagcaggtggagctaacgtgtgctgtgatggttcgcttcagtgggggcaggcggggaagagggaaccctggagccagcatctgatgtcctggggtttagctctgccccatgagtatgcgatttgggcaaatcactttacatctctaaccttagctgcttcagtaaactggaaatagtaatacacactgcctgcgacagagtagctattcaataaatgttagttccctgcttccctttaaagtatttaataaagggttaagtgctatacaaggggggtggggatattttaagaatgtatcaaaagtttcataacttttaacaggagttcaaagtctacagatcatcctagtctctaaggcttagtcaacaatgccttatttactttaaagtaaaacctaataaaaattaaatataaaaaaattatatgagcttatggactaattagaagcttacattcagaatgggtttttactatttattgtggatgtttttctaatttttttaagttacagagtgcagaaaaaaagtaagaagcagttggtattcttggcttttaaatcatgtaaaaattacagtttgtgagtttttattaatcacagtgctggtggctgccaaaggagattataggtatagataaaaatatatagataaaggatgggttgctgagcctcctggagtgacccagacagaggaaggaagggaaattttacccactgttggaaacccctaaaacctagctactcaactgggtctggggaccagatgcctgagcatcacctaggggctccctgtacaggcagaatctcaagccctaccccacactattgaatcagaaccagcgtcatggaacaaactccctgggggattagagatcatgaatctgccccagggagagaaggcaagagatgatatcatgaggcctgcatcaggagcaagccaaatccccaggatagatgcatccttttttcttcagacttggagtgtatgtgtgtgcggctcacaggaagctatatatgcctgggctggagggaacttaaggaagcacacaagggatgcctcaagagatttctgagtttcccaggtgggagcagtacagagaaaagaactgagagagttcccctagaccacgagtctcccacccacgagtctcccacctttgtagtcggctgtatgtgccgcacaagtccagggaatgccttggatgatggtagaaacagctcccctcccctaccccaacttgggctatggaatgttgcagcatctgtatggcaacttccttgtccctacattgctaattgcaatacgtgagtccactgagtttcccttctcagaaggaaaccacaaagctaacgttgggcctctcgatgcatttctgccattccatgcttactgtaggcaacacacagttgtgggaagagagtagttgtggattcctgatgacctgggttagatatcatttctgcaactttcaagttattttaccactttattccagaaaagttccagccagtttctccacccgaaaattaggggataataatacatattacagcatatatatatatatatacacacacacacacacacacatacatatatttatatttatataaaacatataaataaaactaagtatatatatatatataaaaatcaggcatatagcagatgtcctgtaaacgttagcttttatagctgtcatatgtgatctcaaaaatatagagtgatacttgaatatgtattataatcatacttcattgaaagtctgagataaggacaaataatcctccaaccctccagtctcccctatgaatgcaaccaacaccatcttctcagtctgatgtacttgcctagtatctccacaaaacacttcagtggacatctgcagagtaccttctctttgcaaacgttgtgtttctgctggaggttataggaatgaataggttgcagtattcactcaccaattttaaagtctactgggagcaaaagaacaaaaactgacagctgtgaaactgacaactatgacaactatggtaagaaagttgacagatgtatatgtgaaggttcctgagggtaaactaaggagttattaatcccaatgcagaagactggagaagctacaaagcatcatgagtaggggtctttccagtgggtaatgggctaaagagcattctaggtgaagggaaaagtgtgagcaaataaaaagaggtatgaaagaaatacagggaagagtccttagtatcatgacacttgatttcaaatcccaactctaccctcttaaataagcaaccacatgcaatttactcagcctctcagtgcctccatttccttacctacaaaacagggtaaatattcatctttttctcaaaggtggctaaaaagattaaatgaggaagtatgtgtgagagcccttattggagcttggcacatagtaggccatgaaaagcaatttgaatgaatgaggcgtgtgtagtagcagaaaatagactgattagagtgaatttggccccaagaagagtttgagaatcgatacggatttcttaatgcctaggcaaagatctctgcttttttctgcgagcagccagcatgtattttcaccgaatacttctgccttggtttggatgttcgtcctctccaaacctcgtatttgatctctaatgttggaggtggggcccaatgggaggtgtttgggtcatgaagatggatccctcatgaatagattaatgacctccttgggagtgagtgaattctcactctattcatttcgagagagctggttgattgaaaagagcctggtacctctcccctctcttacattctctctcatcccatgatctctgtgagtactggcttctcttcatcttctgcaatgagtggaagcagccagagcccctcaccagatgcagatacccaatcctgaactttctaaccatccagaatcataagccaaataaaccttttttctttatcagttgcttagcctcagatgttcctttagagtgacacaaagcagactcagacaagttcacagcaaaggctgtatggtttctgcgcagagttcagagtctagtgaatcagagatatatctcagacattgacccttatctcttaggcccagaacatttggacttaagcctttgacaagatatttcctaataaattgaaaatgaggtagaaaaaatatgagttggatgataataacacaggtgggtcaattttcaggtgcatcttccagcttttaagcagtgttctctgtagattttgcctggtcagagagctaaatgtgagctttgtgttcatacttgaattccaatccctgctcagccactttataactctgtggccttagaaagccacttaatcttggtcaactataacttagggaaaataacacctcacggaggtgtttgaggattaaacgcgggcatatacggtacctgaactatagtagtaatgattatcctgttgagatatgacatgtttgggccacaaattggaacccaaatcaacaagtgctgacttaatgcccccttgcccaccctgggctccctttaactgtgattattctatgttttccaggctagagatcatcttctatgagagagaagcctgaattagaataggagctgaccagttctgcctttctccatcttttgtgaacccatatgtttagtgacccttgtgagtcaggcagcatgagtattcttcccattggttttgagtctaagaaaagtgaatttgactatccttggtgtttttcacaagtcttggcccattcagtgattttatgtccttgaatctctccttccatgccagtctttgtgttgtcctcggttatgaccatcacccttaacatcttgtacctgccctttggttccttgagggcatgctacaaatattcactacactttccctagaccagagttttttcaaatggtgggtaacaaagtcaatttagtaaataattaccaacatttaaaaataaataaaacagaattgaacagaaaagttatgaatgtatgacacagattaggatgagtattgttttgtgaagcttgattcagatgtgtatgtgtatgtgtgtgcatgtgctgggtgttcgcaccatataaaatacttttcctactgtgagtctctgtctgaaaagtttgtaagccactgctttatcacttccccttttatttccttattgtcaatatttgcaattataatgtcagattttcattttgaaaatacctttccctttcttaggttatgggacagattccaactttcctttgaagcctgctttctaagttcacatctatcaatcaggaaaccacagtctcctcacagtccaagccttcatcatctattcattcatctctgtatttctagtacctagactggtggctctttactctggttatacgttgggacaaactgtggcacttttacaaagcagattattaagaggcactccttgcctcttacgtcagatttttttagacagcgaaaatgatagccagtggcctgatggtaggaatgtactcatttttttctgaatgaataaatgaaagatcaatcacccccaaattcctatcattttgtaccacgtcggtaagaattttatctagcgtgtctgttccgcctgtagcccatccgcctcctgtgagttcaagtcatcaggaggacaagtaaatttcttagcctatctgaagccaaataagatttctagggatatttggataattgtcactacttgctatgacaatactgtatcactagttctctaaaaaaaaaaaaaaaagtttattgtttaaatctcttggttgtgctaacgtcgtctgtttctttcaaatgcagcatcagtagcttcgctctgctcaggcagccggctggcacttccttctcacatctgttcatttttcctttgtcctttactcagacattttctatggtgtttatgcactcatatctaataagtcgctcctgtgtatgtgtgtgagtgcacctgtgtgtgtgaaatgcctttttacatgcaggccttgtgtatgattttctgaagttacaaatcctttcaacgtacctgtatttttcaagcactacatcacaaagcagatgcccagtccaggctgcggagtcagattgggcagatgagtaggaggctgcccatctgtcagcaactctaaaacatcaatggaataaactggaaccatgttgccatttacccggactttccacctaggagtccttacacactgatgaaattgctcctgttcctgccaaagtacacagagccctagagaggatattatgtatctcttatgtgtacctatccacctctgctaaataaaaactaaatattgtacccagagttccccacttgtcttgccctggccttgcctggaaaaattgcaggaaaaactctggagttgaaactcaggcaaagtacagtgaaggagaacttttgtggactgcctacctttgcaacttattataatttctgaatagttttgtgtgtgttttatttctttctcccccacagaacacgacttaatgctctccgagaggtacaacccgtagaacttcctaactgtaatttagttaaaggaatcggtatgtatgtgagaggaagaagggtctcatttgaacttccggtatttttttgctctttgtgtgattaatgtgcctgttcaattctgtccaaaaataaaattttgaatcaggagagtcagtggtagttaatgataaataccatgttgtttgacttttgacttcaccaacatactgccaaggtgttgggagttttggttgtttgtttttgtttgatcaatagagcaaaaaaaaaaaaaagaaaaaaagaaaaaagaaaaatagaataatgtttgattccatggcaatctaacctgaggagatggcagaagaggggagggagagccagataaagcattgaataaaggtaacttttttttagaaaggctctgacaggactttgcaattaccacaaggcttctccttatgcatctctcttttgatattggacaggaaaattttttccagaagctctcagcagatttccccttagatcttatttgtcagagctggaggacaggctggtgccctagttgcaaaggatgctgggaaagtaattatcaagcctccaatgtgggaggcaggccctgtcagcaaagataaaacggtaggagaatagcagctgggaggcaaccaacaaggagagtccccgggctcaactctccagctggttccaaccaaccagctttgttatttgaagacaattggtttgagatcatgcttttgtgtgctttgggtgtttctttttctttcttttttttttttttttttttgttcttttagaattattcatctcagttgattaaggctcaggaggctagatcagatgacttagtaccactaaatttcactagatatcttatggtgcgattgttttctagaaaaatatgatattgatatcaaatatcactgggtttttgaaaagcaaactttccactaagtgtagaaatacctatcaaatagttcatttttctctagataaattgaaatgttaaaaatttacatgtacttaaagtataactaacttgaagacagtaatatttaaccgatgtttataattagtagtaaaaattcatattcttgatcacctaaaatgtatccccattttaactcaactatttggaatttgcttatatcttcaatatggctttccaagggtttaattgatttggaagtattaagtatcaacatatatttgtaatttaccttgtatctttagaaaacaagttcttttcaaataatcatggcttttgtacgttttgtgaatttccatgaactttaagaggattcagtctttgaggaaaaagctctagtccatcaatttaaaacaaatatactaagtgtaaattttaattaaacttgttaatgaattaatatatttttaatttccatataatcgcattcatcaatttgaataaatgaaagaatgaattattctgaacctattaaagaagagtgatgtatagccccagtttcaagtgaggtgtgataaagaaatggatccacatcctgcaataatatgaaacaacctgtactttctgttctcttttctggcagaaactggctctgaagacttggagatactgcctaatggactggctttcattagctctgtgagtgttttctttcacttttctgtgttctagaacgttttctaggactggcagtttaagtctttcacttaggctttctgtatacccatgcccactttcaaataaaatgaacagtcatggtttaaaagacaaattgtgtatgcatttacacttgtgctataattatgatcatgcgtgttctatgcatatttgtgttcttcggtctacattgaaccatattacgttagcttctattgcagcaatttttttgtgagttcagtgtattcaagaattgttttcctctcaattgtctattccctaagcacttaaaattgccatattcaaatatttgctaaattaaatatacatttaatgtatactacttcacaggtcatttcaactttcacatttttaaacaaatttcagaataagccctttaatttaaaattgttgataggtcccaattcccaattcccaagctttgaagacagaatccaccatatttgctgaaactgtcctgcatatagatcgctatggtagctgatatagtgcaaaacaaaagaagtccaccatctggcctagggatcataacataacagagttgataggaaacgacagacaatcatccagccatttaaaaacctttgctaccccttgtaatttacttagtccagttttctcttacaaagatgatagactagtcatcctgcaaagcaatgtcttgcaaactttcttgagcaacataaggtgtatttcaagcaaaatcttatatgggaacacaatatataaaacaacagctttattgttttgattgaaggctaagaaaggagtagttcctctggcccaccaaaactctgtctttgagatgcttccaaggacctaccagggaacagtttaaaagccataagcaatattctctctgtattccttgcttatatcattgaagactattattaattgcctttttggtctgctcttccatttttgatctttctcatgggctggaagctgtagtgtcgtgcacatatgactttgctgcaggggatgaaagggtttgtgtggggtggcctagtggagcatgtggcttccagaagacttctcctggggacccattacagaagctaactaagaggcttcctatgtggccatctggctagccctgtggtttggagcagtcaacatgaacccatgggccatgcagcgcctctattagcatttgaagtactggtattgtgcttgatttttctcctccatggtttataagggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaaggtaacaatatttactatttattaaaaccaaaaccaaacttaaaacaaaaacagaaaaacatgccaataaatgaattattttttctcattcaaacaaagcatagtcatgaataatgttcttactctgggttttaaacttaccctctatgttaattgaactttatagtaaaaccacttaaatacttgagaatgctaaaatatttaagatttgtaaagcatctaattttgtaaaatactaacattcacattatccctataattaccataataataatatccagccctgatcattatagcttttccattttaaacgattttcaagactcgggcatttacaggaacagatttttctaacccaaggcatctgtaagctataggtccagtgacagaaagtcaccatggtcagctttctgccggtcccattatcttcaatgcctcctgtccgctggttcctctgttctgtgttcaaaatgctcctctgcatgttggtccctttcaacataacttggtgtcatcctttatgtctttactccctttctccctttccatttgctgtcacttccttgtctctgtattaattttcagccccagttgcacaatggtagcacctgaagagtttaaaaagactcgatgcccagcccccagcttgacagattccaattccgttagtctgagggaggcctggatcacagtagttgttaaaagtttttcagttgataaaaatgtcaggctagggttaagaaccacgtttatacccattacctccatttcatcatcagacgatccctcattcagttcctcttctggactttccccactgtgggtggctaaaattgctaccttgagaatcatcagtgatgttcctactaacaaatctcctctcttcccttcctccctctccttccttccctcttcccttccttccttccttcacctccttttgttttttgttttgttttgtttttgagatggagtcttgctctctcccaggctggagtgcagtggcgcaatcttggctcactgaaaacttcacctcccgggttcaagcaattctcctgcctcagcctcctgagtagcggggaccacaggtgcatgccgccatgcctggctgatttttcgtattttagtagagacgggggtttcaccatgttgcccaggctggtctcgaactcctgagctcaggcaatcctcccatctcggcctcccaaagtgctgggattacagttgtgagccaccgcgcccagcctacttcacctgcttttatgctgtttaccattttctcattcacaaaacttgctcttccctgatactgcagtgcacctagctgttccagttctcatcctactacaggttttctccccaattcattgttttgtcactcattgctacttccctgagcactaccccagaggtttgatcttttacttcctctatattttctctattttagaactttcaaaccagtgtcacactgcctgctttctaggaggacatccccaaataggggtattattattcttcttcttttcattagcccttcacttttaatgccgcaggtgcttactggacacttctatttggattcctttcctatagacactttaaactcaaaatgttgaaagtgattttccatccttttatctttccttctcttccccatgtgagacacacacacacacacacacacacacacacacacacacatatacatatatatccctttcaatggactttccactcatccacccattcatcaccattatgttttattttaaaaatatttttaagtacaaagaaatatagtgtttgatgtgtggatactttttaaaataaaaatgaggtaatattacacaaccccttctataaatttcttctgcattaaacactacatcatggactcctttatatatagcacatttgcagttgctttgccaaggcagttcgggattcagttgtgtgggagcactgtaatatagctcatctgctaacaaatatttaggttgttcccacctttatgctcattgcaaacaatgccaaaataaactttctcgcacatgtattttctgagactatatccttaggatacatttctagaagtagaatccactgcacaaaatgtgaattcaggttttaatcctcacttgaatttgatttccaacaccctccttcctcagctgtattcagggcctttggattcttcctgtcaaatgcctctcatgacctttcctgccttttattgccagtggcatcctcttggtccagtcctttctctccacattgctggattactgctgtagcctcagctcatgttactccttggttcaaataccccagtgagcaaagcttggatcttcatcttaagccctaacaagactgattctggctccctgtggtttcagccctctcactctcccactctctcttgccttcactctgctcccagcacaacctcacaattaagtgaatatgtccaggaagttctgccccggggcctttgcacactcccagcacaacctcactattaagtgaatatgtcccggaagttctgccccggggcctttgcacactcccagcacaacctcactattaagtgaatatgtccaggaagttctgccccagggcctttgcacactttctcccctcaacctggaatgctctttccctagatatccacattattcgttctcttatttcacttaggtttttgctctaacatcccctcatcagagagaacatgcctgactcccatgaagataatacttcgttactctctattcttttatcctgttgtatttttcctttgtagcatttaacgccaactaaaataaattaaacacttgtttgctgaatggggtaatgaaaatcattttagcagctttctctgttcaaatcttctcaattcaacctgccaaattttgcatcttaacatggtgaaaccccatctctactaaaaacacaaaaattagccaggcatggtggtgggcacctgtaatcccagctacttgggaggctgaggcaggagaatcgcttgaacctgggaggcggaggttgcagtgagccgagatggcgccattgcactccagcctgggcaacaggagtgaaactccgtctcaaaaacaaaacaaaacaaaacaaaaacgccaatttgatcatgtcagaggtttcaaaagacctactctttcttaaggcaaagaccataatctaacttttacttgccagctctgatcacacattcttgaccgcttagactggtcacctttgtgttcttaaagtcccacttatccaaggaagctgcttctgtttgtttgctactatttctcagatgttcttctatggaatgtatcatctcacactgtgtaaattacacctgatcacaccttctcatattgggagttcccatggtttttatccctttcatcgcaactgtgcatttcgtggagaccatttgaactggaaaacttaaaggattctttggagttttagtgtcagtgatactcattcttgagaatttttttttctcctgtggagttttgagttttcagagactgtcactggttcttccttgagccacttttgtttggaacaggagcaatcaccaatgcttctctctacaaagagatgaaaatattccatctgcctgacatcctaactactagctggaggtgggttgaaattggtttttggagagagggctgacagtgagagcttagttaatgtttcatttttgtttttaatctcctctcagataatgccatgtacctcctggtggtgaaccatccagatgccaagtccacagtggagttgtttaaatttcaagaagaagaaaaatcgcttttgcatctaaaaaccatcagacataaacttctgcctaagtactgagatatatcacatttctattttcctttagctgtagcggatagttaatccacttttagtttttccttttggccagagtaggtatggctgttcaactctcatttctaatgatcacaaactcctccacatgctcttctgaagaaccttctgaggactcaagcccctcagagacggcccattcttttcctctcttgtttctcgagctgattggatgctggctcctggcctcaggatctccattcaagttattaccaggaaaaaaatcctcttcttcagaaccagacttagtatttcaaacccaaaccggcttcatagataaatttgacatcatgagcttatttatttatattaaaaagtagactcttagatgtttccatgaattagaaacacagacccaagaggagaccctgaatcaagtgcatctaagacctccttatactgcctccgctgctgtttgattgcacaaaatttatgcaatagcagatctgtggtaacactcaattccccctaggcaccttgttcatgtaacatatggtgaaataaaacactcaagaatctccaatctcagtcaacttcctcacatgccctgtgaagatttggcttgtaaggccagtgatggatttaggagaacaatccgactctatcttctgaaacatttacctccaactgattacgattttctagaatcccaacctatttttatataatttatgtacagcatttgactacctccatatctgtaaatctagatttgtactctttgcaggtacctagctaagggcctggtatgtagtgtgcactctgtgttaccaggaccccttcctcttctttcttatctggtggtttgaggatgaggaaaatgagattggagagaaattgagttactggatgaaggccaatcaactcactagtggccaagtaggaaccccagctcaggtcatcttctgccccatcaagatgaataaaagttcagagaacagcttttggtggaagagtttattcgtgcatttatttaacaaatatattttggagagagaataaggtcactgagtggagaatgtgaggaacaccaagatttaaagtagagaacagtatgtagaatggagaagctgggcaaatgataacactaataaatgagctttctctgtaagtcaggtgctacactaactaattcaaacagattattttattgatgttttacaaataccttctgaattaggtgttattattatatccattttggatttttaaaaagttgaggcctggactaattgttaagtttccccagatgacatcatcagtaaatggtgcagctgtgattctaatcctggtaacatgagttcagagctggtgctttaaacctctacaaaatagtgaaaaaaattatgtgttttaacatttaatcttggaaatctcagattggtctacgttaccggattggagtatttttttcttgagccagttgcaaaaatattggtcttaggcagcattggagactgaaaataaccacggtgccgtaattaactagagttctgctctctcaatccaaggattagctgtgtgtattttcttctttgtattttatggttttttataacattaatattccaataactaaatctattataatttaaacttgttattattcatatatcacaactgttattattggtcatttactgtatgccaggcatggtgtgaaggcctttgcattcattatttcattgtattcttactctagcctttcaaggtatacagagaaaacagaggttcagaatagtggctcaaaatttagtgacaatgctgtggccccacattataaggatagcaagcagtgtcaggatgcaaatgaagtccatcagatttcaaagctattactctattaccctttaaaaatttgttatttatttacttattttactacacctgggggtagtaaattttcagcaataaaaagctattgctctaaaccattccactctgcccctttctgtgaaaaacagacaaatgatagtccagcatactaatttaagaaatttacttttaaaaaagagagagtaaaatcagattgtagatttatgtctggagcccatccagactaatcctaaattccgagacctttttttttttttcatgttgtaatagtggctagcattgattgagggtttattgaatgtcatgctccctgcaaagcacacaacaggcattgtctcattgtaattcccactgcaactccacaaattaagatcctgacccttagttttctactaatgagctgtatgattttgagaatatcatttaacctctctttacagatgagaaaactgaggctctgcatgcctgtctaacttgcctgagagctcagagctgcgtcagagctgatgcgctggccatgacgcctctgcaatagagcagagataattttctcccagatcatgccctggggaacttccatcacctcaatcagcaaagaatgactcacttatgtgttccctattgtatccattaaagcagaaaaatagactcatatatcttacattttcagtaacagagaatcctaatcagaaaggaaaaaaaaaaagccatagcattctatttctgagcattcttgttcatggacatttcattcagtttagaatcacgaaatcttaaggctggaagatcaaccagtctatcatcctgctttcacagataagaaaactgaggtcctgacatgtgaaaagattgatttttcttagatcacacagctaatagtagcagagcttaaactaaaatgcaagtatccaggctgctggttcagcattttttcaatttcttatgggagttagttctatgtgagcccagatatgtgagcacatttttgccctgacaatgatttctggcttaatttaatttctactgaatttagtgtatttacacacagacacacacagatacacacacacacacacacacacacacacacacacacgacgtaacactattttaagatacatataataggatttttatgttacagtgctataatcacctcctcagatttcttagaccaatgattgtctaaggattgtatcggcaggactaatttcatattaattgtgttccattatagctagcacgaaggctccatcccacatcttgattttaggaatagacagtgaggaatgccagttaataatcctgtaatgttcaataccttcaccttatatattatgtgtgtatgttttaattgcagtttgaatgatattgttgctgtgggacctgagcacttttatggcacaaatgatcactattttcttgacccctacttacaatcctgggagatgtatttgggtttagcgtggtcgtatgttgtctactatagtccaagtgaagttcgagtggtggcagaaggatttgattttgctaatggaatcaacatttcacccgatggcaagtatgtgaactctctgaaatgtagtggatttactcagattctcaggagatatcttggaatatattctttttttaagtaatatgatacattttaacatgttaccctagtgagataaaattaggaaaaatgtaaaatgtcttaatttttcagggggtgaaatttatcaaatcaaaaatttcattgtgaaggcatactggttttatttcctttttggaaagaaagtacattttaagagggggaactgtgatgaagatttgatatgtctgaatagctttgttttgagtaaagatgtgagtgtataatgagttgaatagtatgcaggtatagatacgtcagtagtgcaggtttatgacaagatgaggaatttgtaccaaaatgtaaatcaaggcaatacttccttcatcaagaaagtattactgtgaaaaacaagtcagtttaactaagcagtgtgcttagtgatgtatttgatttataatgttatctcatggtgagctataacaaacaatttgcaaaccaacattggtctggagactgcattttgggtagcattggtatatacatgaaatatctatatccatccacatatatgccctttctcatcttgtgtcatcatcaaggattttagagtccgtaacatatgcttagagaaagcaacaggggccaggtgtggtggttcatgcctgtaatcctagcacttcgggaggccaaggtgggtggatcacctgaggtcatagaccagcctggccatcatggcgaaaccccatctctactaaaaatacaaaaattagccaggcgtggtggcgcactcctgtaatcccagctaccagggaggccaggcaggagaattgcttgaacctgggaggtggaggttgcagtgagctgagattgcaccactgcactccagcgtgggtgacagagcaagactctgtttttttttaaaaaaaaaaaaaagaaagaaagaaagaaaagaaaaagcaagcaagcaagcaagtgagagcaacagggacagggacagcttttaattattcatatcatagaccagttttcaaaaaatagaagataattgagcagtaatttcttttttttgttggggggatgcacagtgcttagactaaccccagatctttgtggggtatgactaaagccacagggtcaggaccacactcagggcagaagaggaggaagacttctcttgatccaagccaagcagcttttcttctctcatattttctctggattaaaacaaggggctgttgtccaagggctttcctgcaactttcccgagagagaactcactagcccctgaaatgcagaacctgggccttgacactaaattgtcaaaccaaaagagaaacgctgtaatgcattttactcccagatactaaaagttagtgttagggctaagttcctcccatactgataaggacaactatgtcccctccttcagaaacaacttttcagagattaaacccctctgtcactgtaaactcactgagttctttcagaatttggtgtgtttgaggtgggttccattttccattccttaagcaaagttcttccctatgaataggtattacaacttcatcttaatctctcaagttgtgttacttctagtactttgatgtagctctttcttctttgaaggtatgtctatatagctgagttgctggctcataagattcatgtgtatgaaaagcatgctaattggactttaactccattgaaggtaagacgtattaacactgtaagtctgcagagtgataattagattctaattgatttgtgaaattaaatgtgaagaaaaaaggactgcttaattggggtgggtgagaaccaatttttcactcctttattaagagtcattagcttggttatgtgtttgttgttcatttttactcaactgcagagtggcaaacagcaaaccccacctcttacccgtgaaccttgcattctttctgctctagcaaattttaagcttggcattgtgttcccagatcctccttccatccctcccttcctgcctttcttctgatctccctctcctccaccttccttccctctcctcccttttctttctcttttttcctctatgttgatggaacagccaagaaatagcctcaaattgtactttatgcagggctgctctccacaatgtttatgaagtccacgtgctcacgatggctgctaggtgacctggctgcccctcacctctctgcctttacctccaaccattgtgtccctgccccacgtctcctgccactgtctcagaacggggcaccattccctctgctaggaggagaaaccttcctccagatctccatgaccccctccttcatctcctttcatttttactaaaacgtcacctcgatcttcaaaacctggaactgctccctcccgctacgtattttccctcgcctttcctgctttatttttccattcagcacgtattgctaacatactatatattttattcataccctttattacctgattccccaactagggtgtaagctccacaaggacaggctgtttttctatttgcttctggtacaattcacattgactagttggcacttaacaaatacttgttaaattactgcggtggggctgggcatgcttcagggattcacctgctgcccccaccaattctggtcatctgaaggttggagtcaaaatgggtgaaaacacaaaggagaggaaaaagagaccaagagcaaactagagccctaagaaagcagccctctacctccgaaaaacagcaagacgttgctttcctgccataaacgcccactgcaggaagcactgtcagcgccaattgtatgactgttcctgtcagcacatctgagagactgtcatgaggggcctcactcagagaaaattaccttaaagcaacatcaaatgtaacatcgtgtttgacattaaaaatcactggcagggctagtttggtattagctcaagtaaaatggaatttcacacgctcatactggctaagggaataaaaaacaagctgcggttttaacaacaggtatttctgtaaaacaaaagttaaagaattttatgagaatagaacccaatgtaacagacattcaccacagaatgtcaaggctctgctctcaccagggtgagactgagggcaaggccagtttcagcaacagaccaggcctgtctcccatcatgaaaaccccaaaaaggttctccatcaattgtcacattattctttcttttgaaagaccgtattaattaatatcagttaattaatgcccgagagtgccaagtaatttgataaactagctgaggctattcttaaatttgtgatgaggtgtggatgggtttatgtccaattggatattgttcagccttaaaaaggaatgaagttcttggccgggtgcggtggctcacacctgtaatcccagcactgtgggaggctgaggtgggtggatcacgaggtcaagagttcaagaccagcctggccaatatggtgaaaccccgtctctactaaaggtacaaaaaattagccagacatggtggcacatgcctataatcccagctacctgggagactgaggcaggagaatcacttgaacctgggaggcggaggttgcagtgagctgagatcacaccattgtactccagtctgggcaacaagagcaaaacgccatctaaaaaaaaaaaaaaaaaaaaaaaggaatgaagttctgatacctgctacaacatggataagccttgaaaaacatgatgctaaatgacataagccagcaacaaaaggataaatattgtacgatttactaataggaagtacctagaacaggcaaattcatggagacagaaaatagaatagaatggtgattgctaggaacttgggggagagaagaatagggagctattgtttaatgggtacaggatttctatttggggctgattaaaagttctggagagggatggtgatgatggttgcacaacattgtgaatatacttaatgccactgagttttacacttaaaaatggttaaaatgtcaaatttttatatattttaccacgatagaaataattctctcctctctccaaaaaaagattccaggacttagtcaatgaattagtcacacttcaatgaacctcaatgcgcaaaaacctggaatatttgttaacagagaatactctgtttttcccaaagagaataatttattatgttttcttgaccaaggaatgcgaatttttcgagttgcctttgtgggagtgtgagatgtgatacgcaggggtcttgcatgagttagcatggagaggaattatttatttgcaaggcaaagagttcattgctggactcagaacaggctccctgctagactgtcagcacttccatggcatgagctgtgtctgctgggtacctggtgctctccaggtgttgcgttccttgcagagctcatgtaggagctcagtaattgtgtcttgaatgaatgggaccccaggtttctgtgtccttctcccactcccactctcatctcaggtttcatctgccgtggaattcaccaattggctgtactgcttccaaagataagagcaaagccctgaaacaaacattttcctcattcaaagacattatggtatcagagtggtgtgctgacactgggatggacaggggtttagggatggagaaagcaagcatggagccgcctgtcttgcctgtctctggggtaagaaagactgatggctctgcatggtggcgagactgaggctctgccatgatgcttgagccctctaatccctccttgggcatgtgaggaacagctctcattttgagtaggaggcacagggaaacgttggttttctgcagctgtctgcttatgccctggctttttcaatgagtcatgaccagttatttacaggttctctgggcactgaggtatgctggcctctgaggtacatggtagatattatttctccaactgagaattagaggtaaacaaatagatgggatttctccccagtagaaggcagccaagatgatgaatggtggaattaagaatcattggtattatttatgatccttgtacaaagcctcgacagcaatctgtgcagtcatcaaatgcttgcaacttatatttagagaataaacagcagaagaacggggagttgaacaacatcaggaaactaaagaacaaacttttcctcaactacaaggcaaaaatgagaccctgggtgatcatttcttctctggctgcacagcctatgattagccattaaagagtccacatttcattgatattcttctgagaaagaatctacatttcctggttgttttctgaatgcttataaaaatatcattaccacatgccattttagaaggagatgttttatgatgcaccgaaaacttcttttttttttcagttttaggttcatgcacagatgttggtacaaatgattttgtctaacttttaacagtctttcttggctttctgtatcttcaccctacccaattctgccctggaaactgacaaaatactagcatagatgaaaggtcagagaaattttgtgagtcttattgaaatggggcagaattgtatccttggaagcagcagcgtggtgaccaaggacaagaggcaacttttctctgatatattttgctccatcacaattgtgttttactttatgtcttgttcttttcttgtttttaatcttttaagtcccttgactttaataccctcgtggataacatatctgtggatcctgagacaggagacctttgggttggatgccatcccaatggcatgaaaatcttcttctatgactcagagaatcctcctgcatcagaggtaagttttaaaaatgaaccagataagtgactttattcccagctactgttttttcagttgacaacataattctcaattcttggtttttttttttttttttttttttttgtgacagagtatcgctctgtcgcccaggctggagtgcagtggcgcaatcttggctcactgcaagctccgcctcccgggttcacaccattctcctgcctcagcctcccaagtagttgggaccacaggcgcatgccaccatgcctggctaattttttgtatttttttttttttttagtagagacagggtttcatcctgttggccaggatggtcttgatctgctgaccttgtgatccacccgcctcggcctcccaaagtgccgggattacaggcgtgagccaccgtgcccggccaattctcaattctatgtgcaaataaaaggggggacatggatagatgatccaaatgatcagaaatgaaggcttttggaggtgctttagtcagagtctaggcaagaaacacatggcacacttaaattggataatttgaggggagtttaatcaacggactatacacaaaatgtgtggacacagtgtagggtaatcaacagagttataatgcagtaccccagggctagcaagggagggaggaaatgaataccagaagccagaacaggtagacggtggtgggtggagagggttgcctgacaggaacttggcctctggttaagggatcctcaaagaacataatgatgccatactgctatggtttaatcattcattacctgccttcctattcaactcccccttcctgtgtggcggtatacgcacaggtccatgcaatggaccagtcctggagcacactggccagcctctctagatcttcctgcaggtggcaagataaatagctggaattattctggcttagaagatctcatcttccaccttagttacagtactcaaaggtcccattttttgtccacggtctttattcagtgaactattttgggtacaagtaagaatttgcaacctacttcaatataggtatttgcacatttactgagttttcagtatctactaaggatacttattacttacatcatatatatgatatcatcttgacatatttgtctactgctgtgtattgttaaagctcagttttaagagtccatttcacctgcccaacaggaaaaaaaaaagtgttaaagcccgtatctcttgggttaaaaaaaaaagaaacaaatgtgtatttccatttacattatggctctcttaccttttatctctaggtttgcaggacaattgtgatcatgtttaatggcaaccctttgtgccttttgctagtttttagctattagggttgctgaatggtatgtttaatagaattgaagaaatgtatgctgtggagtgttcagtgctcagaactgagcagaatgttagattctaatttgttcttgcttgttagctcatttagtaaccttgggtcatttgctatagcatggacttggtttgttgttctggagaaaatacttcatttctaatgattgatctgcaaaggagtagcaaaaagtagtaacaattccttatccaatgcactgcccctagatgagtgatttagttttattaacaagaagcttagctaagccaaggtgtaccatcaactatttccctgtaactactacatgttaatgctatggcaacttcctaagacatctgtctttggtgccagctgtttttaagaggtcattttgaacgttcccacagacccttttatatccacgcagtggccttcaccgtctgccacatttgtgatgtagatatcctttcttcaagtcattatatattatcataaaggcttaataatagaaagctagagctgaaggggaatttagagataatctagtcccacccatttcattctacagatgagaaacaaatgtgagtggaagatccaagacatgacccaggtctcccacctcagtgtagtgcttttcatactaagttgaatcacacgctattgctattttttacagtcagaatgatcagatattggctatttcatatggttggcttaacatgatactacactgttattattgatacaatcacatttctcctcaattaagtgaaggaaactgaacttacaaaatcttatacatttagagacaggaggttaagttcagaacacatgagtatggtacacagatatggttttttgagggccacattttttttcttgtctgcattttaaaaaccaggaattaagtgtaaacatccagttagttccctggcttacctgaaaatacatgaaaacggaccttaattactgcatggcaacagtttggctattgctgagtgtattagtcaaggttccctagagggacagaactaataggatatatatatatgtaaagggggtttattaagtattaacttacctgatcacaagatcccacagtaggcggtctgcaagcctgaggagcaaggagagccagtctgagtctcaaaactgaagaatttggagtctgatgtttgagggcaggaagcatcagcatgggagaaagatgtaggctgggagggtaggccagtctctcctcttcatgtttttctgcctgctttatattcgctggcagctgattagattgtgcccacccagattaagggtgggtctgccttttgcagcccactgactcaaatgttaatctcctttggcaacacgctcacagacacccaggatcaatactttgcatccttcaatccaatcaagttgactctcagtattaactatcatactgagtaacagcttcctccctccacatttactgaaatccccactgctccctaatgtcccacatccaggatgcgctgctcatttgcatttcctgcctcatccctgtaggtctagatactctccacctctgctaaagatcacttgagatcagtacccacctgttcccaaatgaagaaacaggtgtgttaagtgtaattttggaacaaaataggatttatctcattgtataagattatatggcttcttatggaggatgaccctattatgcataaggttgttttaagtgacttaacaaaaatattaacccaatgttatttcttccacaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaacagaccgatttgcacccatgccatagaaactgaggccattatttcaaccgcttgccatattccgaggacccagtgttcttagctgaacaatgaatgctgaccctaaatgtggacatcatgaagcatcaaagcactgtttaactgggagtgatatgatgtgtagggcttttttttgagaatacactatcaaatcagtcttggaatacttgaaaacctcatttaccataaaaatccttctcactaaaatggataaatcagttatgtcaattgtcagatattaaataacagtgtgtgaccccaaaagtacttaccctaaaacatgtgttgcctggaagcacatgtgtgtatcgctgccttgccatgtcttgttcagaagacacaggggagcagggttagctcacgtgtctttagaactccagtactcacccagggactccagttcacaggccagaaaacatatgcattatgaagttcccctctactccatgcacatagtaagtctgactatggcagtcagacttacttactcccattttcccttcgatatatgactttttctcagtaaatattaacctgaattattccaactccccttgtactcttgctttttcaattctcctgttgcaatgacacataggaaaatcttaaaattcttgggagtgttgtcacacctgaaaattatgagtctctatgatcttggcacaaattgtacatttgagtgtctttgacttggttaaaggaagtttgttcacttcgatgactggatacagaatgaatcccataattgacatgggcgaagctaaaagtgtccccaaagactacactgttgttgaggtggtggtagtgctggtgggtttttgtttaatatttaaacttcttgttgtggaggctgaaaagaaaaaaaataatagaaaggtaaacaaacaaataaatagaaaagatcaacaacccctttggctatctactgagacatgactaggaagaaaacatgactttatcattttgttatagaaactgatatataaaggttacacattttcatttatttgtttttctgatttgaaggtataaccttcatgatgaattacttcttcagggtgttaaggcagtgactttagaaacaaatttttttcttgtttttgttttgtttttgagaccgaatctcactctgttgcccaggctggagtgcagtggtgcgatcttggctcactgcaacttctacctccgaggttcaagagattcttgtgcctcagcctcccggatagctgggactacaggcacacaccaccatgcccggctaatttttgtatttttagtagagacggggtttcaccatgttggccagcctggtctcgaacacctgacctcaggtgatccacccgccttggccttccaaagtgctgggattacaggtgcgagccaccacacccagccagaaacgaagtttttctaaggcacttgttatgatgatagtctggcatttccgggaaaatgttttgccaataaattgagaaccaccaacagttgtttaagaatcactgacttgaagtaataaacaggaacattttgtttgggtcaaaaaaccactttattcacagcaagattggagttactcatgattccagtgacataatatcccgtgccctacatgggctcaatttttctgagatgctgctcacagcccaggatgctgggttctctgaaagactgtcttgaaattatcaatgagtgtgatgcttagggaacacccccaacatatttcattatttagaaagaaagaatttcctgagtagttaggctccctaacatcttcaataaaatgaccacattctgttctttcttgttcttgatgatagataagcaaattc

SEQ ID NO: 6 - protein, 355 aa, Artificial sequence
  Mutated PON1 protein (amino acid change L55->M)
MAKLIALTLLGMGLALFRNHQSSYQTRLNALREVQPVELPNCNLVKGIETGSEDMEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLQSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 7 - DNA, 19489 bp, Artificial sequence
  Mutated prg4 sequence; mutated base chr1186304862 (C->T); mutated codon Cgg->Tgg
taaaggaaggatctaaggaaaatatcaatacattttttaagcagatttttaaaataataaaggaagctagaaaatccagtgtcaataatctacctgtaggtaactgagagcatgttttctgtgctcagagaaaatgaaactactcaagaaatgtatatttgatataatggtataagtatataaaatgtatgccacaagctaacactaggtaaatttcaaagctacctttgctagatttaactggaaagcagaagaaataaatgataccagtatgtgaatattttaaagttgctgcttcaatatactagtcaaagtctgccagagctatttaccatctcaggacaatttgttcacttcacttagaaattagaaatgtgctctggccaatacatctaaatttattttgtggtagtcattttgcttaatggaagtgttttccagatgttgcctaagtctagtcgtctgggcccttttccaataaatgaggaaagtttgatttcataggttgtcactgttgattttgtctaacctttggactaattggttcatcccactgtatttgcactgatatataagactcccaggacgtgggataaactcatctatcctttacgggtaagtgtcaaagttaacttgcttcatagaattaaatgtgttttcattagaggtgtttgaaaaaaaatgtgtaaagatagtagttggaaatttttgaaaaggattatgtttatgcaatacacctgttggaagcctttgaatttatattgagaattaagaaaaagttggaacagtaactccatgatgcttattaaattacatttctgtgacacaggttattattttccttaagataaaattttaaccttgcactgttaagtacatgccatactttggctagagttttaagataagtctattctatattggagatttcttaagaattaaaactgtcaataacgtggctgagggtgacttgatttgttctgcataagattaagtctaatggccaaatattttctataaaattaaagctgatttaaagtcttgtttcaagaatgggatggttttattattgttacatttttaaagttactgaaatgtgtataatgcaagcctaagttagtggtgagatgaaagagctgttttctgatacttttattttattttcagcaagggtacctacggtacctgaaaacaacgatggcatggaaaacacttcccatttacctgttgttgctgctgtctgttttcgtgattcagcaagtttcatctcaaggtagcttaaccatcgaacatacttttatttaacaactattgctaatcattcagtcttgatttttataacaacggaaatatatttctaaaaatttatatttgcttgagtttaatatactgtacaaccttaaaataatatataacatgtagcctgtttgtaagtgctttggaattctcatctttttctccttttgagagttttgagcttgtataaagaataattttggtacttgttatttctatggttctctcgaaagtttaaaaagtgcagacagttcatcataaaattatgctggagctatatagtatcataaaaataactaaaaaagaacaatatttagataacatgtaatttttgcctatatgttgaacaatttgttttttaaaatcaaataaattattgataaataacttattttggcatcatttagacatagataccatattataataaactatagtgtttcaaatggcattttattagtcttgaaaatgttctctttttcttacaagaactatttatccaggtatggacatatacagatgtgactgtcattttgtgttataacattaaacttttcacagttctcccaaaacaggtcctggatgaatagttcttgttactcatttttagagactgttaaagtacattcagtgaaatcacattagtaaaagatgctagcatgccatctagggggattaatagtaacaatgccaagctttggatttttcttttcttttcttttccttttgttcccctttgataagcaaaatctgagagaataaaaatcaagattcatgacagttatgatgaaattatgtttctaaagtaaacatttccagtaaaatacgagattcttatgaaacctgaacatgagtggtaactgtctgcataggcataagttgcagaattgtttagataagaaaagacaggaaaacacatgaaagcaaatgtgaatattcaataagaatgatgactactccagtatctccagacccttcggctttctcgtaacactatgacaaggttcacaacactgggggcactttctaaactgccttttcctctgtgatacaattggttgttcactaaaacagtgttactttcattttaattgtgattaaataaatcaaattaaaattaatggggctgggtgtggtggctcacacctgtaatcccagcattctgggaggcctagggagacagatcacctgaggtctggagttcaagagcagcctgggcaacatggcaaaatcttgtctctatacaaaatacaaaaaaattagccaggtgtggtagtgtgtgcctgtaatcccagctattcggaggctgaggcaagaggatatcttgaatccaggaggtggaggttgcattaagctgagcttgcatcatggcactccagcctgggcaacagagtgagactctgtctcaaaaataaataaataaataaataaaattaatggttacaattaatagcaataaaagtaggacagacacctaatctatgaaagtaagcttttcctgtaaggatgatttcctcttttttttttttcttttgagacagggtcttgctttgtctcccagtctggagtgcagtggcacaatcttggctcactgcaacctccacctacggggttcaagcgattctcctgtctcagcctcccgagtagctgggattacaggcccctgccaccatgcccggttaatttttgtatttttagtaaagacaggttttcaccatgttggccaggctggtctcacctgacctcaggtgatccgcccgcctcagcctcccaaagtgctggaattacagatgatttcttatttcagaaatctgccaactataaaagagcaatctcttgatactgtcttgtctgcttctcttgctttctcaacctcttctcattctcttttttctttatatataatatatgtatttatatacatacactatatatacatttttgtatgcattatgcactcatgtacgcaaaaagttctgaaagttgtcctacaatttactgttttatttgcatattcagactttggcattcctggactctattcttttaagatttgttttcagtgtgtttcaacattcctttgtggatttaggacagtacacctgccaatttctattccagggatggattccattgtcacatttctgcagtcatttctcagggagggttttaaggtggtgttttccaaatgacttttaaaaaatatttgagaatgagtatgcttttgttattgtttgcttatttgttgcctggaaaacctctgttcatgacctttatagttatgcccagttattttaagggttctcacttcagcagtggttgtaagcatctgcctcccctgacattttaaaaatccagttatccccaccaattaaggaagaaaaagctcagtaaagtttatgctggtgcaaccagcgtgctttaaacttatcctcaggaatgggaggcaaagcgacaggtggaaacatctcaggcttaaaataagatatatcagagttcaaattctggatggattgtttacttaaggtgaccataaaatgtattatctaaattaggacaattataagggtaaaagagttcactacaaataattgggaggtattaattaacctatgatgccaatatactggaactattaactagtcgtgtgataatgagcaagttgtcaaaatttctgtgtttcagctttcttagctctaaaaaagggctactaaatcctggaaacattttcataagcattagtgataatgtatgactcagcacctggcatagagaagctcaataaatggcaactgctaatcatcaaaaccacagatcggtagtagctgtagctgccacctcttccttaggagtatcaccctctttaggcaaagccattactttgcctggttttcaaaaatgtgtttatcatctcagtctagtgaagaataaagtgacataattgagatcacctaagacatcagccaaatatcagctgggcctattgctgacatcattccaacaccttctcgatcaataaaattctctctcaccaagtggctttgtccccctcgttagattgctccctttctataaagtggtttggccatatttacgccagtattgtataattttagatttatcaagctgtgcagggagatgtggggaagggtattctagagatgccacctgcaactgtgattataactgtcaacactacatggagtgctgccctgatttcaagagagtctgcactgcgggtaagtcctgagagcgggtgtctcctctgtcaagcaacactgcgagtctgtgagtccccccttgcaccctcgtgcagtgctgtgagactgagcctccccttgcacccacttgcagtgctgttttcccactgtatcacttcctttgcttaagtgactttccttcacttgcataaatgttgcagcgcattgtacctaaggatggtgtcaaagcttgtgattattggggagggtaaggggagcctggaagtctgtcacttgcacaaggttattgtctagttccaagtaaatgtcttattttaaatagagaagatactgtgagtcaggagtcattaagtaaatagtaaggagtaattatttacaatcagagatgtcctcgactagtaatgaggactttcaatagtttcatcaaaagaatacatgagcatggaataatcttgtctaccattggactttctgatgttaacacgtttgtaaattacctggaaaagacactttaaagataaagtgctagtgtttaatgatgaatttcagttaaaatctgaaggtctgaattttaaattatgagtggtgtttcacttgaatatattttgggccattttattattaacacagaaacatttcctattcaatttaagaaaattgtaattaaagttatttgaaaggtgtatttgctttagaatttgaatgtgaagttgaggaatatattgtgcaatttatgtattggataggtaattatttcaaataagccttggtaagtccctgttaactctaacaaaggctttttaaattttcattttttaaatgagtagtcctccctaacatagtccacactgtaagattaggctgaaagctttcaactatacaccttccccagtttgtcataataagggcctctgcattacaaatgatttttaagcctcaaaaatgacccatttacgtggaatatatatacatatatatttatatggaatatattgctgagatctgtacattcaattactgtgaatctattacaaagcagtgtgtgaagaggagaaggatgaagagatttcatatgaaggctatctcactatctagacatttcccgatttttctttgtccatacatgtaaataactcgggcagcatcaggatgtctcttggagtctggaagggcaagaggagttgccctcagtcaccatatttcttttttgacttgggctgtctccatctgggataccatctaatttttcctggatgatgtactccaaatttcaaataaaagacttagaaatgaacttttggaaacctagtcaagtctaaggtgggaaatggctgtcaaatacgtgggcctggcttcacaatgaataatctgtaacttcttgttttgctctgggtagagctttcctgtaaaggccgctgctttgagtccttcgagagagggagggagtgtgactgcgacgcccaatgtaagaagtatgacaagtgctgtcccgattatgagagtttctgtgcagaaggtaagcatcacagtaccaaccaatgcttctcagtacagccagatctgtgcacctaccttagcatcactgataatgtctaacacgcagtccatcttaggccttgattttactaacatataagctatttcataataattttgttttgtggagatgaattaaagaagaaaagcgccttccaacagcagaacccccaggctcagccaggctccaagagtacaagatgttgccagagggggaacatagaggatgaggctgaatgtatatgtcttttttttcttcagatatggttcaatttcaggttaaatcaacagtaatcaaatcacttgatgatttcttgtctcacccttgctgacttcacgaagtcaactatcctttgagctatctgtgcttttggcctcagtgtttgagtatatatttaaggacataagcaacactttcagcttccctaaaccagtaaatttatgaattagtttaagccattaaatttataggcaaatctacctaacacgttgttaactgtcatgttccttcccatttctgcagtctcctcagcagtctctattccaatacatgtgcatggtaaagtcaataacaagggatattcccatcttaaaatcaaattctctatttataggtcttgaaggactcctctagagtataacaaaaggctccagattttacagaaaaagattttaaaaatatacttgaatactggattactttcctttaccaaatggttgttcttaaattctactttgttaaaacaagaatatttgtggaggtaaaagtgaaaataagctttgcttgcataatcaatcttatcattttatttaaacaagcctgcgcttcctgcttctttagaacagtgaagaaagatcggaaaactgctaatcagcagaattgagagatgtttcttaaccgttcaatgttttcatgggaaaacattccaaaaatgatgaagacaacacccttgtaataaaaaccttagagctcacccttcctgcagattttagcaggccccatgatgagtgacacatatttactcattaccctaattgttttggctgtgagacaagaaagctaagaatttgttttgggcagaggatgtctagggagtgaagatggttagaaagtgatgagacattgagagaacattttgataaagataatactttatgttatgaaatatttggtcagattacctaaagtttaggtagaactctgaaaaaaaaatgatgaaggcgatgctggccaactttctttacagaattcataagacagtcagcgtcccaacttaaaataataaaaccactcagaacctcaatacttcattttttaagtgtaataaccaaactatcttgagattcactacttagttgtttcagtatagatactacttacaggtgtctatatatctaaaaatcataacatttcaagaattgaaggaatcccagccatccaagtgcctctgaatgtgaaagagaaaacagagtccaagaggttaaccattttggccaagaacattagctagtacatggcagatctgtttctcaaattgagatacttgatgtttttgctgggtaggaggagaaacaagagggacagtggccagttcacggagactatagcagtagggttctggggcgatgttgcggggagcagcatgatttatcacaacccttctgtacccagagagtccccagtgctgttttcttatccttcattagtccctcttcttgttcctaaaaaaaaaaaaaaaaaagaagttccatgttttctcttctagactgagaagcggaaaagggaaggggactaaaaggttaaaaagatacacaccaaaccattaagttgtatctttgggaagggcagggcattttgaggaaaagcacaatgtgaaggtgtgaacttttcacaagaatattcatgtagaacttttgtaattaaaaaaagaacaaaaggcaaattggcatcatttaatttaaatgaatattaaaacagaaaaaaatgaatactaaaacagaaaaaaattctgaaggcaaagaccttcagaaatcatcttgaataaacatcttagtttgtagacaagaagatgaagttcaagataattatttgcctaaggtcacaatggtagaggctcgcaggcctaggactggaatttaggtcttctgattcctgagactttgctccctgtcataacttacaacaatttgagaaccagtattttatagcaatgcatccttagacttaaatggagtcattcgtgttgagctggagcctgcctgcactggctgtcaccagcatctactcttgaagctagatgcatctgtgcttttcacagttagagctgctgatgaacataaacaagatgttaactgacttgtcttacttggcctcagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaaggtaggaagatgacagatataatcaaaggagctttcttagatgaagtaacttgtaggtgactgcttatctaagcccattctcagagaacagggtaatcttaggaatcatgagcctcattacactcgaaggttttagactttgcttttaagtaaagtttaagacaaagtataaactctcagctctttctgtatttaccaaacccagcatgagtctgggttaaaacaaatcagaagggacaaatcttactataaaaaacaaaaaccaccccatgattttctttgtagaataatttgattctgtgttttggaggatatgggaaagttgagagatactagtaatactgctagtatctgtgataagcccaggtgccttgcttttaactgacagattaaaaggcagttggtcatattactaataaaagcaaaatccagatacttgtagactagtaaatagcacttcttgctgtgtttagactggtggttcttttttgttttaaatcacagttggtgtgatcaaactttctatttgatttatagaacattctgtttctgaaaatcaagagtcctcctcctcctcctcctcttcctcttcttcttcaacaatttggaaaatcaagtcttccaaaaattcagctgctaatagagaattacagaagaaactcaaaggtttgagcattgataaagatcaaagactgtatcaatgccatattaacaatgattatatttaaaagtaatgcgtgttggcagaatgaggacatcttaatatgggggggaatataactttatgaatattatcttagaaagtaccaaatgaatacttcctacaggaaattagtgccaagcggctaagaagccactctaaaagcaaaggataagaagatgctgtagcaggactgtggaaccacaagatggcaccagagatgattattgatggctttctactgtcattcttgtttacttgacatcattttgcttcttctccttcatcttccttgcattttcattttcccttccccagctttattttaacaaacatttatatactattgttctaagtgttttacaagtattaactcattaaattcttacaaacctctatggaaaaaatattatccatattttacaaatcctctttattttcctagtaacttatatattatatctggctgacttttcctctagtagagactgtgctacagaataggctattattgcctgaaaagagatttcctttgctgtttattatttcctgaagttctggttctaaatatatattctctttctccaggaaatactggatttttggataagggagttttaaagaactctaagaggttgtgatttcctccgaagcattcgctctaaataaatagccaggcatttaatcttttagtcacccagaaaatcgtgcaggaagccacccttgggctttgtagtccacatgtggggcagctgctatgggaaagccttgataaatactgtctttttgctgcccatcacaccctaagctcctccttatgtcccctgggacctgcaaggttagctgcgcagaagaaaaggagtggctaatagccctatgtatgggaacaaggctcagcagggactgtgggggtgactggggcagattgctcagaggtagctgtagaggcctctgagataattggtgcagcaagatggtagtactaggtttccaggaggaaggaagggacactgggcactgaagaataagagtacaaaaagtagggggtaaagaacattaatataaacagatgaattagtgttatttatcgtgttggtaattttgaacttgccactgacttccatgtattaattgtatagagttttatttgtgctttttcatccctcatctcatttattccctaaaacagcccaataagataggttgagtatttgccattttacaacaaattaaaggacatagtagagattagcaatttgcttagaaacacacaactagtcaatgataaagatgggatctttatgtcactattaaagaaaaaaaaatattctaaaataacaagatgtatattttttctccagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccactcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccggtattaagaatcagttattttcatttttaaagctggtaatgttagtttacatctatctgaaccagaatgccttagtttagtatcactcaactgagatatattacctgaccttgttagcttgtgaagttttacagcttccctgtaggtaagcagaggtgtaattacagtaagctttttcattctctagaaaattaagcctgggaatttagcatatgtgtgtctgtgagcagcagagatggtatatagaaagtcaaaaaaatttagttcggagtctattcttaaaagtacaatgcaaagcaaaaatctgtagtatcattttccaaactaacccaaagggagggcaatcagaactgaaagatgcaatttcacattgagttcatacatccaaaaaatgtctagtttacatgaaaatcaaggtaacctggtccattccaattcataataatttatttcatataatacttttgaagagtatcatctttaagtaaaatggaattatcaagtatataactatgcaaacaggtcaaactgtgtccttcaagatcttagaaaaggtaaacaggaaatagaaccatgtggaaagacttgaaaagaacatacagacttttctcattctgttctatatttgttttgtttttgttaatttgtttagatgagaccaatatatgcaatggtaagccagtagatggactgactactttgcgcaatgggacattagttgcattccgaggtgagctatgtacacatttatttttcttctcatcattctgttttggcatttatgagaaccaaagccgtggaagagtgctagtttgggttgttttctggaggggaatctgattgataagcacacctcacaggagaatccgagagtaataacagaaatattggattaagaaaattagacaaagtaacaaaacaaaaccaagaccctgaactgatactactgaggttttttcctactgttaagatttaaaaagtagttcattttttttggggggggggggtagttaaaatttaaaaaataaaataaaattttaatttcataccaaatgaaatgacaccagcatgcatattcactgctttatttgaaaataacatctcaattgtgatgtgttcaagtgttacactctctgtcggagagtaacagaagaaaagttatttcatgttaattctctgctgtactctgctagtccagtcttgaaacatatttagattaaatctgaaagcaattccttttcgtacagtagaggctaaagagatgttagtcattaaattataattccttagttctaagagataaagaaattgagatctaaagagatagtgactgtaagtcacaaagccaaaaggcttttttgagaccggatctccctctgtcacccaggctggagtagagtggtgcaattacggctcaatgcaggctcgatctcctgggcccaagtgatcctcccatctcagccacctgagtagctgggactacaggtgtgcaccaccatgcctggctaatttttgtatttttttttgtatttttttttttttgtagagacagggttttgtcatggtgtccacgctggtctcaaactcctgggcccaagccatctacccgccttaacctcccaaagtgctggattacaggtgtgagccactgcacctggcccaaccaataggctctttctaaagaaactagaaaactaaagtatctcaaaggttatgctaaatttcggacttgggaaatacctgctctcagaaatgttcaaatactaccttttgtatctgactaaacttccagtcatacaatgcataagaaaactacattttttttcattttggtttcttttgtgataggtttagcattcagcttgtaggctgatgtctttccttaaattttaggtcattatttctggatgctaagtccattcagtccaccatctccagctcgcagaattactgaagtttggggtattccttcccccattgatactgtttttactaggtgcaactgtgaaggaaaaactttcttctttaaggtaacacaaatatctttgtgagagaaatttaataataatttgtaactgcattatttattacaacatatattgccttaacctccaatatgtgtccttttaaatactctgtcatcaaaatttaatcataattcaacagtttgataacagtaatggtcaaaattcaaccgtttaaccaagttaacacgttctctctatacttaaatctagaaaggagttccaaatcttttttcccaaagctgataacataattttctcttttaaattatcaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtatgtgttacataattgacaagattacaaaacttttaaagaattacactcagcattttcatttattattgactttactgtcaaaagatatctttaatggctgaaatcaaatattttcagtgaaaaaaatcaatattgagggtagtattcttattgccctcaatttttattttcatttcttcacaggcaagtcacaattcatttgagttttcaggtcactgatagaatgtcatttactttcgcttcacaaatgtgcatgtcatccttgcacaagggccatgctaatctctgtatcattccagttttagtatatgtgctgccaaactgagcacttgtcactttcaattgaaattaaatatataactatgtaatttgctgcatctattcattcaacaagtatttcagtttaataattatttttataatacccttgactaatagccattattaatatcaatatattatatgaaaaatgagaacaaaactgtttcctatacattgtaaaagttgttttccaccttttctgagggtattaaaattaattttcattttccatgtgatattctaatacataacaggtggcagcattcagcagtatatttataaacaggaacctgtacagaagtgccctggaagaaggcctgctctaaattatccagtgtatggagaaacgacacaggttaggagacgtcgctttgaacgtgctataggaccttctcaaacacacaccatcagaattcaatattcacctgccagactggcttatcaagacaaaggtaacatttttattgtgttaaaaaaatccttaaacataagtagatgtaatacagtttcttatacagtaaggcttatgaaatatggatactgatcaaacagccctaataatttaagcttactgatgaaaaactcatccacttgccttagtgaataaccaaagaccaatactttctttttccttgtgggtaagtaaagcagtttgttcaggtttgttagttataaccaatactatttatcaagtacttcttaccaagaacattatataccagtctataaccctcaatagttctacatcagagggctaaaactgagattaaaactcagatgtctggctctttccaagatagtacattgccttttaatctggtatcttttattaaacatgccacttacaggtgtccttcataatgaagttaaagtgagtatactgtggagaggacttccaaatgtggttacctcagctatatcactgcccaacatcagaaaacctgacggctatgattactatgccttttctaaaggtaaggtattaactaacagtttcccaaggaggtgatatcatttgtgaaaacatgaagataaagtaaaaatgctggctgcaatgatgactgaatgtgagaagttacactacggtacttattctaattgctgtggaaaagagttaagacttttgctttaatttcaattaaaatgatggcactgcaattcaattctgctctaaccttcatgagattacgataaaacatccagttactagagtaaacttgctactgacaatagtattttacttctatcatttgtggcatttaacagataccttgtttggcacaaggttataatgatttgcatcactgcagagtctgtaagatcagaaagaagttcctgagaaactaattttattaattaaattatattactcaggatgcttttctttgaaatgcccagaaatctaagttattctgcctgtaatttcataatatgaatgacattggcatgtattttttaaaaggaataaccccaagtaaattatttttcaaacttggttactctttaatgtttacttcataaaggagagctgaacctgattttacaaaaagatggtgaaatgtcaatcttttgaaacatcaaaaaagctgaaaagtctaggcaattgtttttctttttgttataaagttcaaattaattagtaaaaacatctctattaaaatgataaacattgtctttgagcataatagtcaacataagttattttttagtttgggcattgttttctttttaactaaaaaaatgttttctcttccatttagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaggattagaaaacttgttccatttattactattccacattctatcatgtaatccagttatgtcagaaattcacctctctgtctgttaaactgacgaccacggacaccttgtctcaatgttgtctgaagtcttactcgtctgaaaggctttggttgactactactagtatctaagaaaaacattaagataaaagaaaagcaagtgaaaaaatgagaaagcatttataatgatacaaactaattactaaatgaatgaatgaataagatgtagacttgttcatttgatttcaattctttttttcaaacaatgaggaacatttaacaaaccgaaaaagggtaaatggttaaacaacaaaaaaagcagcactttgggaggctaaggtgggaggatcacttgagctcaggagttcgagaccagcctgggcaacacagcgagaccctgtttctacaaaattataaaacaattagctgggcatggtggaacatgcctacagtcccagttactcaggaggctgaggctagaggatcacttgagcccgggtgaggttgaggctgcagtgagccatgatcaagctaacagtactccagggagaccggtgagatcctgccttaa

SEQ ID NO: 8 - DNA, 13649 bp, Artificial sequence
  Mutated epx gene sequence; mutated base chr1758193733 (G->C); mutated codon caG->caC;
gcaccttggagggagtgagtagggctaggggataaggagaggggccaaatcattcatcacagatcattcattcactcaacaaacacctgctaagcacatactaaatgctgggctttgagctgggtatggggggtgtgtataacagtaaggcagattcagtttctgccctgttgcaggaggagaggcacccttccaatcatctggcccaactctcattctagggatgtgaaaactgaggcctggagagggaaagtggcttgcccaaggccatgcagccaggcggtggcgactgaggtgcaccgctcctgtcagccaacaaatatccattgagcgacacctgtgtcccaggtgctgctctgggccctgggagaagtgcatcagtgggcttggtagtagagggtagggatggagtgaagggtaggcaggaagaatgtccccaggctggtaggaggtggggtggggggtttcagtctcaaaactcccatgaaaaccagagagaagtttcagaactccacccaagaggctgggtttctagggcccagagctgccctcccccaccctagaatgggctataaaagtcccttcccagctacgtccagagaagagctggaggaagtgagaggtcggctgggggtcctcaaagtgagaggggagcagaggatcctcccgtgcaggctgtggatgtcactcacttcccagctggtgaagcctcgctgcagagatgcatctgctcccagccctggcaggggtcctggccacactcgtcctcgcccagccctgtgagggcactgacccaggtaatagtcccctagacaggcaaggaggagggaggggaaatggaaggggaagcacttgggtcttggagggggtcttgtggcttgctgaaccctgagtccccatctctttgaacagcctcccctggggcagtggagacctcggtcctgcgagactgcatagcagaggccaagttgctggtggatgctgcctacaattggacccagaagaggtggacttgggtctgggggctgcatgggcctgggaggatcagtgaggggcatgggccccagccaagttcatctcactcatcagccacctctggaccccatgaacatttcctgttggtagagcctcccttccatccatccttctgtccgcttgccctgtcctgactgtgccccaggactgggtctctgctgggtgggtctgcaccctctctccagccctcactcctcctctcctgggcagcatcaagcagcggcttcgcagcggttcagccagccccatggacctcctgtcctacttcaaacaaccggtagcagccaccaggacagttgttcgggccgcagattatatgcatgtggctttggggctgcttgaagagaagttacaaccccagcggtccggacccttcaatgtcactggtactctgatccccactgagcccgctgggcctaccctggcctggagtagaaggaatccaggagagggaggcagggtgcacaggtttggggtgctgggaggagagagggtaaagggatgggaggtacagagcaggccagctcaggtctgcccatttgccttcccacagatgtgctaacagaaccacacctgcggctgctgtcccaggccagtggctgtgctctccgggaccaggccgagcgctgcagcgacaagtaccgcaccatcactggacggtgcaacaacaagtgcgtgcggggcggcaggaggggctgcccctgcctgggggacctctcccttcctgcacccaccctctccctccatgctgagccatctccaggccctgccccctgctaacctatcccacccatggctgcaggaggagacccttgctaggggcctccaaccaggctctggctcgctggctgcccgccgagtatgaggatgggctgtcgctccccttcggctggacccccagcaggaggcgcaatggcttccttctccctcttgtgagttggggctgagggtttgggaggttgcttgatctcttaaatgcggggagtaaaacacagcccagagtcacgcaggcagggcttgaacccaggcccttccactgaccagtgcagtgacttgaggcaaataacacagctttctgaggcttagtttcctcatctgtaaaatggccctaaaacctacctcgtagagcctgtgtggatattgaagtccttggtataaatagctatagaggacatgatctattcatgctaaccattacggtgttgtacggttttttctgagaaaattagtgtcaaagtcttgctaaatataaacagctctataccttaatctggctttatctattgatctatttcacatttatcaactcatcagttttctattgatatatcgatagagcagtctatcatctacccatctatcaacatctattaattaattttcttctatccacccttccatctgctgaccccgtactccctacgtgtattatccatcacctggcaaccagcccatgcacctgtgaatccgtgctatcatctggttagctatcagtgtacaaattgatctatctatttgatctcccttcccctctgggagagctggtgaggtctgagccagtcaacctagcccctctccttctctttaccaccggaatcctcaggagcccagccagaaaccatccttctaggaatgagagcaggaggtggctacgcctccagggacaaaaggggcatggagggcagaagaggagaggctgtcaattccagcaggggagctgctgctccctgagtcctgggttggctctaataccttgtggggtcagggagcccatgtcccgtgctgatgttatttccccaccaggtccgggctgtctccaaccagattgtgcgcttccccaatgagagactgacctccgaccgtggccgagccctcatgttcatgcagtggggccagttcattgaccatgacctggacttctccccggagtccccggccagagtggccttcactgcaggcgttgactgtgagaggacctgcgcccagctgcccccctgctttcccatcaaggtacctaccctcagccaatctcccatgcccttgtgtggcctcccccaaaggcaaggtgctgggggtggggatctggaagactggagcaccatccttaaggagctgcctgtggagctagggtatgagacagagacacaagaaacacagctgagcagagaccccgcgccgtgtgtgtttgagaggtgggggtagggcaatctgccaggaggctcaggtcaggcttcatggagtgggaagcccttgacacatgccttgacccatgggtctgaatccaacaggggaagcctctgggcccctgcttttggcaacctaagggcctcttagctcttgcccttctctccttcccagagtagtccaagggtcctgcggctcttgccagcttcttgggcttgggctgtaagtagggtcctaaccttcaggctagaagccaaggacagtgtggggcactaggggaggaaagatagaatccagggagcagagtctctgctaggaacatagggttgacacacgtgcacacacacactctctcacacacacacacgtgcacacacacgctccctctcacacacacatgtacacacacagtcacccttaataggtggggccacattcatgagggatgacatgtgagggcctctggatggcctctccctgttgcaaccttatctattcctcaatccctggcttaggggcacctcctctggggagccttctcagatcttcctattacacttttaacatatgtcctcccctcccctctcctctctctccttccttccttcttccttcctctttctttctttcttctttctttcctttccttccttccttcccttccttccttcatttccttccttcctttctttctttctttttctttctttttctttcttccgtcctttctttctttatctttccttctttctttcttctttattattattattattgttttgagacagaatctcactctgttgtccagactggagtacagaggggcaatctcagctcactgcaacctctgcctcctgggttcaagcagttctcatgcctcagcctcccgagtagctgggattacaggcatgtgccaacaagcctggctaatttttgtatttttagtagagattgggtttcaccatgttgacaaggctgatctcaaacttctggcctcatgtaatccacctgcctcggcctcccaaagtgctaggattataggcgtgagccaccacacctggcccacatatggcttttctatcaagcacagcagatgccacttcaacttagtttcttaactctatgtgtgtgtgtctttttccactgaactgccagcttcctgcaacctttcctgtgtttttttaaccttcataatctccaaagcagcaagcaagaaaggggcttagaaaaggtctgtcgaattgaacagaattaaagaagctcaggcctaagagtcaagagaactaagttctaatcccacctttcctgctaactggttgtcacttcccctctctgaggctgttttccttgctgtaaaatgaggatcgagattgtttcaaaagcctcattcattcctaaccttttgagactctatgaaacaaaatgttaaggacactcagactgacggggtgagcagcatgagcctgggtgagtcaaggagggcttcctggaggaagggagttttaagcaaggttttgaggaggtggaagatgacggagaggagtgagtctgctattgagggggccccatgtcactgtctcctcttccatctcagatcccacccaatgacccccgcatcaagaaccagcgtgactgcatccctttcttccgctcggcaccctcatgcccccaaaacaagaacagagtccgcaaccagatcaacgcgctcacctcctttgtggacgccagcatggtgtatggcagtgaggtctccctctcgctgcggctccgcaaccggaccaactacctggggctgctggccatcaaccagcgctttcaagacaacggccgggccctgctgcccttcgacaacctgcacgatgacccctgtctcctcaccaaccgctcggcgcgcatcccctgcttcctggcaggtcagacagggaggaaggtggtgtcttcccaggaaacagccatccctggggtcccaactgggaagcaatggtgggatgtggtgaaggtacatggtttgggacctcagtattaggcacaccataagcatggatctgtgcacagccatcatagaatcagaacgttgagtccctttgcaagctccctgtgtgtggccagagataactgcttaaccgtctccagtgacaaggctctcaccgcctctatggcaccccagctcagccacaggcagctccgactgttagaaagtctccttattttgatcacatattcacctattccccttaatcatgcttctggaacctcagaaaagacactgtccctcttacacgcaggaatacctcaaagtagctgttaactccatctccattttttttctcttctctgggataaatttccgtagttcctccctgtccccagctgttgctagacagttaataccctcttctggaagcataatttgaactttcctcagggtcttttgtcccatgtacatggttacaataataatggtagtcatttctcattaagatatttatcttattattattattattttaagatggagtctcactctgtcgcccaggctggagtgcagtggcgtgatctcagctcactgccacctctgcctcctgggttcaagtgattcccctgcctcagcctcccgagtagctggaattacaggtgcctgccaccacgtttggctaatttttgtatatttagtggagatagagtttcaccatgttggccaggctggtctcgaactcctgacctcaggtgattcacctgccttggcctcccaaagtgttgggattacaggtgtgagccaccacgcccaaccttatcaagatatttaaatgtgccatctctaaaccttgcaacagccctgaaaggagggaattattattgttctcattttacgataagaaactgagactcagggaaagtgaatcatgcgcccaagagcacagagttagtttcaaatgacagagtccacttgagaacacagtcagtgtagtatcaaggtctctgcccagtccactgtattccactgttttcctcacagagagtcggccccaacccctcttttgaaggattgtacatttgtgtgtcctggaaagagcagcttgtgccttcgggagaggtcaactgaactaagaccttgtgcaaaagggtttagttaaaaccaagcaaatgatggaattaactttctttccctgcctgtagtaggtctgttcgtaaggacagtctctgcccaaccctccctctcttcctctttccctctggttcacgggggagagcagtattataccaagcccaaagataggaagaggtacaccagcccttggcagagaacacgcaggcagatgcagctcatggacaggtgggctctcagtcccaaagtcaaggtgcccttgcccatagctggtccaggctatggatctggttgtcttgtagagtagaatccttaggcaaagatgcccctgcctgagaccagcagctgccctgtgctttcccacctcacccaagcctgctaggaacccccggtccttccgctcctggcctgtggtgttagggaggagggtggcgatgaggagcctcagcacagctgaagagatggaggtccagtgagggccaggagtttggcccaccccgtctctcccatccccagccctgggtctaccctggtagaaagacatttctctgggaaaggctgcagtaaatctgagcttggggttttcaaggtgacacccgatcaacggaaacccccaaactggcagccatgcacaccctctttatgcgagagcacaaccggctggccaccgagctgagacgcctgaatccccggtggaatggagacaaactgtacaatgaggctcggaagatcatgggggccatggtccaggtaaggagctctgcatcccagcatcccccagatgacaagcttggcatgagaagcagtccttaacacatccttgcggatgtgcctaaaaccagctgggtctgggcaactggcggagcacctggacctgtcctctggccccgattctgcctctaactcccgtgtgaccctgtctgtgtcactcaccctctctgggctttgtatctccacccaccaatagtaaattaatgttgtcacatttgacgtgatgacaataaagaatatgtctgagccaccctttgaaaaggcaagggtatgggtgagtagcctctggggaatgttcctcctgtcttcccttccagatcatcacctaccgagactttctgcccctggttctgggcaaggcccgggccaggagaaccctggggcactacagggggtactgctccaatgtggacccacgggtggccaatgtcttcaccctggccttccgctttggccacacaatgctccagcccttcatgttccgcttggacagtcagtaccgggcctccgcacccaactcgcatgtcccacttagctctgccttctttgccagctggcggatcgtgtatgaaggtgaccaggttttccagggggcaaatgggggtgagggtggggagcatgccctcccctaggtgggccaagcttactgccaggaagccaggctgctgcagaggccactgctaatatctccccaggacagtggaaacaaggcaggtgccagcaagaccctcagtcacgggcctcccatcctgtgtggaatgagaggattttttaaaggggtggagactaatgtcagattgatggggagctcaccttccattccttaagaagtacctcccagctccagctgcttcatgtctctccagaactctgtttcctgacaaacgttactaacatacccgactggcttgtccagctctgggctagcttggcatcatgtgataacccaagtagcttcccagaggctggtccaatctgtgctgctcacattccctgccaccagggggcatcgaccccatcctccggggcctcatggccacccctgccaagctgaaccgtcaggatgccatgttagtggatgagctccgggaccggctgtttcggcaagtgaggaggattgggctggacctggcagctctcaacatgcaacgaagccgggaccacggccttccaggtgagggggctgtccacctcttctcccagctttgctcgggccaggctgctcaaggggttctgggaagaccctggtacctcctttctgactgggactgtctacaggatgtgcaggagtgcagaggcatgcaaggccaaggtcgatgtcccaaagcactcctggaacaccgcttgctgtcctgccatggctcccccatccactgtaggggctccagctttaggttcaccacaggaccagcacttaggctcaggagctgaaaaagggaggctcgaactttccaagtagaaaaaaagcaataggcttaaaaggcagggaagaactgctccattcccaacacgctggcaagttccaaggaggaaggctaaaattccagagctacagaaacgctccttgctctaagtggagaaaggcaaagagccttagaggcagggaataggtggaataaaactaaggctgcagtggctgtttggtgagggcacaaatgactttatcccaggaaagaagaaaatataggcactttgttagtcactttcatgtatattaatttattttgttctcccaatcaccttgagaagcaaatgattacagccattttatagatgagaaactatgttcagagacagctagtaatttgcccaagatcacataaataagaagtagtaaaatctgaatttaaatttacaatttctcctttcttccccctgttcaaccccagtaggggaaggttttggccactgccttgctcctctctccttcccacctcctacctccctgtctagaaagaacctgaaggcccatctgcccatgtatgaagcagtggtggcacctcctactgcccaccccacttcacacagcgcctggtagagcacagcaggcactccggaaacctgagcttctttcctttcttaaacttgggcgctcctggcagctaaggagctgtgggcaccccctgccggagagctgagatcgctagccacccaagtttctattaggttttgaagggaaggcaagggttaaggaaagacacaaagagagagggcggctgaacagccaatgcaggtatttatttccagcataaaacctacagaggtgggggaccagcttattgccagaacctgccgccgcctacagctggggtactgggtatgggtatgggtatgggcaggaggggtctgggtcgtatggcttgctacccagcaggatattgataagatgtacctatgatcaggtccttttttcagtgggatgtggtaagatgttccttggacctttgcccagcaggatatgatagggatgttccttcagttgggcctttgcccattagggtatgttatgtttttcatggcccgacctcctgtggaacgtttcactttgaccaaggtctgggaaatggtggggggcttagaaaatggtgcagtttggactaacattcttgccttctactttggtataaaaggaagagctggagagctgagatcgctagccacccaagtttccacattgcgagcttctgctccccgcaatacctgtgccctccagaaatattatcctgacactagctcttgcaatctcctctaatcccttcccaagtccaacatcaaactgggaactggacaacctggtttgagctccacttaacagtaaagtgaccctgaagcctatggttgctcaactttagtaaacataccagatatagttagcatgccataaaagcacctcaaaaaggtgctgtgagggtcctgagatattcacccaatagtgctcagtaatggtaaaatactgtgtggttttcatgggttccgctgggggatggtgtgtgtgagctgtagttcaatggatgacgttgggctggtgtcccttcgggacatggcaaagctatctcctaccttgattgtcacatcttatgatgtggcttggccctgcgtgggatcctgatggctgacaaattggtatggcttgtctttggaagtttctgaaaactttttaaaaacttctttattgagatataccataaaattcacccatatgaaatagacaattcaatgattttagtgtatttagagttatgcacccatcaccatgatctaatatcacaccattttcactccccaaagaaaccttgtaagaacagtcaatccccatccccaccccagccccaatcacctctaatatactttgtgtctctatattgacctatgcaggacctctcatatcaacagaatcatagtgtgccgtccttttctgagtagatgaattttatcattgctctccatttgtttgcttgttcattcatttattcatttgctcactcattcattctacaaacctgatggagagtcttttatattctgggtactgtcttacttgtgtctaatgaatgcagtcgagcccagtaatatcactaagtgtcggatttgtggttatgagttttgggttaaaacagctttttaggttaggacacactcctcagtctgatgcacactgagcacagttgtccactcacattttcaatcagaggacagaaacctagtctttggaagctctagggactcttctgccagcaggatcttctggttctgcccaatattgactggccacagcttccccccagaggactggtggagaaaaacagaagctaatgggagatcagcaagactgaagctgcttctccccgttcccctgcagggtacaatgcttggaggcgcttctgtgggctctcccagccccggaatttggcacagcttagccgggtgctgaaaaaccaggacttggcaaggaagttcctgaatttgtatggaacacctgacaacattgacatctggattggggccatcgctgagcctcttttgccgggggctcgagtggggcctcttctggcttgtctgttcgagaaccagttcagaagagcccgagacggagacaggtaagtgaccctatcataaaagacatcagcaccagaggcagagcagaaaaacactagcatttcaagactaaacattgaagaacactgctctttttagtatcatttcttccaagttcacaggatctgaaatcaggaggctcctctctgaaaagctgggtcaaaggaagagagacactgacccaggcaaggcccatattgcctgagctggagtcatcttaaacccagaggttgcctggcagcctcactattctgggttggtggatcccagaaaacatgggcagaaagggctaaatctggtttccctccaatacatttgtgatttaagacctgacggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccggctacttgggaggctgaggcaggagaatggcgtgaacccgggaggcggagcttgcagtgagccgagatcctgccactgcactccagcctgggcgacagagcgagactccgtctcaaaaaaaaaaaaaaaaaaaaaaaaaaaaaaaaaaaaaaagacctgtctccttatttggagttggtattgcccgagttcaaatccagccctggccacttaaattactgtgcacttaatactgggcaatttatttggccactgaatttgtttcatttctgagattccaatcttgcaggaattttgtgagaattgaatggaataatatatgtaaagtacctggcacacaacaggtgctcattataaggtaattcctccccagccttcacccacatctctcgactgcctggtaggttctggtggcagaaacgaggtgttttcaccaaaagacagcgcaaggccctgagcagaatttccttgtctcgaattatatgtgacaataccggtatcaccacggtttcaagggacatcttcagagccaacatctaccctcggggctttgtgaactgcagccgtatccccaggttgaacctatcagcctggcgagggacatgaggcttctgcaggtaaggggaggccacctccagcaccctgggctgggttaagccctcacatccttccctggatggatggctgagtcctcttaggtctctaagcagagaaaacagaacttgtcactaggtactctttccaagtggcttcccaatgtgctagtttctgggctgacagtcaattccaggccctaggactttggggggaaattaggagcatccaactatcctcagcttggtcactaatacctgaaaacagctccatttctaaacatccagtaaattcctgacttatccagttctgttacttccacaggagtctatcccaagtctccaacttttggagacaaggggaaggggaggaccatgaggctgccttgtctccctggagcaagtgcaggctgctgacgcttctgctggctacagctcagagctgggttccccagccaggagtgaaggctgggggctcctatcagcaatggaccttcccgccttgggagcctcttaggtattaggctatgaatcagcgccacgtgcaaaggcttgggagccaagccatgtggtcttgcaccccaggcaagaaaagtcagctggagggtttacagcactttctactgtttcccagccctccctcccctccctcaccatgactaagagaccactcggtcctagcctccagacaccccacaatactcctctgagcctgaggccaggcagcatgctctgcttctaccaataaagcactgctaagggcaggggctgctgtttctgtgttgggctagggaagacgtggcctcagttccatgctttgctccagatgtttaagagaagtgctgttaacacgcaagctggcacaaccatctaagcctctcctgggggctgaattaggagagcccaaagaaacactgtggagtagggaaaggcagaaaaggggaagggcagacatggtaggggccccgacattaagaacaccctagccataccagcaacctgagcagacaaacagccaacagaacatcagtcaaaataaaaggggtttatgtaacaaaaaacaaaaaaacaaacaaacaaaaaaacacagtaaaagataccacccagctagcagaaaggactcagcactgccttcagccttcacttactcagaaataactcatatctcaacctcagcaagccccaagcagtagaatgaggcttccctggaaagaggctgagactcacaggctggggatttaagaccctctcaccgtccaccttccttccttctttggtcttggaagatgaaaggctccatttttaaagggtggagaacagcagatcccctgctactgtgagacaagccagaaaagtgagtcaag

SEQ ID NO: 9 - DNA, 28057 bp, Artificial sequence; identical to SEQ ID NO: 5
  Mutated PON1 gene squence; mutated base chr7 95316772 (A->T); Mutated codon Atg->Ttg;